SEQUENCE LISTING (6 sequences)

SEQ ID NO: 1 - DNA, 23 bp, Artificial Sequence
  EBNA1 forward
atcagggccaagacatagagatg

SEQ ID NO: 2 - DNA, 20 bp, Artificial Sequence
  EBNA1 reverse
gccaatgcaacttggacgtt

SEQ ID NO: 3 - DNA, 20 bp, Artificial Sequence
  FBX15 forward
gccaggaggtcttcgctgta

SEQ ID NO: 4 - DNA, 20 bp, Artificial Sequence
  FBX15 reverse
aatgcacggctagggtcaaa

SEQ ID NO: 5 - DNA, 300 bp, Artificial Sequence
  EF1alpha promoter p1
gagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagacc

SEQ ID NO: 6 - DNA, 300 bp, Artificial Sequence
  EF1alpha promoter p2
gagaaggtggcgcggggtaaattgggaaagtgatgtcgtgtattggtttcgtttttttttcgagggtgggggagaatcgtatataagtgtagtagtcgtcgtgaacgttttttttcgtaacgggtttgtcgttagaatatagttgaagtttcgaggggttcgtattttttttttacgcgttcgtcgttttatttgaggtcgttatttacgtcggttgagtcgcgttttgtcgtttttcgtttgtggtgttttttgaattgcgttcgtcgtttaggtaagtttaaagtttaggtcgagatc

(19) United States
(12) Patent Application Publication
(10) Pub. No.: US 2022/0362300 A1
(43) Pub. Date: Nov. 17, 2022

(54) GENERATION OF CHIMERIC ANTIGEN RECEPTOR MODIFIED T CELLS FROM STEM CELLS AND THERAPEUTIC USES THEREOF

(71) Applicant: City of Hope, Duarte, CA (US)

(72) Inventors: Stephen J. Forman, Duarte, CA (US); Christine E. Brown, Duarte, CA (US); Zhiqiang Wang, Duarte, CA (US)

(21) Appl. No.: 17/774,088

(22) PCT Filed: Nov. 5, 2020
(86) PCT No.: PCT/US2020/059216

§ 371 Date: May 3, 2022

Related U.S. Application Data
(60) Provisional application No. 62/931,125, filed on Nov. 5, 2019.

(51) Int. Cl. A61K 35/17; C12N 5/0783; C12N 15/62; C12N 15/90; C12N 15/11; C12N 9/22; C07K 14/725; C07K 14/705; A61P 35/00

(52) U.S. Cl. CPC A61K 35/17; C12N 5/0636; C12N 15/625; C12N 15/907; C12N 15/11; C12N 9/22; C07K 14/7051; C07K 14/70596; A61P 35/00; C12N 2506/45; C12N 2310/20; C12N 2800/80; C12N 2501/16; C12N 2501/155; C12N 2501/165; C12N 2501/115; C12N 2501/2302; C12N 2501/2307; C12N 2501/2315; A61K 38/00

(57) ABSTRACT

Methods for preparing T cells or NK cells expressing a chimeric antigen receptor (CAR) is described. The methods entail: isolating a population of T cells, generating induced pluripotent stem cells (iPSCs) from the T cells, introducing a nucleic acid molecule encoding a CAR into the iPSCs to create CAR iPSCs; and differentiating the CAR iPSCs into CAR T cells or CAR NK cells.

### CLAIM OF PRIORITY

This application claims the benefit of U.S. Provisional Application Ser. No. 62/931,125, filed on Nov. 5, 2019. The entire contents of the foregoing are incorporated herein by reference.

### TECHNICAL FIELD

This disclosure concerns the generation and use of chimeric antigen receptor modified T cells from stem cells or progenitor cells.

### BACKGROUND

Chimeric Antigen Receptor (CAR) T cell therapy is a cancer treatment that genetically alters T cells to redirect and harness their cancer killing potential. Currently FDA approved CAR T cell products are autologous-based, requiring individualized blood apheresis and manufacture. Deriving patient-specific CAR T cell products is expensive, laborious, and time consuming, with numerous logistical and regulatory challenges.

Generating CAR T cells from induced pluripotent stem cells (iPSC) holds encouraging prospect for generating ‘off-the-shelf’ CAR T cell products and overcoming these challenges. iPSCs can proliferate almost infinitely while keeping their pluripotency and lineage differentiation potential. However, the complexity of T cell development and disturbance of T cell differentiation by CAR expression creates a challenge for successful iPSC-derived CAR T cell generation.

### SUMMARY

Described herein, inter alia, are methods for making and using phenotypically defined, functional, and/or expandable T cells or NK cells expressing a chimeric antigen receptor (CAR) from pluripotent stem cells embryonic stem cells (ESCs) or induced pluripotent stem cells (iPSCs). The CAR T cells and CAR NK cells described herein target a specific predetermined antigen expressed on the cell surface of a target cell, possess enhanced functional potential, enhance survival and treatment of cancers and/or targeted diseases, and/or possess cytotoxic potential and anti-tumor activity. The CAR T cells and CAR NK cells described herein may be used as “off-the-shelf” cells for administration to multiple recipients, which crosses immunogenic barriers and alleviates at least symptom of Graft versus Host disease (GVHD).

In some embodiments, naïve and memory T (T_{n/mem}) cells-derived iPSCs are start material for generating iPSC-derived CAR T cells. In some embodiments, peripheral blood mononuclear cells (PBMCs), naïve T (Tₙ) cells, memory T (Tₘₑₘ) cells, naïve and memory T cells (T_{n/mem}), or a combination thereof-derived iPSCs are start material for generating iPSC-derived CAR T cells. Without being bound by theory, T cells already have the TCR gene rearranged during development, and the T-derived iPSCs maintain the rearranged TCR loci, which is important for T cell development during in vitro differentiation. In some embodiments and without being bound by theory, T_{n/mem} is a subpopulation of young T cells with premium fitness comparing to terminal differentiated effector T cells. The generated T_{n/mem}-derived iPSCs may also have unique properties because of less epigenetic footprints.

In some embodiments, described herein in a method for preparing a composition of CAR T cells, the method comprising:
(a) isolating a population of peripheral blood mononuclear cells (PBMCs), naïve T (Tn) cells, memory T (Tmem) cells, naïve and memory T cells (Tn/mem), or a combination thereof;
(b) generating induced pluripotent stem cells (iPSCs) from the PBMCs, Tn cells, Tmem cells, or Tn/mem cells, or combination thereof;
(c) contacting the iPSCs with a vector encoding the CAR, thereby creating CAR iPSCs; and
(d) differentiating the CAR iPSCs into CAR T cells.

In some embodiments, described herein in a method for preparing a composition of CAR NK cells, the method comprising:
(a) isolating a population of peripheral blood mononuclear cells (PBMCs), naïve T (Tn) cells, memory T (Tmem) cells, naïve and memory T cells (Tn/mem), or a combination thereof;
(b) generating induced pluripotent stem cells (iPSCs) from the PBMCs, Tn cells, Tmem cells, or Tn/mem cells, or combination thereof;
(c) contacting the iPSCs with a vector encoding the CAR, thereby creating CAR iPSCs; and
(d) differentiating the CAR iPSCs into CAR NK cells.

In some embodiments, the PBMCs, Tₙ cells, Tₘₑₘ cells, or T_{n/mem} cells, or combination thereof are human or are isolated from human blood. In some embodiments, the PBMCs, Tₙ cells, Tₘₑₘ cells, or T_{n/mem} cells, or combination thereof are CD14⁻, CD25⁻, and CD26L⁺.

In some embodiments, the PBMCs, Tₙ cells, Tₘₑₘ cells, or T_{n/mem} cells, or combination thereof are reprogrammed to generate iPSCs. In some embodiments, the iPSCs are generated by contacting the PBMCs, Tₙ cells, Tₘₑₘ cells, or T_{n/mem} cells, or combination thereof with one or more of OCT3/4, OCT3, OCT4, SOX2, KLF4, L-MYC, C-MYC, LIN28, or short hairpin RNA targeting TP53 (shRNA-TP53). In some embodiments, transduced cells are cultured in X-Vivo15 medium supplemented with 50 U/mL IL-2, 0.5 ng/ml IL-15 and CD3/CD28 Dynabeads (bead:cell ratio of 1:1). In some embodiments, one, two, or three days after the transfection, equal volume of PSC medium containing bFGF and 10 μM Y27632 is added. In some embodiments, three, four, five, six, or seven days, the medium is then completely changed to PSC medium. In some embodiments, the iPSC cells are cultured at least 1, 2, 3, 4, 5, 6, 7, 8, 9, 10, 11, 12, 13, 14, 15, 16, 17, 18, 19, 20, 21, 22, 23, 24 26, 27, 28, 29, or 30 days. In some embodiments, individual colonies are picked to further cultivation and evaluation.

In some embodiments, CAR iPSCs are generated contacting the iPSCs with a nucleic acid or vector encoding a CAR. In some embodiments, transduced CAR iPSCs are cultured for at least 2 passages before single cell sorting and iPSC colonization. In some embodiments, colonized CAR IPSCs are expanded and banked for differentiation.

In some embodiments, the IPSCs or CAR iPSCs are genetically modified. In some embodiments, one or more genes are knocked out, down regulated, or upregulated.

In some embodiments, the one or more genes comprise one or more of TRAC, TRBC, B2M, CIITA, or combinations thereof. In some embodiments, TRAC, TRBC, B2M, CIITA are knocked out. In some embodiments, TRAC, TRBC, B2M, CIITA are down regulated. In some embodiments, genetic modification is achieved by methods described herein and those known in the art. In some embodiments, genetic modification methods comprise gene editing, homologous recombination, nonhomologous recombination, RNA-mediated genetic modification, DNA-mediated genetic modification, zinc finger nucleases, meganucleases, TALEN, or CRISPR/CAS9.

In some embodiments, CAR iPSCs are differentiated into embryonic mesodermal progenitor (EMP) cells and further differentiated into CAR T cells. In some embodiments, the EMP cells are CD56+ and CD326−.

In some embodiments, the CAR-expressing iPSCs are differentiated into embryonic mesodermal progenitor (EMP) cells and further differentiated into CAR NK cells. In some embodiments, the EMP cells are CD56+ and CD326−.

In some embodiments, the CAR iPSCs are differentiated into CD34+ hematopoietic stem and progenitor cells (HSPCs) and further differentiated into CAR T cells.

In some embodiments, the CAR iPSCs are differentiated into CD34+ HSPCs and further differentiated into CAR NK cells. In some embodiments, the CAR iPSCs are differentiated into CAR T cells using a nanofiber matrix-based culture system.

In some embodiments, the CAR iPSCs are differentiated into CAR NK cells using a nanofiber matrix-based culture system.

In some embodiments, the CAR is specific for a tumor, cell surface marker, and/or toxin. In some embodiments, the CAR targets any one or more of carbonic anhydrase IX (CAIX), carcinoembryonic antigen (CEA), CDS, CD6, CD7, CD10, CD19, CD20, CD22, CD30, CD33, CD34, CD38, CD41, CD44, CD49f, CD56, CD74, CD123, CD133, CD138, CS1, chlorotoxin receptor, an antigen of a cytomegalovirus (CMV) infected cell (e.g., a cell surface antigen), epithelial glycoprotein (EGP 2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptors, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), light chain kinase insert domain receptor (KDR), Lewis A (CA19.9), Lewis Y (LeY), LI cell adhesion molecule (LICAM), melanoma antigen family A, 1 (MAGE-AI), Mucin 16 (Muc-16), Mucin 1 (Muc-1), Mesothelin (MSLN), NKG2D ligands, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor R2 (VEGF-R2), Wilms tumor protein (WT-1), or combinations thereof.

In some embodiments, the CAR is bispecific.

In some embodiments, the chimeric antigen receptor comprises: at least one targeting domain, a spacer, a transmembrane domain, a co-stimulatory domain, and a CD3ζ signaling domain. In some embodiments, the CAR is a 1928z.

In some embodiments, described herein is a composition comprising the iPSC-derived CAR T cells or CAR NK cells. In some embodiments, a composition comprising iPSC-derived CAR T cells or CAR NK cells has enhanced therapeutic properties. In some embodiments, the iPSC-derived CAR T cells or CAR NK cells demonstrate enhanced functional activity including potent cytokine production, cytotoxicity and cytostatic inhibition of tumor growth, e.g. as activity that reduces the amount of tumor load.

In some embodiments, a composition comprising the CAR T cells comprise one or more of helper T cells, cytotoxic T cells, memory T cells, naïve T cells, regulatory T cells, natural killer T cells, or combinations thereof. In some embodiments, a composition comprising the CAR T cells comprise CD3⁺, CD5⁺, CD7⁺, and TCRαβ⁺. In some embodiments, a composition comprising the CAR T cells comprise CD8+ CAR T cells are CD8αβ T cells, which have strong cytotoxicity against tumor cells in an antigen specific manner and can potently secret cytokines such as IFNγ. In some embodiments, CAR T cells have predominant homogenous TCR phenotype. In some embodiments, a composition comprising the CAR T cells comprise CD3+CD5+CD7+TCRαβ+CD8αβ+, CD3+CD5+CD7+TCRαβ+CD4+, CD62L+CD45RA+ stem memory T cells, CD62L-CD45RA-CD45RO+effector memory T cells and CD62L-CD45RA+ effector T cells, and combinations thereof.

In some embodiments, described herein is a method of increasing survival of a subject having cancer comprising administering a composition comprising a CAR T cell or CAR NK cell described herein.

In some embodiments, described herein is a method of treating a cancer in a patient comprising administering a composition comprising a CAR T cell or CAR NK cell described herein.

In some embodiments, described herein is a method of reducing or ameliorating a symptom associated with a cancer in a patient comprising administering a composition comprising a CAR T cell or CAR NK cell described herein.

In some embodiments, a composition comprising a CAR T cell or CAR NK cell described herein is administered locally or systemically. In some embodiments, a composition comprising a CAR T cell or CAR NK cell described herein is administered by single or repeat dosing. In some embodiments, a composition comprising a CAR T cell or CAR NK cell described herein is administered to a patient having a cancer, a pathogen infection, an autoimmune disorder, or an allogeneic transplant.

In some embodiments, the cancer is selected from the group consisting of blood cancer, B cell leukemia, multiple myeloma, lymphoblastic leukemia (ALL), chronic lymphocytic leukemia, non-Hodgkin's lymphoma, ovarian cancer, prostate cancer, pancreatic cancer, lung cancer, breast cancer, and sarcoma, acute myeloid leukemia (AML).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1E show surface marker profiles of T_{n/mem} iPSC-derived CAR T cells.

FIGS. 2A-2B show TCR Repertoire of T_{n/mem} iPSC-derived CAR T/T cells and conventional PBMC-derived CAR T or T cells. Cells were stained with IOTest Beta Mark TCR VP Repertoire Kit together with APC-antiCD3 antibody. CD3+ cells were gated to analyze the TCR VP Repertoire.

FIGS. 3A-3D show T_{n/mem} iPSC-derived 1928z CAR T cells with potent antigen-specific cytotoxicity against CD19+ target cells in vitro.

FIG. 4 shows iPSC 1928 CAR T with potent antigen-specific cytokine production.

FIGS. 5A-5D show iPSC 1928 CAR T with antigen-specific degranulation and activation.

FIGS. 6A-6D show iPSC CAR T cells with potent anti-tumor activity in vivo.

FIGS. 7A-7D show surface marker profiles of T_{n/mem} iPSC HSPC-derived CAR NK cells and cord blood CD34+ HSPC cell derived NK cells.

FIGS. 8A-8B show cytotoxicity of iPSC-derived CAR NK cells against different tumor lines.

FIG. 9 shows degranulation activity of iPSC CAR NK cells against tumor cells.

FIG. 10 shows phenotype of iPSC CAR T cells generated by nanofiber matrix-based co-culture system.

FIGS. 11A-11B show surface marker profiles of colonized iPSC lines expressing CARs.

FIGS. 12A-12B show surface marker profiles of iPSC-derived CAR T cells. A. iPSC CAR T phenotype at week 7 without REM expansion; B: phenotype after REM expansion.

FIGS. 13A-13I show generation of iPSC-derived CD19-CAR T cells. (13A) Schematic of events (top), cell type (middle) and media conditions (bottom) during PSC-ATO culture. Reference online STAR Methods. (13B, 13C) Seven-week organoid cultures of iPSC CD19-CAR T cells with GFP+ DLL4⁺ MS5 feeder cells were fixed by 2% paraformaldehyde and stained with CD3 (red) and DAPI (blue) in situ. White bars indicate scales of 500 μm (13B) and 100 μm (13C). (13D) Number of differentiated T cells derived from 1 million mock-transduced or CD19-CAR expressing iMP. Data of three separate experiments is depicted, with mean±S.E.M as bars. (13E) Expansion of iPSC-derived Mock T and CD19-CAR T cells. 1×10⁶ T cells were expanded as indicated in (13A). Data of three separate experiments is depicted, with mean±S.E.M as bars. (13F) Representative flow cytometric analysis of the indicated markers on conventional (Conv.) vs. iPSC-derived mock-transduced (Mock) and CD19-CAR expressing T cells. Percentages of cells expressing each marker are indicated in the relevant quadrants, which were drawn based on isotype control staining. (13G) Percentages of cells staining with the indicated markers in three separate experiments, with mean±S.D. as bars. (13H) Comparison of transgene expression levels on conventional (Conv) vs. iPSC-derived Mock T and CD19-CAR T cells. Top, representative histograms of EGFRt staining as a marker for CAR expression, with mean fluorescence intensity (MFI) indicated. Bottom, transgene MFI data of three separate experiments is depicted, with mean±S.D. as bars. *, P=0.0011 using Student's t-test. (13I) TCR Vβ repertoire of conventional vs. iPSC-derived Mock T and CD19-CAR T cells.

FIGS. 14A-14F show gene and signaling signature of iPSC CD19-CAR T cells. (14A) Principle components analysis (PCA) and (14B) hierarchical clustering of global transcriptional profiles of two samples of iPSC, conventional (Conv.) mock-transduced (Mock) or CD19-CAR T cells, iPSC-derived Mock T or CD19-CAR T cells, or conventional PBMC-derived NK cells. (14C) Vocano plots of iPSC Mock T vs Conv. Mock T cells (left), or of iPSC CD19-CAR T vs. Conv. CD19-CAR T cells (right). Top five upregulated genes in conventional cells are highlighted with green dots, while those in iPSC-derived cells are highlighted by red dots. (14D) Heat map of z score value of T lymphoid related genes, cytotoxicity mediators, inhibitory markers and NK receptor genes. (14E) Bisulfite converted genomic DNA was used as a template for PCR analysis using methylation-specific primers (MSP) and unmethylation-specific primers (USP) within the EF1α promoter. (14F) EF1α promoter methylation determination by bisulfite sequencing. Region 114-360 bp of EF1α promoter was PCR amplified from bisulfite converted genomic DNA, sub-cloned, and 6 clones for each group were sequenced. Number of methylated CG sites for each clone, out of the 23 CG sites in this 245 bp region, are indicated at the right of each row.

FIGS. 15A-15K show the functional profile of iPSC CD19-CAR T cells. (15A), Brightfield images after 4 hour co-culture of iPSC-derived mock-transduced (Mock) or CD19-CAR T cells with CD19+ 3T3 cells at an effector-to-target (E:T) ratio of 4:1. White bars indicate scale of 100 μm. (15B-15E) Cytotoxic activity of iPSC CD19-CAR T cells against CD19+ or CD19-negative/knockout (CD19KO) NALM6 (15B, 15C, 15E), or Raji (15D) target cells when co-cultured at the indicated E:T ratios for 4 h (15B, 15E) or 48 h (15C, 15D). Lytic activity was compared to that of iPSC-derived mock transduced T cells (MOCK, 15B) or conventional CD19-CAR T cells (Conv., 15C). Mean±S.D. values of duplicate cultures are depicted. *, P<0.001 by two way ANOVA test in (15E). (15F) Cytotoxic activity of iPSC-derived (iPSC) or conventional (Conv.) CD19-CAR T cells against patient derived ALL cells when co-cultured at the indicated E:T ratios for 4 h. (15G) Degranulation (i.e., surface CD107, left) and intracellular IFN-g levels (right) in iPSC-derived mock-transduced (Mock) or CD19-CAR T cells was measured by flow cytometry after co-culture with the indicated stimulator cells (X-axis labels) at an E:T ratio of 1:1 for 5 hours in the presence of the Golgi Stop protein transport inhibitor. *, P<0.01 by Students t-test. (15H) Flow cytometric analysis of activation markers were compared between iPSC-derived Mock T and CD19-CAR T cells that were unstimulated (None), or stimulated with CD19+ or CD19-negative/knockout (CD19KO) NALM6 at an E:T ratio of 1:1 for 24 hours. Percentages of CD3+ cells expressing CD25 or CD137/4-1BB are indicated in each contour plot, with gates drawn based on isotype control staining. (15I) Cytokine production by iPSC-derived or conventional (Conv.) Mock T or CD19-CAR T cells was measured by Bio-Plex analysis of supernatants harvested 24 hours after co-culture with CD19+ or CD19-negative/knockout (CD19KO) NALM6 cells at an E:T ratio of 1:1. *, P<0.001 by Student's t-test. (15J) T cell exhaustion marker profile of iPSC-derived or conventional (Conv.) CD19-CAR T cells after being re-challenged by CD19+ NALM6 cells every 2 days for a total of 3 stimulations at an E:T ratio of 1:2. Cells were stained with anti-PD-1, anti-TIM-3, anti-LAG-3 and percentage of CD3+ cells staining for no (0+), one (1+), two (2+) or all three (3+) markers were determined by flow cytometry. (15K) Western Blot analysis of ERK, phosphorylated ERK, PLCγ, PLCγ phorphorylated at Y782, PLCγ phosphorylated at Ser1248, endogenous CD3ζ, phosphorylated endogenous CD3ζ, CD3ζ within the CAR, phosphorylated CD3ζ within the CAR, or GAPDH as a loading control in the indicated T cells cultured for 60 minutes alone, or with NALM6 tumors that are either CD19+ or CD19-negative (CD19KO). Tumor cells cultured alone were also examined as controls.

FIGS. 16A-16F show that iPSC CD19-CAR T cells demonstrate potent anti-tumor activity in vivo. (16A) Schema of animal studies using intraperitoneal (i.p.) tumor model. On day −4, NSG mice were inoculated i.p. with 2.5×10⁵ ffluc+ NALM6 cells. Mice were then either left untreated, or treated with 6×10⁶ iPSC-derived mock-transduced (Mock) or CD19-CAR T cells i.p. on days 0 and 3; in one group receiving iPSC CD19-CAR T cells, 2×10⁷ irradiated NS0-hIL15 cells were also administered 3 times a week for 3 weeks. Tumor burden was determined by weekly bioluminescent imaging. (16B), Geometric mean±95% CI of i.p. tumor ffLuc Flux over time. Using two-way ANOVA test: *, P=0.0008, ** P<0.0001. (16C), Kaplan-Meier survival analysis of i.p. xenografted mice. Using Mantel-Cox test: *, P=0.0034 comparing the iPSC CD19-CAR T treated group to the non-treated group; **, P=0.0016 comparing the iPSC CD19-CAR T+NS0-hIL15 treated group to the iPSC CD19-CAR T treated group. (16D) Schema of animal studies using intravenous (i.v.) tumor model. On day −4, NSG mice were inoculated i.v with 2.5×10⁵ ffluc+ NALM6 cells. Mice were then either left untreated, or treated with 5×10⁶ iPSC-derived CD19-CAR T cells i.v. on days 0, 3 and 6; where indicated, 2×10⁷ irradiated NS0-hIL15 cells were administered 3 times a week for 3 weeks. Other control groups included mice that received 2×10⁶ donor-matched Tn/mem-derived Mock T at day 0. Tumor burden was determined by weekly bioluminescent imaging. (16E), Geometric mean±95% CI of i.v. tumor ffLuc Flux over time. Using two-way ANOVA test: *, P=0.0019, **, P=0.0002, ***, P<0.0001. (16F) Kaplan-Meier survival analysis of i.v. xenografted mice. Using Mantel-Cox test: *, P=0.0035 comparing either iPSC CD19-CAR T treated group to the non-treated group.

FIGS. 17A-17E show derivation of iPSC from Tn/mem. (17A) Morphology of representative Tn/mem derived iPSCs. Bright field (left) and alkaline phosphatase stained (right) images of iPSCs on MEF feeders (top) or in feeder-free conditions (bottom). Black bars indicate scales of 200 μm. (17B) Flow cytometric pluripotency marker profile of a representative clonal iPSC line reprogrammed from Tn/mem. Percentages of cells expressing each marker are indicated; SSC, side scatter. (17C) Examination of integrated plasmid DNA in iPSC clones by PCR. Primers specific for EBNA1, as plasmid integration marker, and FBX15, as a loading control were used. Lane 1, H₂O negative control; Lane 2, positive control: iPSCs electroporated with 10 ng EBNA1 containing episomal vector; Lane 3-11, clonal iPSC lines. (17D) Flow cytometric profile of representative mock-transduced (top) and CD19-CAR+ (bottom) iPSC lines that had been re-colonized, expanded and banked. As clinical vector incorporated the EGFRt selection marker, which is co-expressed with the CD19-CAR, it was used to detect transgene-expressing lines. (17E) Representative results from a teratoma formation assay using Tn/mem derived CAR+ iPSCs. Yellow arrows: ectodermal derived tissue (neuronal rosette); white arrows: mesodermal derived tissue (muscle, cartilage and connective tissue); blue arrows: endodermal derived tissue (gland like tissue). White bars indicate scales of 10 mm (left panel) and 200 μm (H&E panels).

FIGS. 18A-18F show extended phenotype of iPSC CD19-CAR T cells. (18A) H&E staining of organoids from iPSC CD19-CAR T cells at week 7 of iMO-ATO culture. (18B-18D), Representative flow cytometric profiles of resulting iPSC CD19-CAR T cells before (18B) and after REM expansion (18C, 18D). (18B, 18C) Percentages of cells expressing each marker are indicated in the relevant quadrants, which were drawn based on isotype control staining. (18D) Single parameter histogram comparison of the indicated T cell lines. Grey histograms, T cells stained with isotype control antibodies. (18E) TCR Vβ repertoire of starting PBMC (CD3-gated) and Tn/mem cell populations (top), and long-term cultured (35 days) conventional (Conv.) mock-transduced (Mock) or CD19-CAR+ T cells (bottom). (18F) PCR fragment analysis for TCRβ genomic rearrangement in TCRB clonality kit controls (left), as well as conventional (Conv.) vs. iPSC-derived Mock T cells (middle) and CD19-CAR T cells (right). Brackets indicate relevant size ranges of 170-210 bp and 285-325 bp PCR fragment analysis for TCR genomic rearrangement.

FIGS. 19A-19C show gene and signaling signature of iPSC CD19-CAR T cells. (19A) Heat map of z score value of positive selection related genes including TCR rearrangement and MHC genes (left panel) and reported T cell exhaustion related genes (Crawford et al., 2014; Gattinoni et al., 2011; Long et al., 2015) (right panel). (19B) Bubble plot showing top up- or down-regulated signaling pathway derived from GSEA comparison of iPSC CD19-CAR T cells vs. conventional (Conv.) CD19-CAR T cells. (19C) Sequence of EF1α promoter where 115-359 bp region to be amplified with forward (F1) and reverse (R1) primers contains 23 CpG islands (indicated by ‘+’ signs). The methylation of these CpG islands is depicted in FIG. 14F.

FIGS. 20A-20B show mouse study information. Bioluminescent images of NSG mice from i.p. (20A) or i.v. (20B) models depicted in FIG. 16. Red ‘X’es in indicate groups/mice were euthanized due to disease burden.

### DETAILED DESCRIPTION

The invention is further described in the following examples, which do not limit the scope of the invention described in the claims.

### Methods for Examples 1-3

DNA Constructs

CD19-targeted CAR (1928zCAR) and IL13Ra2-targeted CAR constructs were the same as currently used in clinical studies targeting B cell leukemia/lymphoma (clinicaltrials.gov #NCT01815749) (Wang, X., et al., Phase 1 studies of central memory-derived CD19 CAR T-cell therapy following autologous HSCT in patients with B-cell NHL. Blood, 2016. 127(24): p. 2980-90) and recurrent/refractory GBM (clinicaltrials.gov #NCT02208362) (Brown, C. E., et al., Regression of Glioblastoma after Chimeric Antigen Receptor T-Cell Therapy. N Engl J Med, 2016. 375(26): p. 2561-9).

The 1928zCAR comprised a CD19 scfv domain, a CD28z costimulatory domain, IgG4 spacer with two point mutations (L235E and N297Q) within the CH2 region, the cytoplasmic a truncated human epidermal growth factor receptor (huEGFRt) as a safety switch (Jonnalagadda, M., et al., Chimeric antigen receptors with mutated IgG4 Fc spacer avoid fc receptor binding and improve T cell persistence and antitumor efficacy. Mol Ther, 2015. 23(4): p. 757-68; Urak, R., et al., Ex vivo Akt inhibition promotes the generation of potent CD19CAR T cells for adoptive immunotherapy. J Immunother Cancer, 2017. 5: p. 26; Wang, X., et al., A transgene-encoded cell surface polypeptide for selection, in vivo tracking, and ablation of engineered cells. Blood, 2011. 118(5): p. 1255-63).

The IL13Rα2 CAR construct comprised of a human GM-CSF receptor a chain leader peptide, a human IL-13 with an E13Y mutation, an IgG4 spacer with 2 point mutations (L235E and N297Q), a CD4 transmembrane domain, a human 4-1BB costimulatory domain, and the cytoplasmic domain of human CD3ζ. In some embodiments, a truncated CD19 was also introduced in the construct to allow for potential enrichment and tracking of transduced cells (Brown, C. E., et al., Regression of Glioblastoma after Chimeric Antigen Receptor T-Cell Therapy. N Engl J Med, 2016. 375(26): p. 2561-9; Brown, C. E., et al., Optimization of IL13Ralpha2-Targeted Chimeric Antigen Receptor T Cells for Improved Anti-tumor Efficacy against Glioblastoma. Mol Ther, 2018. 26(1): p. 31-44).

Tn/mem Isolation

Blood products were obtained from healthy donors under protocols approved by the COH IRB, and naïve and memory T (T_{n/mem}) cells were isolated following the similar procedures described in previous studies (e.g. Wang, X., et al., Phenotypic and functional attributes of lentivirus-modified CD19-specific human CD8+ central memory T cells manufactured at clinical scale. J Immunother, 2012. 35(9): p. 689-701). In brief, PBMCs were isolated by density gradient centrifugation over Ficoll-Paque (GE Healthcare) and then underwent sequential rounds of CliniMACS/AutoMACS (Miltenyi Biotec) depletion to remove CD14- and CD25-expressing cells, followed by a CD62L-positive selection for T_{n/mem}.

Generatation iPS Cells from PBMC or Tn/mem

Reprogramming PBMC or Tn/mem cells into iPS cells was conducted with procedures similar to published protocol (e.g. Okita, K., et al., An efficient nonviral method to generate integration-free human-induced pluripotent stem cells from cord blood and peripheral blood cells. Stem Cells, 2013. 31(3): p. 458-66). In brief, 1-3 million PBMC or Naïve and memory T (T_{n/mem}) were electroporated with 3 μg plasmids mixture by using Nucleofector 4D electroporation device (Lonza). The plasmid mixture was composed of episomal plasmids encoding OCT3/4, SOX2, KLF4, L-MYC, LIN28, and shRNA for TP53. The transduced cells were cultured in X-Vivo15 medium supplemented with 50 U/mL IL-2, 0.5 ng/ml IL-15 and CD3/CD28 Dynabeads (bead:cell ratio of 1:1). Two days after the transfection, equal volume of PSC medium containing bFGF and 10 μM Y27632 was added. The medium was then completely changed to PSC medium 4 days after transfection. iPSC colonies were shown at day 20-30 and individual colonies were picked to further cultivation and evaluation.

Generation of CAR Positive iPS Cells and Colonized iPS Cell Line

iPS cells were routinely cultured in cGMP grade mTeSR1 medium (StemCell Technologies) in matrigel coated plates. Before lentivirus transduction, the iPS cells were dissociated with accutase treatment and single iPS cells were seeded in density of 10⁵ per well in 12 well plate in mTeSR1 medium with supplement of 1X cloneR and 10 μM Y27632 (StemCell Technologies). After overnight culture, clinical grade lentivirus encoding CAR was added into the culture with 10 μg/mL protamine sulfate to transduce the iPS cells (multiplicity of infection [MOI]=1). The transduced cells were cultured for at least 2 passages before do single cell sorting and iPS colonization. Colonized CAR positive cells were expanded and banked for differentiation use.

iPSC Gene Editing

Certain genes, including TRAC, TRBC, B2M, CIITA, were knocked out by CRISPR-Cas9 gene editing technology using ribonucleoprotein (RNP) complex delivery. In brief, 180 pmol of chemical modified guide RNA with scaffold and gene specific target sequence was mixed with 60 pmol Truecut Cas9 protein (Thermofisher Scientific) in 50 ul P3 primary nucleofector solution (Lonza) and incubated for 10 min at room temperature to form RNP complex. iPS cells were dissociated with Accutase treatment. 1×10⁵ single iPS cells were washed in PBC with 10 μM Y27632 and spun down by centrifuging at 300 g for 3 min. The supernatant was carefully removed and the cells were resuspended in 50 uL P3 primary nucleofector solution and followed by combining with the RNP complex solution. The combined cell suspension was transferred to the cuvette and electroporation was performed with Nucloefector 4D instrument (Lonza). After electroporation, 500 ul mTeSR1+1XcloneR was added into the cuvette and incubated for 15 min in incubator before transferring into Matrigel coated 6 well plate. The cells were cultured in mTeSR1 medium with supplement of 1X clonR and 10 μM Y27632 for 2 days. The medium was changed to mTeSR1+cloneR. After passaging for two times, single cell sorting were performed and colonized iPSC cells were cryopreserved. Genomic DNA were extracted and targeted PCR and sequencing were performed to screen for edited colonies.

Generation and Isolation of Human Embryonic Mesodermal Progenitors (hEMPs)

Mesoderm commitment was induced as previously described (e.g. Montel-Hagen, A., et al., Organoid-Induced Differentiation of Conventional T Cells from Human Pluripotent Stem Cells. Cell Stem Cell, 2019. 24(3): p. 376-389 e8; Chin, C. J., et al., Genetic Tagging During Human Mesoderm Differentiation Reveals Tripotent Lateral Plate Mesodermal Progenitors. Stem Cells, 2016. 34(5): p. 1239-50; Evseenko, D., et al., Mapping the first stages of mesoderm commitment during differentiation of human embryonic stem cells. Proc Natl Acad Sci USA, 2010. 107(31): p. 13742-7). Briefly, human pluripotent stem cells (hPSC) were harvested as a single cell suspension after Accutase (StemCell Technologies) treatment, washed, and counted. Cells were resuspended directly in X-VIVO 15 medium supplemented with rhActivin A (10 ng/ml) (R&D Systems, Cat. 338-AC-010), rhBMP4 (10 ng/ml) (R&D Systems, Cat. 314-BP-010), rhVEGF (10 ng/ml) (R&D Systems, Cat. 298-VS-005), rhFGF (10 ng/ml) (R&D Systems, Cat. 233-FB-025), and ROCK inhibitor Y-27632 dihydrochloride (10 μM) (Tocris, Cat. 1254). Cells were plated on Matrigel coated 6-well plates at 3×10⁶ cells per well in 3 ml. Medium was then changed daily with X-VIVO 15 supplemented with rhBMP4 (10 ng/ml), rhVEGF (10 ng/ml, and rhFGF (10 ng/ml). At day 3.5, cells were washed 3 times with PBS and incubated with Accutase (1 mL per well, for 10 min. at 37° C.). Cells were harvested using a cell scraper, washed in PBS, and stained with antibodies for flow cytometry. CD326-CD56+ hEMP were isolated by FACS on a FACSARIA cell sorter (BD Biosciences, San Jose, Calif.) or by CD56 enrichment kit (StemCell Technologies).

Differentiation of CAR+/− T_{n/mem} iPSC into CAR T/T cells by EMO ATO Culture System (Protocol IA)

T_{n/mem} iPS cells with or without CAR expression were induced to differentiate into EMP (CD56+CD326−) cells and then further to T cells according to published protocol (e.g. Montel-Hagen, A., et al., Organoid-Induced Differentiation of Conventional T Cells from Human Pluripotent Stem Cells. Cell Stem Cell, 2019. 24(3): p. 376-389 e8). Firstly, embryonic mesodermal organoids (EMO) culture were set up by aggregating EMP cells and MS5-hDLL4 feeder cells. MS5-hDLL4 cells were harvested by trypsinization and resuspended in hematopoietic induction medium composed of EGM2 (Lonza) supplemented with 10 μM ROCK inhibitor Y-27632 (StemCell Technologies) and 10 uM TGF-βRI inhibitor SB-431542 (SB blocker). At day −14, 5×10⁵ MS5-hDLL4 cells were combined with 0.5-1×10⁴ purified hEMP per PSC-ATO in 1.5 mL Eppendorf tubes and centrifuged at 300 g for 5 min at 4° C. in a swinging bucket centrifuge. Multiple (up to 12) EMOs were prepared per tube. Supernatants were carefully removed and the cell pellet was resuspended by brief vortexing and resuspended in hematopoietic induction medium at a volume of 6 μl per EMO. 6 μl of cells were plated as EMOs on a 0.4 μm Millicell transwell insert (EMD Millipore) and placed in 6-well plates containing 1 mL hematopoietic induction medium per well. Medium was changed completely every 2-3 days for 1 week, with medium composed of EGM2 with SB-431542 10 μM. This medium was changed every 2-3 days. At day −7, medium was change to EGM2+SB blocker (10 μM) with the hematopoietic cytokines rhTPO 5 ng/ml (Peprotech 300-18), rhFLT3L 5 ng/ml (Peprotech, Cat. 300-19), and rhSCF 50 ng/ml (Peprotech, Cat. 300-07). At day 0, PSC-ATOs were initiated simply by changing the medium to “RB27” supplemented with 10 ng/ml rhSCF, 5 ng/ml rhFLT3L, and 5 ng/ml rhIL-7. Medium was changed completely every 3-4 days. After differentiation culture for 5-7 weeks, PSC-ATO CAR T cells or T cells were harvested by adding MACS buffer (PBS/0.5% bovine serum album/2 mM EDTA) to each well and briefly dis-aggregating the ATO by pipetting with a 1 mL “P1000” pipet, followed by passage through a 50 μm nylon strainer.

Differentiate CAR+/− T_{n/mem} iPSC into CAR NK/NK Cells by EMO-ATO Culture System (Protocol 1B)

T_{n/mem} iPS cells with or without CAR expression were induced to differentiate into EMP (CD56+CD326−) cells and then further to CAR NK or NK cells by a similar protocol with protocol 1A (above) with modification of feeder cells and cytokine combination in the step of ATO culture. Briefly, the feeder cells in 1B would use MS5_DL1 instead of MS5_DL4. The 10 ng/mL IL15 was added along with other cytokines (10 ng/ml rhSCF, 5 ng/ml rhFLT3L, and 5 ng/ml rhIL-7) from day 0. T_{n/mem} iPSC CAR NK/NK cells were harvested on day 28-50.

Generation and Isolation of CD34+ HSPC from iPSC Cells

iPS cells were differentiated into CD34+ hematopoietic stem and progenitor cells (HSPC) by using STEMdiff Hematopoietic Kit (Stemcell Technologies). Briefly, iPS cells were harvested and seeded as small aggregates in mTeSR1 medium. After one day culture, the medium was changed to differentiation medium A to induce the cells toward a mesoderm-like state. On day 2, half-medium was changed with fresh medium A. On day 3, medium was changed to B and half-medium changed was performed on day 5, 7, and 10 to promote further differentiation into hematopoietic cells. The hematopoietic progenitor cells were harvested from the culture supernatant on day 10-12. CD34 positive enrichment kit (Stemcell technologies) was used to enrich the CD34+ HSPC cells.

Differentiation of CAR+/− T_{n/mem} iPSC Derived HSPC into CAR T/T Cells by ATO Culture System (Protocol 2A)

T_{n/mem} iPSC derived HSPC cells were differentiated into CAR T/T cells using published ATO culture system {Montel-Hagen, 2019 #9; Seet, 2017 #19}. In brief, MS5-hDLL4 (or MS5-DLL1, as noted) cells were harvested by trypsin treatment and resuspended in serum free ATO culture medium (‘RB27’), which was composed of RPMI 1640, 4% B27 supplement (thermofisher scientific), 30 uM L-ascorbic acid 2-phosphate sesquimagnesium salt hydrate (Sigma-Aldrich), 1% penicillin-streptomycin, 1% Glutamax, 5 ng/ml rhFLT3L and 5 ng/ml rhIL-7. MS-hDLL4 were combined with enriched CD34+HSPC in 1.5 ml microcentrifuge tubes and centrifuged at 300 g for 5 min at 4° C. in a swinging-bucket centrifuge. Supernatants were carefully removed and the cell pellet was resuspended in ATO culture medium at a volume of 6 μl per ATO. 6 μl of cells slurry were plated as ATOs on a 0.4 mm Millicell transwell insert (EMD Millipore) and placed in 6-well plates containing 1 mL RB27 per well. Medium was changed completely every 3-4 days. After several weeks' culture, the generated CAR T/T cells were harvested by adding MACS buffer (PBS/0.5% bovine serum album/2 mM EDTA) to each well and briefly dis-aggregating the ATO by pipetting with a 1 mL pipet, followed by passage through a 50 μm nylon strainer.

Differentiation of CAR+/− T_{n/mem} iPSC Derived HSPC into CAR NK/NK Cells by ATO Culture System (Protocol 2B)

T_{n/mem} iPS cells with or without CAR expression were induced to differentiate into CAR NK or NK cells by a similar protocol with protocol 2A (above) with modification. Briefly, the feeder cells in 2B were MS5_DL1 instead of MS5_DL4. 10 ng/ml rhSCF and 10 ng/ml IL15 was supplied in RB27 along with other cytokines (5 ng/ml rhFLT3L, and 5 ng/ml rhIL-7). T_{n/mem} iPSC CAR NK/NK cells were harvested on day 28-50.

Differentiation of CAR+/− iPSC into CAR T/T Cells by Nanofiber Matrix Based Culture System (Protocol 3A)

In this protocol, when hMEP cells were ready at day −14, MS5-hDLL4 cells were harvested by trypsinization and cell suspension was irradiated for 80 Gy. For each well of 6 well plate, one Millicell transwell insert (EMD Millipore, pore size 0.4 μm˜3 μm) containing a polymeric nanofiber insert (Nanofiber solutions, ECM matrix) was placed and 2 mL medium was added into the well outside the insert, 2.5×10⁵ EMP cells and 5×10⁶ MS5-DLL4 cells were mixed, resuspended in 250 ul medium, and seeded directly on the nanofiber matrix insert. The hematopoietic induction medium comprised EGM2 (Lonza) supplemented with 10 μM ROCK inhibitor Y-27632 (StemCell Technologies) and 10 uM TGF-βRI inhibitor SB-431542 (SB blocker). Medium was changed completely every 2-3 days for 1 week, with medium composed of EGM2 with SB-431542 10 μM. At day −7, medium was change to EGM2+SB blocker (10 μM) with the hematopoietic cytokines rhTPO 5 ng/ml (Peprotech 300-18), rhFLT3L 5 ng/ml (Peprotech, Cat. 300-19), and rhSCF 50 ng/ml (Peprotech, Cat. 300-07). At day 0, the medium was changed to ‘RB27’ supplemented with 10 ng/ml rhSCF, 5 ng/ml rhFLT3L, and 5 ng/ml rhIL-7. Medium was changed completely every 3-4 days. After differentiation culture for 5-7 weeks, the generated CAR T cells or T cells were harvested by adding MACS buffer (PBS/0.5% bovine serum album/2 mM EDTA) to each well and briefly dis-aggregating the culture by pipetting with a 1 mL pipet, followed by passage through a 50 μm nylon strainer.

To differentiate iPSC into HSPC then further differentiate into CAR T or T cells, 250 uL cell mixture containing 2.5×10⁵ enriched CD34+HSPC cells and 5×10⁶ irradiated MS-hDLL4 cells were directly seeded on a nanofiber matrix insert in a 6 well plate with 2 ml RB27 medium with 5 ng/ml rhFLT3L and 5 ng/ml rhIL-7. Medium was changed completely every 2-3 days for 5-7 weeks.

The nanofiber matrix based co-culture was also set up by directly adding hMEP/HSPC cells and irradiated MS5-DLL4 cell suspension in nanofiber plates (Nanofiber solutions) and centrifuging at 300 g for 3 min.

The nanofiber matrix based co-culture were prepared by mixing EMP/HSPC and MS5-DLL4 cells with micronized nanofibers in RB27 medium or methylcellulose based semi-solid medium, followed by seeding into ultralow attachment plates.

Differentiation CAR+/− iPSC into CAR T/T Cells by Nanofiber Matrix Based Culture System (Protocol 3B)

iPS cells with or without CAR expression were induced to differentiate into CAR T or T cells by a similar protocol with protocol 3A (above) with modification. Briefly, the feeder cells in 3B would use MS5_DLL1 as feeder cells instead of MS5_DLL4.

Differentiation from iPSC directly to CAR NK cells or NK cells, 10 ng/mL IL15 was added along with other cytokines (10 ng/ml rhSCF, 5 ng/ml rhFLT3L, and 5 ng/ml rhIL-7) from day 0.

Differentiation from iPSC to HSPC then to CAR NK cells or NK cells, 10 ng/ml rhSCF and 10 ng/ml IL15 was supplied in RB27 along with other cytokines (5 ng/ml rhFLT3L, and 5 ng/ml rhIL-7).

CAR T Cell Preparation

PBMC or T_{n/mem} were stimulated with Dynabeads Human T expander CD3/CD28 (Invitrogen) at a ratio of 1:3 (T cell:bead) and transduced with lentivirus to express CAR in X-VIVO 15 (Lonza) medium containing 10% FCS with 20 μg/ml protamine sulfate (APP Pharmaceuticals), 50 U/ml recombinant human IL-2 (rhIL-2), and 0.5 ng/ml rhIL-15. Cultures were then maintained at 37° C., 5% CO2 under the same condition of media and cytokines. Cytokines were supplied every other day. On day 7 after transduction, the CD3/CD28 Dynabeads were removed from cultures using the DynaMag-50 magnet (Invitrogen).

Flow Cytometry

iPSC cells were dissociated with Accutase (Innovative Cell Technologies) and resuspended in mTeSR1 medium with 1X CloneR supplement (Stemcell Technologies). T cells were harvested and stained as described previously {Jonnalagadda, 2015 #3; Jonnalagadda, M., et al., Chimeric antigen receptors with mutated IgG4 Fc spacer avoid fc receptor binding and improve T cell persistence and antitumor efficacy. Mol Ther, 2015. 23(4): p. 757-68}. iPSC phenotype was examined using fluorochrome-conjugated antibodies against SSEA3, SSEA4, TRA1-60, TRA1-81, CD30. T cell phenotype was examined using fluorochrome-conjugated antibodies against CD3, CD4, CD8a, CD8b, CD5, CD7, CD45, CD45RA, CD45RO, TCRab, TCRgd, CD16, CD56, CD27, CD28, NKP44, NKP46, NKG2A, NKG2D, CD178 (FasL), CD19. Transgene expression was determined by staining for the truncated EGFR or truncated CD19. Memory-associated phenotypes were analyzed with fluorochrome-conjugated antibodies against CD45RO, CD45RA, CD62L. All samples were analyzed via a MacsQuant Analyzer (Miltenyi Biotec) and processed via FlowJo v10.

TCR Vβ Repertoire Expression Analysis

T cell receptor Vβ staining was determined using three-color flow cytometry with the IOTest Beta Mark TCR Repertoire Kit (Beckman Coulter) which consists of monoclonal antibodies (mAbs) designed to identify 24 distinct TCR Vβ families. Each set consisted of three distinct anti-Vβ family-specific mAb labelled with fluorescein isothiocyanate (FITC), phycoerythrin (PE), or doubly labelled with FITC and PE. T cell population was also co-stained with APC-anti-CD3 antibody and CD3+ population was gated for analysis.

In Vitro T Cell Assays

To test for cytotoxicity and activity, target tumor cells were planted in 96 round bottom well plates at indicated density. T cells were then washed and resuspended in the same media and added to the target cells. To test for degranulation, CAR T or control T cells were incubated with target cells for 5 hours in the presence of CD107a antibody and Golgistop protein transport inhibitor (BD Biosciences). After the co-culture, cells were harvested, fixed, permeabilized, and stained for intracellular cytokines. Degranulation (CD107a staining) and intracellular cytokine staining were examined by flow cytometry. For cytotoxicity tests, co-culture would last 4 hour for short term assay and 48 hour for long term assay as indicated. After co-culture, all cells were harvested and stained with indicated antibodies, followed by quantification by flow cytometry.

In Vitro Cytokine Production Assay

CAR T or T cells were co-incubated for 24 hours with different target cells at an effector-to-target (E:T) ratio of 1:1. Supernatant was collected and the cytokines were examined by cytokine 10-plex human panel kit (Invitrogen) with Bio-Plex reader (Bio-Rad).

In Vivo Xenograft Studies

All mouse experiments were approved by the COH IACUC. Tumor xenograft models were generated using 6- to 8 week-old NOD/SCID/IL2R−/− (NSG) mice as previously described (e.g. Urak, R., et al., Ex vivo Akt inhibition promotes the generation of potent CD19CAR T cells for adoptive immunotherapy. J Immunother Cancer, 2017. 5: p. 26). Briefly, on day 0, ffLuc+NALM6 cells (1×10⁶) were intraperitoneal injected (i.p.) into the NSG mice. After 4 days, mice were then treated intraperitoneally with CAR T cells or T cells as indicated for each experiment. Tumor growth was determined by in vivo biophotonic imaging using a Xenogen IVIS 100. Mice were also monitored for survival, with euthanasia applied according to the American Veterinary Medical Association Guidelines.

### Methods for Example 4

Certain reagents and resources used in Example 4 are described in a table at the end of this section.

Mice

All animal experiments were conducted under a protocol approved by City of Hope Animal Research Committee. This study used 6-8 week-old NOD.Cg-Prkdc^{scid} Il2rg^{tm1Wjl}/SzJ (NSG) mice from Jackson Laboratory.

DNA Constructs

The CD19-targeted chimeric antigen receptor (CD19-CAR) construct is the same as currently used in our clinical studies targeting B cell leukemia/lymphoma (clinicaltrials.gov #NCT02146924) [1, 2]. The CD19-CAR contains an anti-CD19 scFv domain derived from the FMC63 mAb [3], an IgG4 Fc spacer with two point mutations (L235E and N297Q) within the CH2 region, a CD28 transmembrane domain, a CD28ζ costimulatory domain, and a CD3 signaling domain. A T2A ribosome skip sequence [4] then separates this CAR sequence from a truncated human epidermal growth factor receptor sequence (huEGFRt) which can be used as a selection marker and safety switch[5-7]. The episomal plasmids encoding OCT3/4/shp53, SOX2/KLF4, L-MYC/LIN28, and EBNA were gifts from Shinya Yamanaka[8].

Tn/mem Isolation

Blood products were obtained from healthy donors under protocols approved by the COH IRB, and naïve and memory T (Tn/mem) cells were isolated following similar procedures described in previous studies[9]. In brief, human peripheral blood mononuclear cells (PBMC) were isolated by density gradient centrifugation over Ficoll-Paque (GE Healthcare) and then underwent sequential rounds of CliniMACS/AutoMACS (Miltenyi Biotec) depletion to remove CD14- and CD25-expressing cells, followed by a CD62L-positive selection for Tn/mem cells.

Generation of iPSCs from Tn/mem

Tn/mem cells were reprogrammed into pluripotent stem cells (iPSCs) by an integration-free method modified from a published protocol N. In brief, one million Tn/mem cells were electroporated with 3 μg of plasmid mixture using the Human T Cell Nucleofector Kit and the Nucleofector 4D electroporation device (Lonza). The plasmid mixture was composed of episomal plasmids encoding OCT3/4, SOX2, KLF4, L-MYC, LIN28, and shRNA for TP53 N. The transfected cells were cultured in X-VIVO 15 medium (Lonza) supplemented with 10% FBS (HyClone), 50 U/mL rhIL-2 (Novartis Oncology), 0.5 ng/mL rhIL-15 (CellGenix) and Dynabeads Human T-Expander CD3/CD28 (ThermoFisher Scientific) (bead to cell ratio of 1:1). Two days after the transfection, an equal volume of pluripotent stem cell (PSC) medium containing rhFGF-basic and 10 μM Y27632 was added [8]. The medium was then completely changed to PSC medium 4 days after transfection. iPSC colonies were visible at day 20-30 and individual colonies were picked under a microscope for further culture/expansion in cGMP-grade mTeSR1 medium (StemCell Technologies) in Matrigel-coated (Corning) plates.

Generation of CAR-Positive, Clonal iPSC Lines

Before lentivirus transduction, iPSC cultures were dissociated with Accutase (ThermoFisher Scientific) treatment and the cells were seeded at a density of 10⁵ per well in 12-well plates in mTeSR1 medium supplemented with 1X CloneR and 10 μM ROCK inhibitor Y-27632 dihydrochloride (StemCell Technologies). After overnight culture, cGMP lentivirus encoding CD19-CAR was added to the culture with 10 μg/mL protamine sulfate (APP Pharmaceuticals) to transduce the iPSCs (multiplicity of infection [MOI]=1). The transduced cells were cultured for at least two passages before single cell sorting by flow cytometry and iPSC colonization. Clonal CAR-positive cells were again expanded in mTeSR1 medium on Matrigel-coated plates, and banked for subsequent differentiation.

Integration Detection by PCR

EBNA1 is a common component of all episomal vectors [10]. To detect genomic integration of episomal plasmids used for iPSC reprogramming from T cells, PCR was performed to amplify integrated EBNA components from genomic DNA using primers as follows: EBNA1_For: ATCAGGGCCAAGACATAGAGATG, EBNA1_Rev: GCCAATGCAACTTGGACGTT. Plasmid integration free iPSC clones did not show EBNA1 signal. FBX15, which was expressed on pluripotent stem cells, was used as house-keeping gene here and was amplified by the following primers: FBX15_For: GCCAGGAGGTCTTCGCTGTA; FBX15 Rev: AATGCACGGCTAGGGTCAAA.

Teratoma Formation Assay

Two million dissociated iPSCs were suspended in 200 uL medium (100 uL PBS (Irvine Scientific) and 100 uL Matrigel) and injected subcutaneously into NSG mice. After 5-8 weeks, teratomas were harvested in PBS, fixed overnight in 4% paraformaldehyde (Boston BioProducts) at room temperature, and maintained thereafter in 70% ethanol for processing. Samples were submitted to the City of Hope Histology Core Facility for sectioning and hematoxylin and eosin staining. Sections were examined, interpreted, and photographed microscopically.

Differentiation of Tn/mem-Derived, CAR+ iPSC into CAR+ T Cells by PSC-ATO Culture

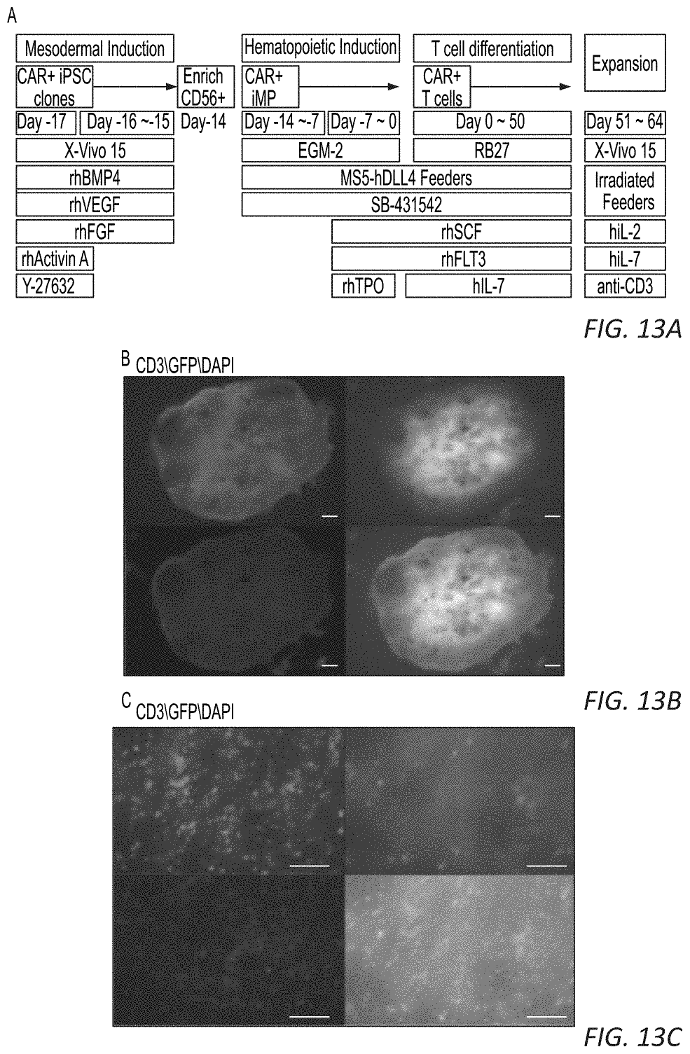

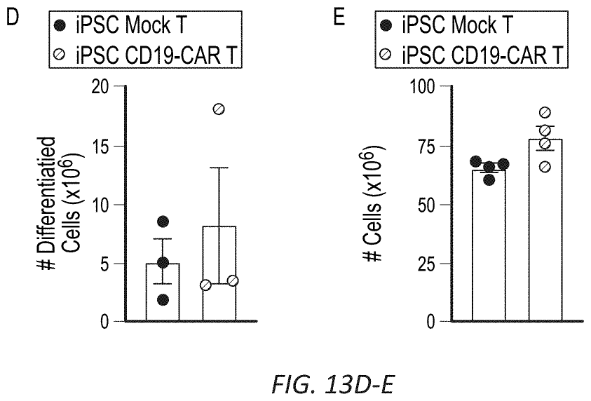

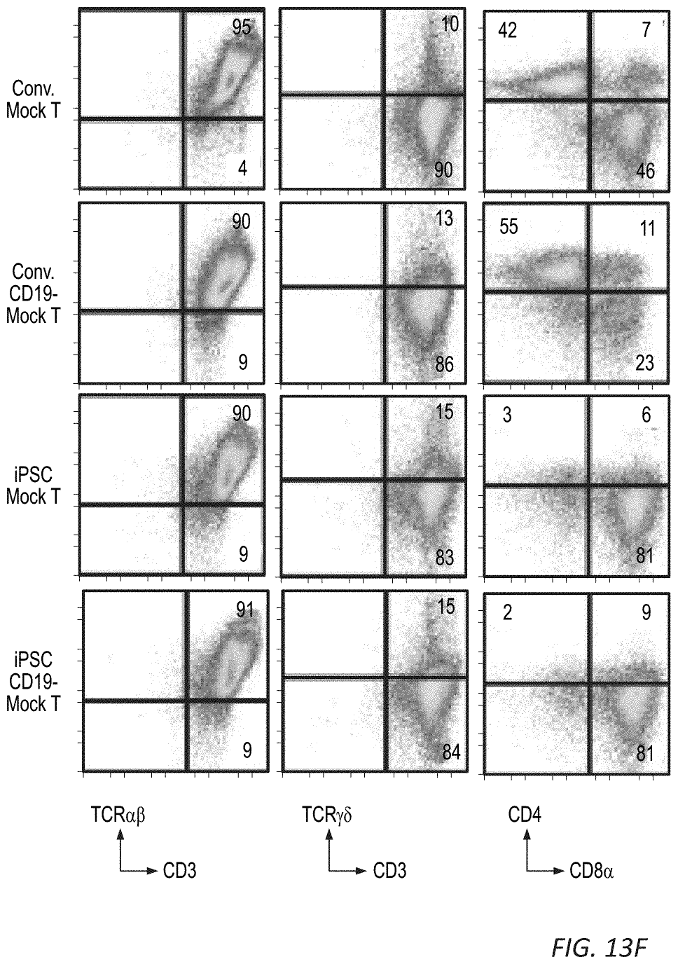

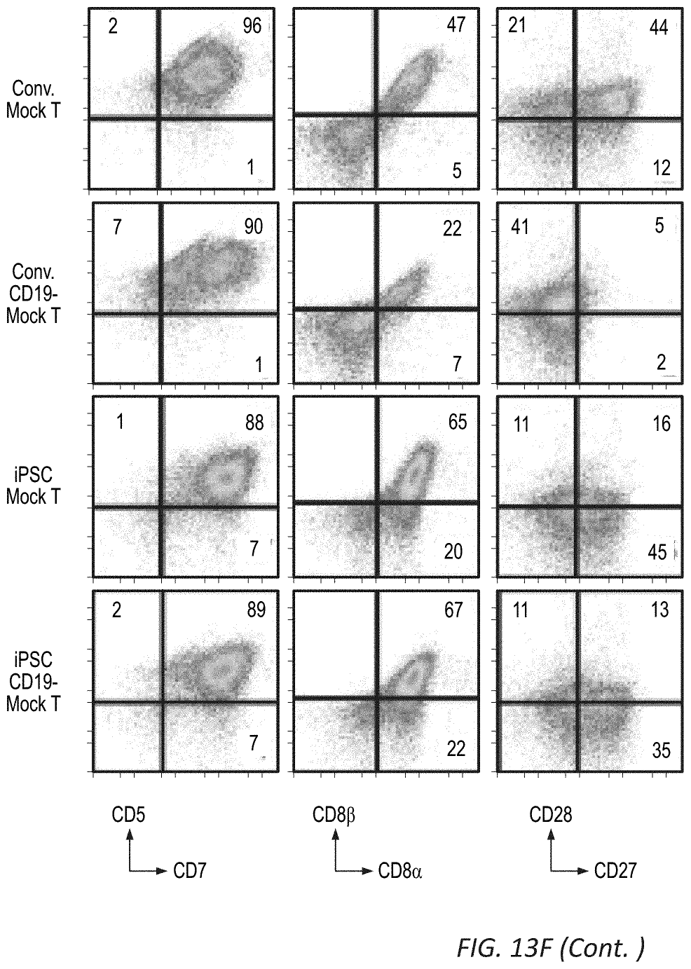

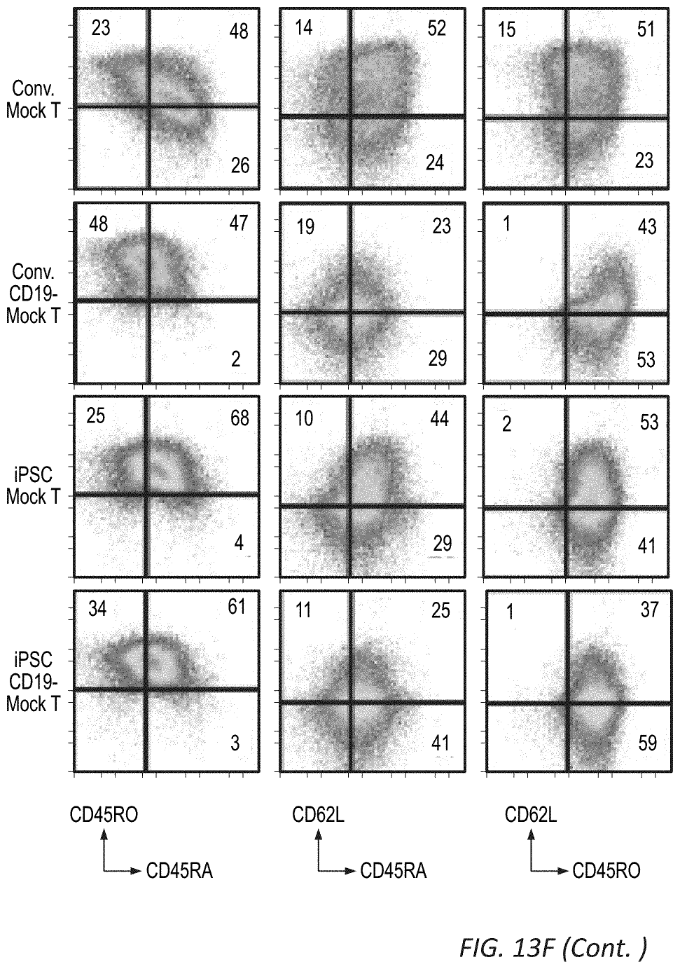

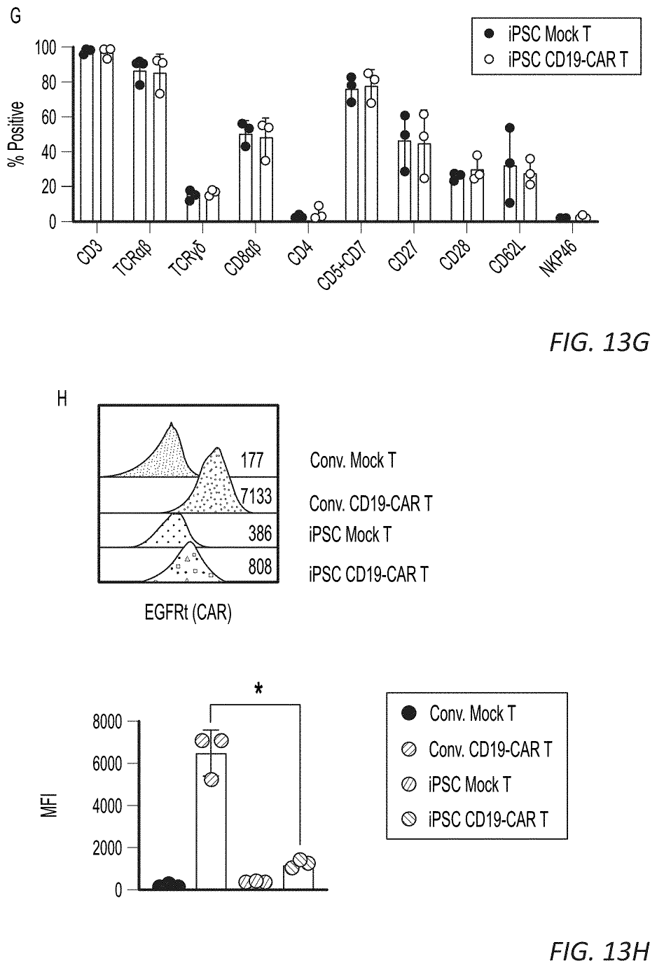

The schema of the sequential differentiation protocol is outlined in FIG. 13A. First, mesoderm commitment was induced as previously described [11-13]. Briefly, iPSCs were harvested as a single cell suspension after Accutase treatment, resuspended at 1×10⁶ cells/mL in X-VIVO 15 medium containing 10 ng/mL rhActivin A (R&D Systems), 10 ng/mL rhBMP4 (R&D Systems), 10 ng/mL rhVEGF (R&D Systems), 10 ng/mL rhFGF (Peprotech), and 10 μM ROCK inhibitor Y-27632 dihydrochloride (StemCell Technologies). Three million cells per well were plated in Matrigel-coated 6-well plates. Medium was then changed daily with X-VIVO 15 containing 10 ng/mL rhBMP4, 10 ng/mL rhVEGF, and 10 ng/mL rhFGF. Three days later (Day −14 in FIG. 13A), cells were washed 3 times with PBS (Irvine Scientific) and incubated with 1 mL per well Accutase for 5-7 minutes at 37° C. Cells were harvested, washed in PBS containing 1 mM EDTA and 2% FBS, and CD56+CD326− human iPSC mesodermal progenitors (iMP) were isolated by CD56 enrichment using EasySep Positive Selection kits (StemCell Technologies). Flow cytometry was performed to confirm CD56+CD326− phenotype of the iMP.

iPSC mesodermal organoids (iMOs) were generated by aggregating iMP cells and MS5-hDLL4 feeder cells. On day −14, MS5-hDLL4 cells were harvested with trypsin and washed into hematopoietic induction medium composed of EGM-2 (Lonza) with 10 μM Y-27632 and 10 μM TGF-βRI inhibitor SB-431542 (StemCell Technologies). After using a 40 μm nylon mesh strainer to remove aggregates, 5×10⁵ MS5-hDLL4 cells were combined with 0.5-1×10⁴ purified iMP cells in 1.5 mL microfuge tubes and centrifuged at 300×g for 5 min at 4° C. in a swinging bucket centrifuge. Up to 12 iMOs were prepared in each tube. After carefully removing the supernatant, the MS5-hDLL4/iMP cell pellet was resuspended by brief pulse vortexing in hematopoietic induction medium (i.e., EGM-2 with 10 μM SB-431542) at 6 μl per iMO. Two 6 μL aliquots of cells were plated as iMOs on one Millicell transwell insert (Millipore Sigma) per well in 6-well plates containing 1.5 mL hematopoietic induction medium. Medium was changed completely every 2-3 days for one week. On day −7, medium was changed to EGM-2 with 10 μM SB-431542 plus 5 ng/mL rhTPO (Peprotech), 5 ng/mL rhFLT3L (Peprotech), and 50 ng/mL rhSCF (Peprotech).

On day 0, the artificial thymic organoid (ATO) T cell differentiation phase was initiated with a switch to serum-free ATO culture medium containing 10 ng/mL rhSCF, 5 ng/mL rhFLT3L, and 5 ng/mL rhIL-7 in RB27 medium that consisted of RPMI 1640 (Lonza), with 4% B27 Supplement (ThermoFisher Scientific), 30 μM L-ascorbic acid 2-phosphate sesquimagnesium salt hydrate (Sigma), 1% GlutaMAX (ThermoFisher Scientific), 1% Penicillin-Streptomycin (Lonza), 55 uM 2-mercaptoethanol (ThermoFisher Scientific), and 1% MEM Non-essential Amino Acids (ThermoFisher Scientific). Medium was changed completely every 2-3 days. After 5-7 weeks of differentiation, iMO-ATO-derived CAR+ T cells were harvested by pipetting 1-2 mL of X-VIVO 15 with 10% FBS onto the surface of each transwell insert and disaggregating the iMO-ATO by repeated aspiration with a P1000 pipettor. Single cells were isolated by passing the disaggregated cell suspension through a 40 μm nylon mesh strainer. An aliquot of the recovered cells was stained with the indicated antibodies for phenotyping by flow cytometry and the remaining cells were cultured in a previously described rapid expansion method (REM) [14, 15], with minor modifications. Briefly, 1×10⁶ iMO-ATO-derived T cells were combined with 50×10⁶ γ-irradiated (35 Gy) PBMCs and 10×10⁶ γ-irradiated LCL cells (80 Gy) in 50 mL X-VIVO 15 medium containing 10% FBS, 20 ng/mL anti-CD3 (Miltenyi Biotec), 50 U/mL rhIL-2 and 10 ng/mL rhIL-7. REM cultures were maintained for 14 days, with half-volume medium changes every 48 hours.

Immunohistochemistry

The PSC-ATO organoids were fixed and permeabilized with the Fixation/Permeabilization Solution Kit (BD Biosciences), stained with PE-anti-CD3 and DAPI in permeabilization buffer for 15 minutes and then rinsed with wash buffer three times. In situ images were taken with a BZ-X810 fluorescence microscope (Keyence).

Generation of Conventional CD19-CAR T Cells

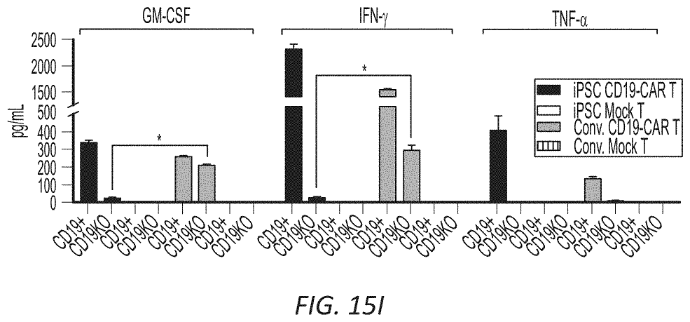

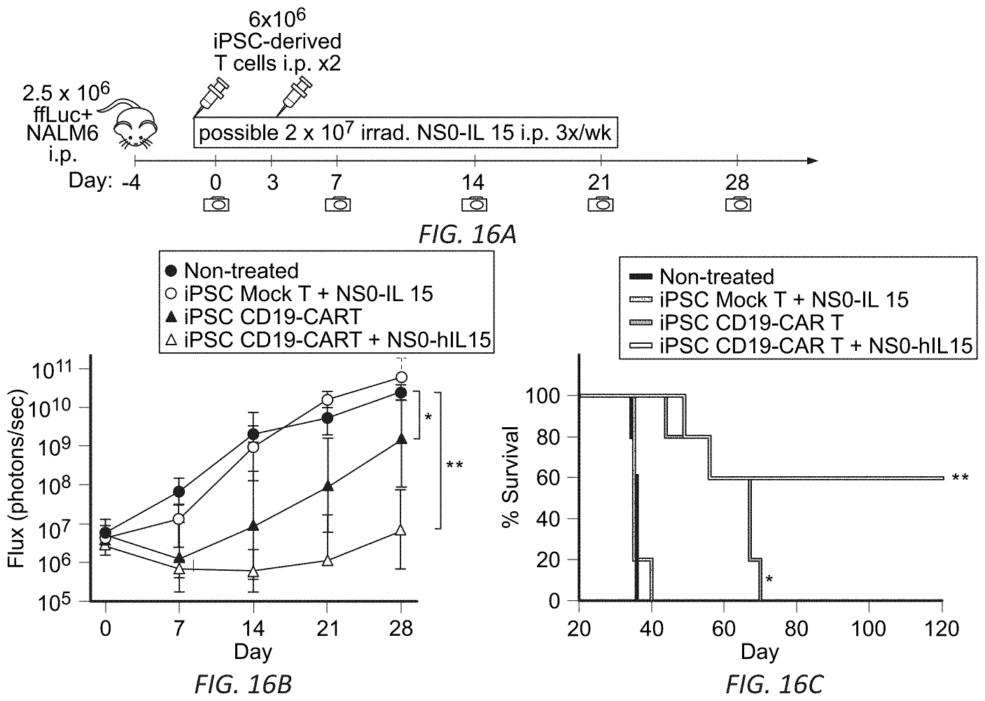

PBMC (or Tn/mem as indicated for FIG. 16D-F only) were stimulated with Dynabeads Human T-Expander CD3/CD28 at a ratio of 1:2 (cells:beads) in X-VIVO 15 medium containing 10% FBS, 50 U/mL rhIL-2, and 0.5 ng/mL rhIL-15. Cells were transduced with clinical grade lentivirus to express CD19CAR with 25 μg/mL protamine sulfate (APP Pharmaceuticals). Cultures were then maintained at 37° C., 5% CO₂ under the same medium and cytokine conditions. Fresh cytokines were supplied every other day. On day 7 after transduction, the CD3/CD28 Dynabeads were removed from cultures using the DynaMag-50 magnet (ThermoFisher Scientific). The cells were expanded in culture until harvest at day 17 or as indicated. The PBMC-derived, CAR+ T cells were enriched by EasySep kit with anti-EGFRt antibody (StemCell Technologies) and used for phenotype characterization and functional assays; Tn/mem-derived CAR+ T cells used in the in vivo assays were not enriched, but dosed based on CAR+.

Flow Cytometry

iPSCs were dissociated with Accutase (ThermoFisher Scientific) and resuspended in mTeSR1 medium with 1X CloneR supplement (StemCell Technologies). iPSC phenotype was examined using fluorochrome-conjugated antibodies against EGFR (to detect transgene), SSEA3, SSEA4, TRA1-60, TRA1-81, and CD30. T cells were harvested and stained as described previously[5]. T cell phenotype was examined using fluorochrome-conjugated antibodies against CD3, CD4, CD8α, CD8β, CD5, CD7, TCRαβ, TCRγδ, CD16, CD56, CD27, CD28, NKP44, NKP46, NKG2A, NKG2D, CD178 (FasL), and CD19. CAR expression was determined by staining for the truncated EGFR. Memory-associated phenotypes were evaluated with fluorochrome-conjugated antibodies against CD45RO, CD45RA, and CD62L.

T cell receptor Vβ staining was performed with the IOTest Beta Mark TCR Repertoire Kit (Beckman Coulter) which consists of monoclonal antibodies (mAbs) designed to identify 24 distinct TCR Vβ families. Each set consisted of three distinct anti-Vβ family-specific mAbs labelled with fluorescein isothiocyanate (FITC), phycoerythrin (PE) or doubly labelled with FITC and PE. The T cell population was also co-stained with APC-anti-CD3 antibody and the CD3-positive population was gated on for analysis. Data were acquired on MacsQuant Analyzer 10 (Miltenyi Biotec) or Fortessa (Becton Dickinson) flow cytometers and analyzed with FlowJo (v10.6.1).

PCR Based TCR β Clonality Assay

Genomic DNA was extracted by DNeasy kit (Qiagen) and used as PCR template. The PCR assay was set up according to the protocol of IdentiClone TCRB+TCRG T-Cell Clonality Assay Kit (Invivoscribe) [16, 17], TCRB tube A and B primer master mix target framework regions within the variable region and joining region of the TCR beta chain locus. TCRB Tube C targets the diversity and joining regions of the TCR beta chain locus. The specimen control size ladder master mix targets multiple genes and generates a series of amplicons to serve as quality control of input DNA. The primers are fluorescence labelled and fragment analysis was performed to detect the fragment size of PCR products concomitantly with regular DNA agarose gel examination.

In Vitro T Cell Functional Assays

Effector cells (iPSC CD19-CAR T, iPSC Mock T, conventional CD19-CAR T or conventional Mock T cells) were washed, resuspended in fresh medium containing 50 U/mL rhIL-2 and 0.5 ng/mL rhIL-15 and co-cultured in 96-well U-bottom plates with the indicated tumor cells at the indicated effector-to-target (E:T) ratios for 4 hours or 48 hours. Cytotoxic activity was then routinely evaluated by flow cytometry by enumerating viable (i.e., DAPI-negative) GFP-expressing tumor cells; for primary ALL cells, DAPI-/CD19+ cells were enumerated. Alternatively, for luciferase based cytotoxicity assays, at each timepoint, D-luciferin potassium salt (PerkinElmer) was added to each well at a final concentration of 0.14 mg/mL and plates were incubated at 37° C. for 10 minutes.

Following the incubation with luciferin, the contents of each culture plate were mixed carefully and transferred to an opaque 96-well U-bottom plate with a multichannel pipettor. Bioluminescent flux was read with a Cytation 3 plate reader (Biotek). For each tumor line, replicate wells of tumor cells alone were used to generate internal MIN (0% viability) and MAX (100% viability) references for the calculation of percent lysis; the MIN was obtained by the addition of SDS to a final concentration of 1% ten minutes before the addition of luciferin [18].

To evaluate T cell activation, iPSC-derived or conventional CAR T or Mock T cells were incubated with the indicated tumor cells at an E:T ratio of 1:1 for five hours in the presence of CD107a antibody and GolgiStop protein transport inhibitor (BD Biosciences). Cells were then harvested, fixed, permeabilized, and stained for intracellular cytokines. Degranulation (CD107a staining) and intracellular cytokine staining (e.g. IFNγ) on CD3-gated cells was then examined by flow cytometry. Similar co-cultures without GolgiStop were harvested for staining of surface activation markers CD25 and CD137/4-1BB on CD3-gated cells was evaluated by flow cytometry.

To further characterize cytokine production, iPSC-derived or conventional CAR T or Mock T cells were co-incubated for 24 hours with the indicated NALM6 tumor cells at an E:T ratio of 1:1 in medium without added cytokines. Supernatants were collected and cytokine levels were quantified with the Cytokine 10-Plex Human Panel Kit (ThermoFisher Scientific) by a Bio-Plex reader (Bio-Rad). Similar co-cultures were harvested for flow cytometric analysis of surface activation markers CD25 and CD137/4-1BB on CD3-gated cells.

For In vitro repetitive challenge assay, 10⁵ CAR T cells were co-cultured with 4×10⁵ CD19+ NALM6 cells at E:T ratio of 1:4, and re-challenged every other day with 4×10⁵ NALM6 cells for 3 times. The cells were then stained with surface exhaustion markers PD-1, TIM-3 and LAG-3 together with T cells markers. Each of exhaustion marks was evaluated on CD3-gated cells by flow cytometry[19].

RNA and Protein Analysis

RNA was extracted with the Quick-RNA Microprep kit (Zymo Research) and treated with DNase I. RNA deep sequencing was performed by the City of Hope Integrative Genomics Core Facility. Briefly, stranded RNA-seq libraries were prepared using the KAPA mRNA HyperPrep kit (Roche), according to the manufacturer's recommended protocol. Libraries were quantified using Qubit quantification kit (Thermofisher Scientific) and loaded onto the HiSeq 2500 sequencing platform (Illumina) for single-end 51-bp sequencing. Base calling was done using Illumina Real Time Analysis (RTA) v1.18.64.

For protein analysis by western blot, the harvested cells were lysed in RIPA buffer (ThermoFisher Scientific) and protein extraction was quantified with a BCA protein assay kit (ThermoFisher Scientific). The Bolt Mini Gel System (ThermoFisher Scientific) was used for gel electrophoresis and protein transfer. Anti-p44/42 MAPK (Erk1/2) and anti-phospho-p44/42 MAPK (Erk1/2) (Thr202/Tyr204); anti-PLCγ1, anti-phospho-PLCγ1 (Tyr783) and anti-phospho-PLCγ1 (Ser1248); anti-CD3, and anti-phospho-CD3ζ(Y142); and anti-phospho-ZAP70 were used to interrogate CAR T and T cell signaling pathways (see Resource Table for antibody details).

Bioinformatics Analysis of RNA Seq Data

To analyze the RNA seq data, the 2-D visualization of PCA was implemented using R package “DESeq2” (v.3.10) based on the PCA algorithm. Heatmaps of z-scores were generated by Cluster (v.3.0) and JavaTreeView (v.1.1.6r4) using a hierarchical clustering approach. Differentially expressed gene (DEG) analysis was performed with R package “edgeR” (v.3.28.0)[20]. The pipelines of deriving DEG involved the quantile-adjusted conditional maximum likelihood (qCML), and the quasi-likelihood (QL) F-test. Bubble plots were acquired with R package “ggplot2” (v.3.2.1). The Gene Set Enrichment Analysis (GSEA) algorithm was run on GSEA (v.4.0.3) [2, 22]. The resources of bioinformatic software packages are listed in the table of ‘Key resources’.

Bisulfite Conversion, PCR, and Sequencing

Genomic DNA was prepared by DNeasy kit (Qiagen). 500 ng genomic DNA was treated with sodium bisulfite to convert unmethylated cytosines using the EZ DNA Methylation-Lightning Kit (Zymo Research). Reactions were carried out per manufacture's protocol. Methylation-specific PCR was performed, and 245 bp PCR fragments of EF1a promoter from bisulfite converted gDNA of iPSC CD19-CAR T cells and conventional CD19-CAR T cells were amplified. The PCR fragments were subcloned into a pCR4-TOPO vector (Thermo Fisher Scientific) and six clones of each group were sequenced by Sanger Sequencing. The sequencing results were aligned to original and putative methylated sequences to determine the methylation status of CG sites.

Animal Studies

All mouse experiments were conducted with protocols approved by City of Hope Institutional Animal Care and Use Committee. Tumor xenograft models were generated using 6 to 8 week-old NOD/SCID/IL2Rγ−/− (NSG) mice as previously described (Jackson Laboratory)[6]. Briefly, on day 0, ffLuc⁺ NALM6 cells (2.5×10⁵) were injected either intraperitoneally (i.p.) or intravenously (i.v.) into the NSG mice. After 4 days, mice were then treated with iPSC-derived or conventional CAR T or Mock T cells as described for each experiment. Mice in the indicated groups were injected i.p. three times per week with 20×10⁶ irradiated (80 Gy) human hIL-15-secreting nurse cells (IL15-NS0) [14]. Reference schematics of FIGS. 16A and 16D for injections of T cells in each tumor model. Tumor growth was determined weekly by in vivo biophotonic imaging using a Xenogen IVIS 100. Mice were also monitored for survival, with euthanasia applied according to the American Veterinary Medical Association Guidelines.

| REAGENT or RESOURCE | SOURCE | IDENTIFIER |
|---|---|---|
| Antibodies | | |
| Anti-human SSEA-3 | BD Biosciences | (clone MC-6310) RRID:AB_1645542 |
| Anti-human SSEA-4 | BD Biosciences | (clone MC813-70) RRID:AB_2033991 |
| Anti-human Tra-1-60 | BD Biosciences | (clone TRA-1-60) RRID:AB_10565983 |
| Anti-human TRA-1-81 | BD Biosciences | (clone TRA-1-81) RRID:AB_1645493 |
| Anti-human EpCAM (CD326) | BioLegend | (clone 9C4) RRID:AB_756082 |
| Anti-human CD56 | BD Biosciences | (clone NCAM16.2) RRID:AB_400025 |
| Anti-human CD30 | BD Biosciences | (clone BerH8) RRID:AB_393541 |
| Anti-human CD3 (BV510) | BD Biosciences | (clone UCHT1) RRID:AB_2732053 |
| Anti-human CD3 (APC) | BD Biosciences | (clone SK7) RRID:AB_400513 |
| Anti-human CD4 (Per-CP) | BD Biosciences | (clone SK3) RRID:AB_400282 |
| Anti-human CD4 (PE-Cy7) | BD Biosciences | (clone SK3) RRID:AB_396897 |
| Anti-human CD8α (PE) | BD Biosciences | (clone SK1) RRID:AB_400005 |
| Anti-human CD8α (Per-CP) | BD Biosciences | (clone SK1) RRID:AB_400280 |
| Anti-human CD8α (APC-Cy7) | BD Biosciences | (clone SK1) RRID:AB_400383 |
| Anti-human CD8β | BD Biosciences | (clone 2ST8.5H7) RRID:AB_1645747 |
| Anti-human CD5 | BD Biosciences | (clone UCHT2) RRID:AB_2737700 |
| Anti-human CD7 | BD Biosciences | (clone M-T701) RRID:AB_2738544 |
| Anti-human TCR αβ | BD Biosciences | (clone T10B9.1A-31) RRID:AB_2738437 |
| Anti-human TCR γδ | BD Biosciences | (clone B1) RRID:AB_396061 |
| Anti-human NKG2A | R&D Systems | (clone 131411) RRID:AB_2132978 |
| Anti-human NKG2D (CD314) | BD Biosciences | (clone 1D11) RRID:AB_10896282 |
| Anti-human NKp44 (CD336) | BD Biosciences | (clone p44-8) RRID:AB_647239 |
| Anti-human NKP46 (CD335) | BioLegend | (clone 9E2) RRID:AB_2561649 |
| Anti-human CD19 | BD Biosciences | (clone SJ25C1) RRID:AB_396893 |
| Anti-human CD16 | BD Biosciences | (clone NKP15) RRID:AB_400317 |
| Anti-human Fas L (CD178) | BD Biosciences | (clone NOK-1) RRID:AB_2738713 |
| Anti-human CD62L (PE) | BD Biosciences | (clone SK11) RRID:AB_400205 |
| Anti-human CD62L (FITC) | BD Biosciences | (clone SK11) RRID:AB_400302 |
| Anti-human CD45RA | BD Biosciences | (clone HI100) RRID:AB_398468 |
| Anti-human CD45RO | BD Biosciences | (clone UCHL1) RRID:AB_395883 |
| Anti-human EGFR (APC) | BioLegend | (clone AY13) RRID:AB_11150410 |
| Anti-human EGFR (PE-Cy7) | BioLegend | (clone AY13) RRID:AB_2562159 |
| Anti-human CD28, InVivoMab | Bio X Cell | (clone 9.3) RRID:AB_2687729 |
| Anti- human CD27 | BD Biosciences | (clone M-T271) RRID:AB_395834 |
| Anti-human CD28 (APC-H7) | BD Biosciences | (clone CD28.2) RRID:AB_11154032 |
| Anti-human CD279 (PD-1) | ThermoFisher Scientific | (clone eBioJ105) RRID:AB_2573976 |
| Anti-human LAG3/CD223 | Lifespan | (clone 17B4) RRID:AB_1650048 |
| Anti-human TIM-3 | R&D Systems | (clone 344823) RRID:AB_2232901 |
| Pan p44/42 MAPK (phosphorylated | Cell Signaling | polyclonal |
| Erk1/2) antibody | Technology | |
| Pan p44/42 MAPK (Erk1/2) antibody | Cell Signaling | (clone 3A7) RRID:AB_10695739 |
| | Technology | |
| Anti-human, -mouse phospho-PLCγ1 | Cell Signaling | (clone D6M9S) RRID:AB_2728690 |
| (Tyr783) antibody | Technology | |
| Anti-human, -mouse, -monkey | Cell Signaling | (clone D25A9) RRID:AB_10890863 |
| phospho-PLC 1 (Ser1248) antibody | Technology | |
| Anti-human, -rat, -mouse PLCγ1 | Cell Signaling | polyclonal |
| | Technology | |
| Anti-human, -mouse phospho-Zap-70 | Cell Signaling | (clone 65E4) RRID:AB_2218658 |
| (Tyr319)/Syk (Tyr352) | Technology | |
| Anti-human, -mouse, anti-CD3ζ | BD Biosciences | (clone K25-407.69) RRID: AB_647307 |
| (pY142) | | |

Chemicals, Peptides, and Recombinant Proteins

| | | |
|---|---|---|
| Proleukin (aldesleukin), rhIL-2 | Novartis Oncology | NCD 65483-116-07 |
| rhIL-15 | CellGenix, US Operations | 1413-050 |
| Activin A | R&D Systems | 338-AC-010 |
| rhBMP-4 | R&D Systems | 314-BP-010 |
| rhVEGF | R&D Systems | 293-VE-010 |
| rhFGF-basic | Peprotech | AF-100-18B |
| ROCK inhibitor, Y-27632 | StemCell Technologies | 72304 |
| dihydrochloride | | |
| TGFβ-RI inhibitor, SB-431542 | StemCell Technologies | 72234 |
| rhFlt3-Ligand | Peprotech | AF-300-19 |
| rhIL-7 | Peprotech | AF-200-07 |
| rhTPO | Peprotech | 300-18 |
| rhSCF | Peprotech | AF-300-07 |
| MEM α, with nucleosides | GIBCO | 12571063 |
| Fetal bovine serum, defined, heat- | HyClone | SH30070.03IH |
| inactivated | | |
| X-VIVO 15 Serum-free Hematopoietic | Lonza | BEBP04-744Q |
| Cell Medium | | |
| EGM-2, Endothelial Cell Growth | Lonza | CC-3162 |
| Medium-2 Bullet Kit | | |
| RPMI 1640 Medium with L-Glutamine | Lonza | 12-115F |
| and HEPES | | |
| GLutaMAX Supplement | ThermoFisher Scientific | 35050061 |
| 2-Mercaptoethanol, 55 mM | ThermoFisher Scientific | 21985023 |
| MEM Non-Essential Amino Acids | ThermoFisher Scientific | 11140050 |
| Solution (100X) | | |
| L-Ascorbic acid 2-phosphate | Sigma | A8960 |
| sesquimagnesium salt hydrate | | |
| Phosphate-buffered saline | Irvine Scientific | 9240 |
| Penicillin-Streptomycin (100X) | Lonza | 17602E |
| mTeSR1 | StemCell Technologies | 85850 |
| DMEM/F12, with HEPES | ThermoFisher Scientific | 11330057 |
| Matrigel hESC-Qualified Matrix, LDEV- | Corning | 354277 |
| free | | |
| ReLeSR | StemCell Technologies | 5872 |
| CloneR | StemCell Technologies | 5889 |
| StemPro Accutase Cell Dissociation | ThermoFisher Scientific | A1110501 |
| Reagent | | |
| DNase I | Zymo Research | E1010 |
| B-27 Supplement (50X) | ThermoFisher Scientific | 17504-044 |
| TrypLE Express Enzyme (1X) | ThermoFisher Scientific | 12605010 |
| Protamine sulfate | APP Pharmaceuticals | NDC 63323-229-05 |
| XenoLight D-luciferin potassium salt | PerkinElmer | 122799 |
| Hanks' Balanced Salt Solution, w/o Ca⁺⁺, | GIBCO | 14175103 |
| Mg⁺⁺ | | |
| 0.05% Trypsin/0.53 mM EDTA in HBSS | Corning | MT25051CI |
| w/o Ca⁺⁺, Mg⁺⁺ | | |
| Plasmid constructs | | |
| CD19-CAR_pHIV (CD19-CAR) | Forman lab | N/A |
| hOCT3/4-shp53_pCXLE | addgene, gift from Shinya | RRID:Addgene_27077 |
| | Yamanaka | |
| hSK_pCXLE (SOX2, KLF4) | addgene, gift from Shinya | RRID:Addgene_27078 |
| | Yamanaka | |
| hUL_pCXLE (L-MYC, LIN28) | addgene, gift from Shinya | RRID:Addgene_27080 |
| | Yamanaka | |
| EBNA_pCXLE | addgene, gift from Shinya | RRID:Addgene_37624 |
| | Yamanaka | |
| Commercial Assays | | |
| Leukocyte Alkaline Phosphatase Kit | Sigma | 86R-1KT |
| IOTest beta Mark TCR Vβ Repertoire Kit | Beckman Coulter | IM3497 |
| TCRB T-Cell Clonality Assay Kit | Invivoscribe | 12000011 |
| CD34 MicroBead Kit UltraPure | Miltenyi Biotec | 130-100-453 |
| EasySep Human CD56 Positive Selection | Stem Cell Technologies | 17855 |
| Kit II | | |
| EasySep Human EpCAM Positive | Stem Cell Technologies | 17846 |
| Selection Kit II | | |
| Dynabeads ™ Human T-Expander | ThermoFisher Scientific | 11141D |
| CD3/CD28 | | |
| RNeasy Micro Kit | Qiagen | 74004 |
| Quick-RNA Microprep Kit | Zymo Research | R1050 |
| EZ DNA Methylation-Lightning Kit | Zymo Research | D5030 |
| Fixation/Permeabilization Solution Kit | BD Biosciences | 554714 |
| Brefeldin A Golgi Plug | BD Biosciences | 555029 |
| Luminex Cytokine Mouse Magnetic 10- | ThermoFisher Scientific | LMC0001M |
| Plex Panel | | |
| Millicell Cell Culture Insert, 30 mm, | Millipore Sigma | PICM0RG50 |
| hydrophilic PTFE, 0.4 μm | | |
| Experimental model: cell lines | | |
| iPSC lines | | N/A |
| MS5-hDLL4-eGFP | | N/A |
| NALM6.ffluc.eGFP | | N/A |
| NALM6.ffluc.eGFP_CD19KO | | N/A |
| Raji.ffluc.eGFP | | N/A |
| Raji.ffluc.eGFP_CD19KO | | N/A |
| 3T3_CD19 | | N/A |
| Experimental model: organisms | | |
| mouse:NSG:NOD. Cg-Prkdc^{scid} | The Jackson Laboratory | JAX: 005557 |
| IL2rg^{tm1Wjl}/SzJ | | |
| Software and Algorithms | | |
| GraphPad Prism GraphPad Software | GraphPad Software | www.graphpad.com/ |
| Flowjo | Tree Star Inc. | www.flowjo.com/solutions/flowjo |
| Zen | Zeiss | www.zeiss.com/microscopy/int/ |
| | | products/microscope-software/zen.html |
| BZ-X800 Analyzer | Keyence | www.keyence.com/landing/microscope/ |
| | | lp_fluorescence.jsp |
| R package “DESeq2” (v.3.10) | R | www.r-project.org |
| Cluster (v.3.0) | Human Genome Center | bonsai.hgc.jp/~mdehoon/software/ |
| (clustering algorithms) | Institute of Medical Science, | cluster/software.htm |
| | University of Tokyo | |
| JavaTreeView (v.1.1.6r4) | Stanford University School of | //jtreeview.sourceforge.net/ |
| | Medicine | |
| “edgeR” (v.3.28.0) | R | //bioconductor.org/packages/release/ |
| | | bioc/html/edgeR.html |
| “ggplot2” (v.3.2.1) | R | //cran.r-project.org/web/packages/ |
| | | ggplot2/index.html |
| GSEA (v.4.0.3). | Broad Institute | www.gsea-msigdb.org/gsea/index.jsp |
| (gene set enrichment analysis) | | |

### EXAMPLE 1

In this example, iPSC lines from healthy donor T cells subpopulation—naïve and memory T cells (T_{n/mem}) were generated by an integration-free method using iPSC reprograming episomal vectors (as described above and in, for example, Okita, K., et al., An efficient nonviral method to generate integration-free human-induced pluripotent stem cells from cord blood and peripheral blood cells. Stem Cells, 2013. 31(3): p. 458-66). The resultant iPS cells were transduced with clinical grade lentivirus to express CD19-specific CARs (CD19CAR) or other CAR. Single cell was sorted, colonized and screened to generate a homogeneous CAR+ iPSC cell bank.

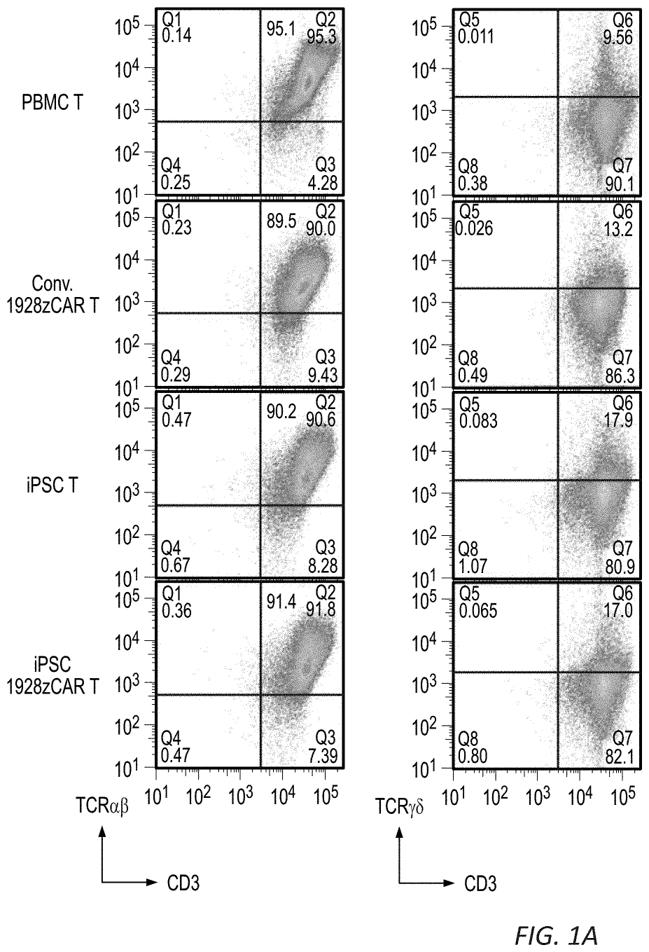

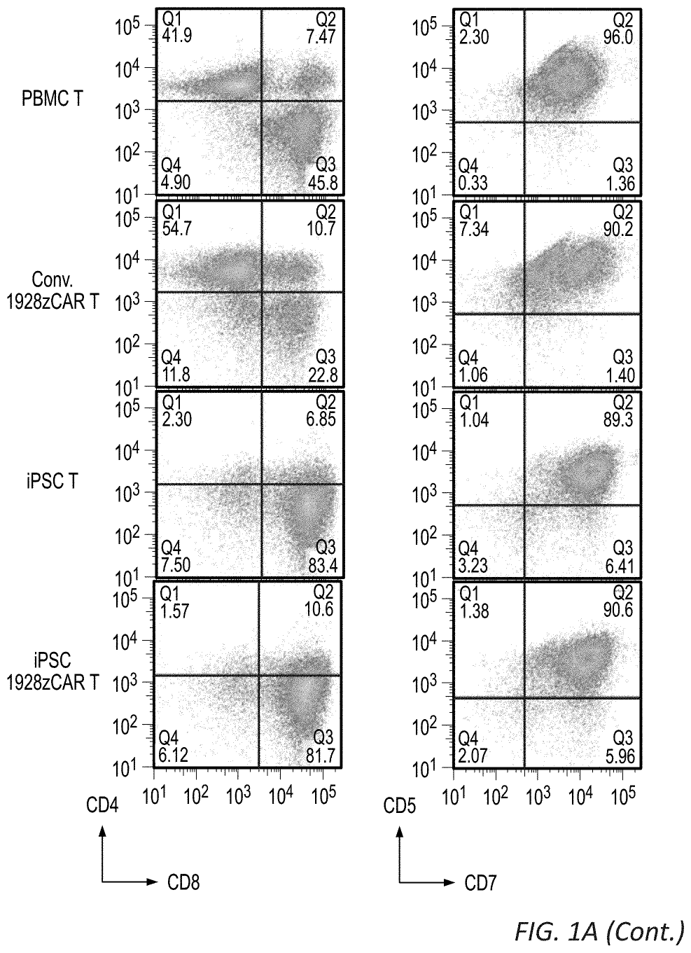

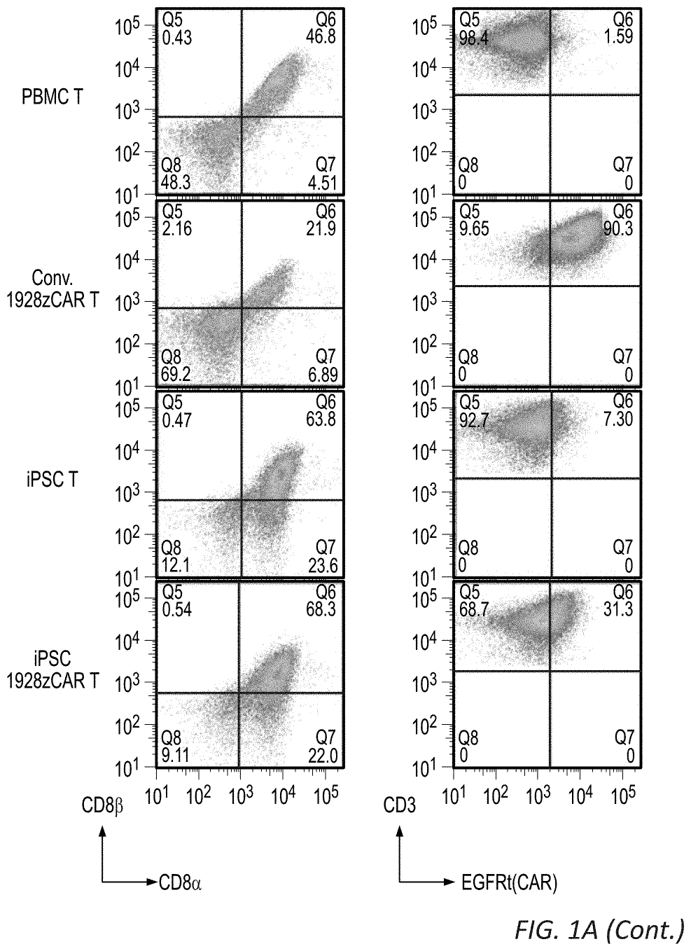

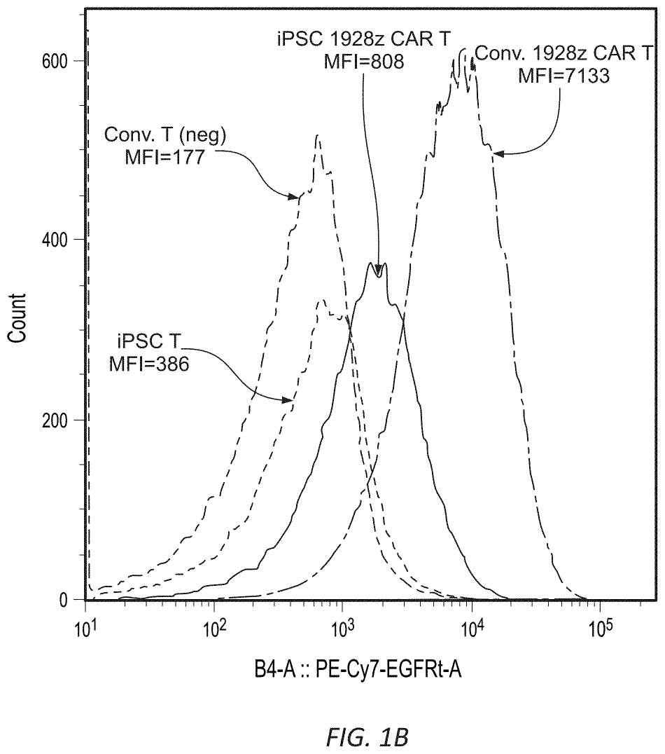

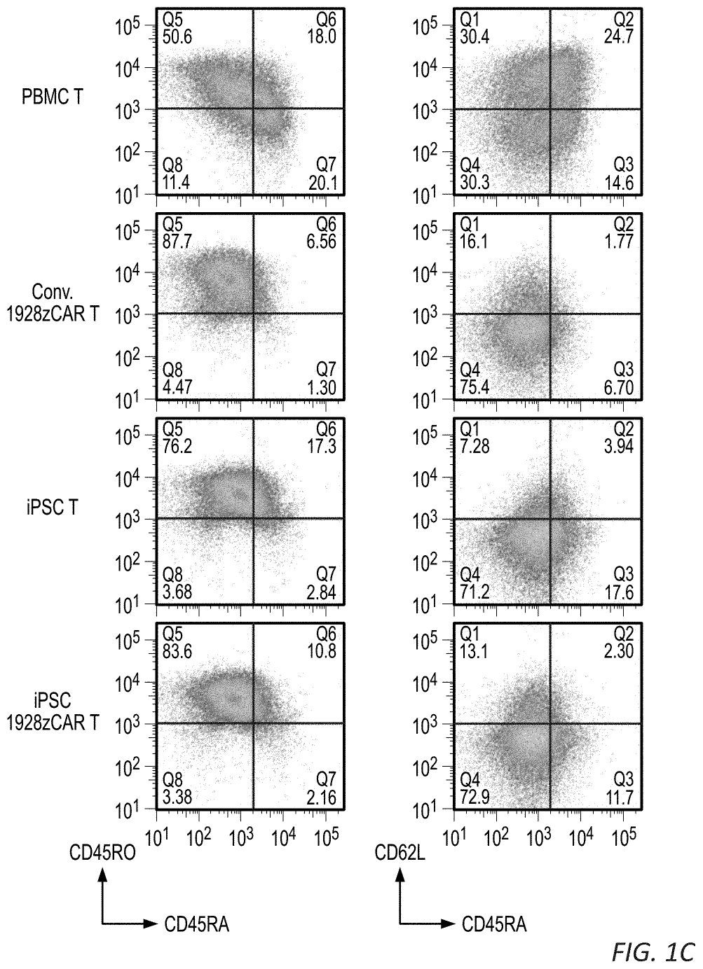

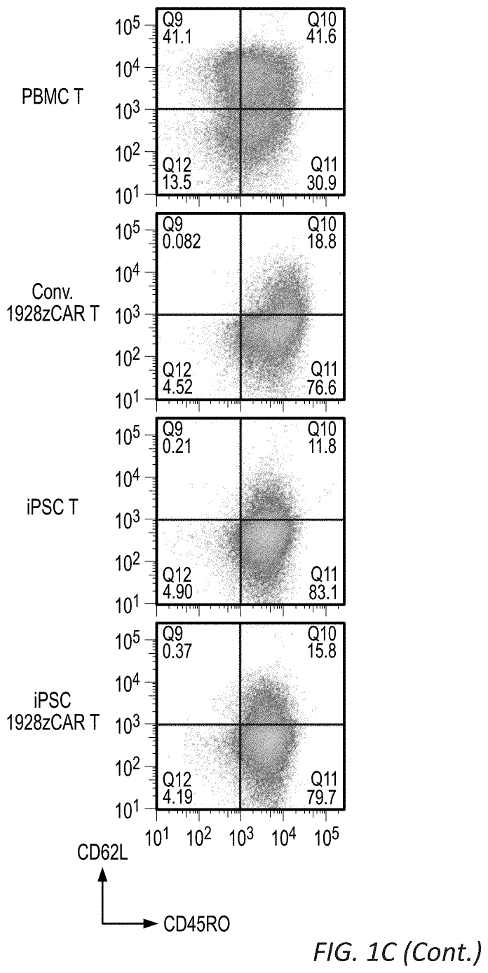

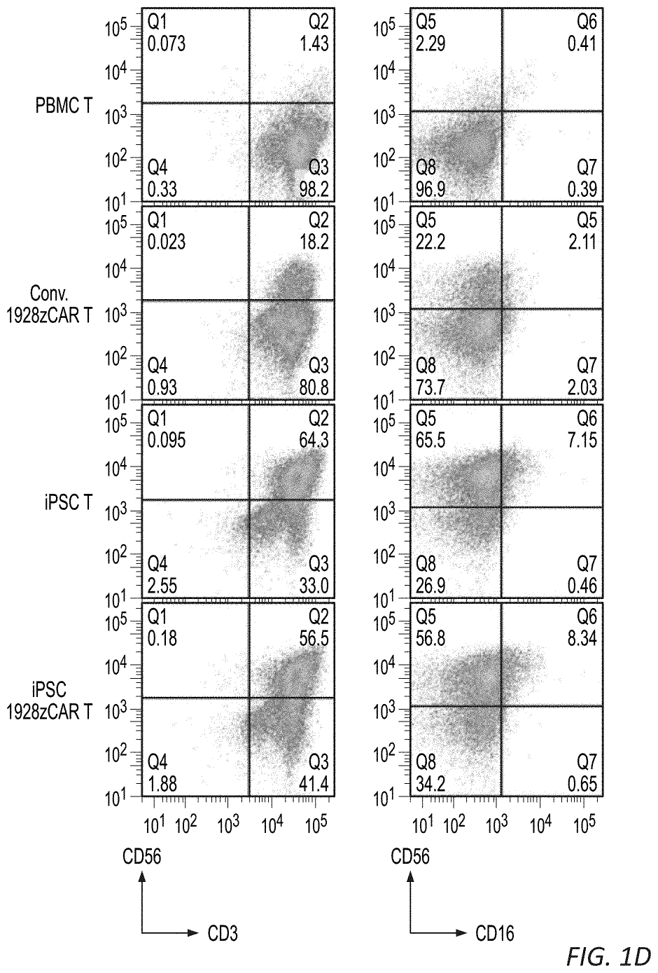

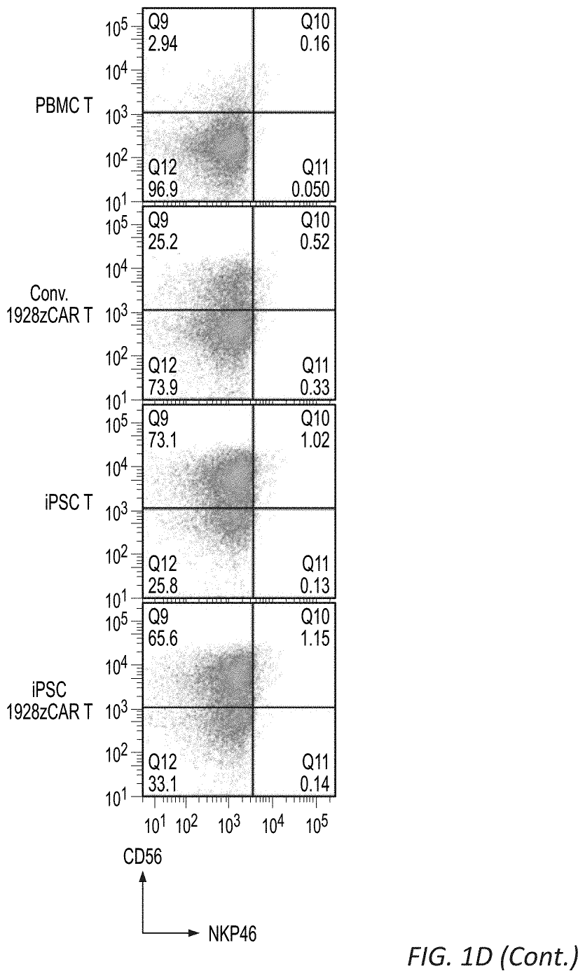

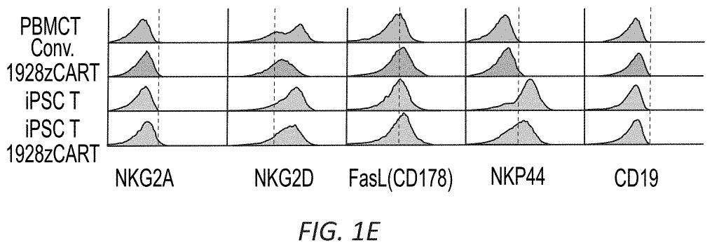

By using EMO-ATO culture system, as described above as Protocol 1A, T_{n/mem} iPSC-derived CAR T cells were successfully generated. The produced iPSC CD19CAR T cells have a conventional T cell surface marker phenotype with CD3+CD5+CD7+TCRαβ+CD8+ and CD3+CD5+CD7+TCRαβ+CD4+ (FIG. 1A). The expanded cells were composed of classical CD62L+CD45RA+stem memory T cells, CD62L-CD45RA-CD45RO+ effector memory T cells and CD62L-CD45RA+effector T cells (FIG. 1C). The CAR expression level in T_{n/mem} iPSC 1928zCAR T cells was lower than CAR T cells generated from PBMC cells from same donor. Notably, these cells did not express NK cell specific marker NKP46 and CD16 (FIG. 1D), which is different from mono-layer co-culture generated T cells (as described in Themeli, M., et al., Generation of tumor-targeted human T lymphocytes from induced pluripotent stem cells for cancer therapy. Nat Biotechnol, 2013. 31(10): p. 928-33). The iPSC CD19CAR T cells expressed similar levels of pan cytotoxic receptor molecular NKG2D, higher levels of NKP44, and negatively express NKG2, and B cell lineage marker CD19 (FIG. 1E).

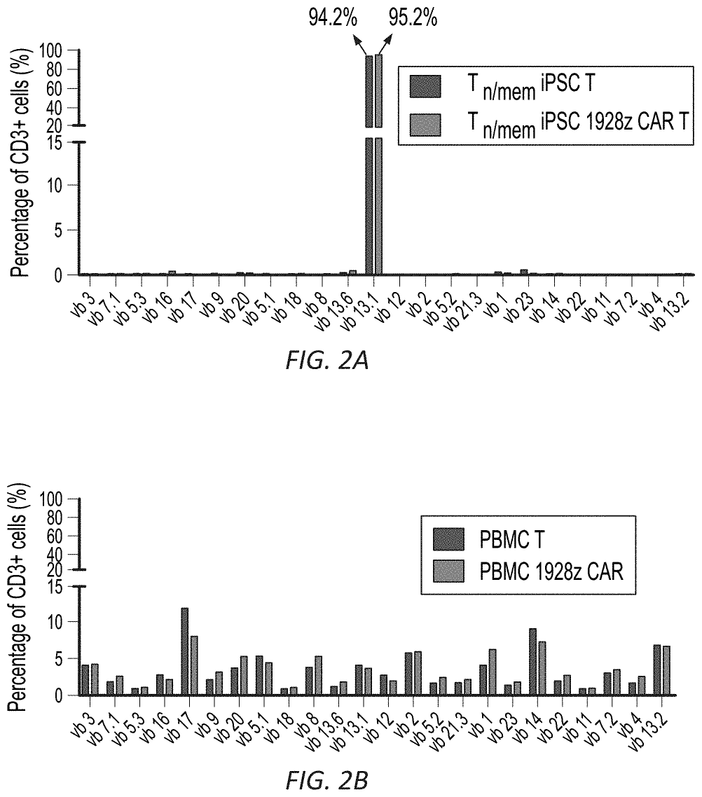

A flow cytometry based TCR Vβ repertoire expression assay demonstrated that the T_{n/mem} iPSC 1928zCAR T cells showed one type of TCR repertoire exclusively (FIGS. 2A-2B). This phenotype is similar to TCR transgenic expression induced allelic expression effect (Brady, B. L., N. C. Steinel, and C. H. Bassing, Antigen receptor allelic exclusion: an update and reappraisal. J Immunol, 2010. 185(7): p. 3801-8), which hold potential application to generate pure antigen specific T cells to reduce unwanted graft versus host effect (GvH). Thus, in some embodiments, the CAR T cells disclosed herein reduce at least one symptom associated with GvH.

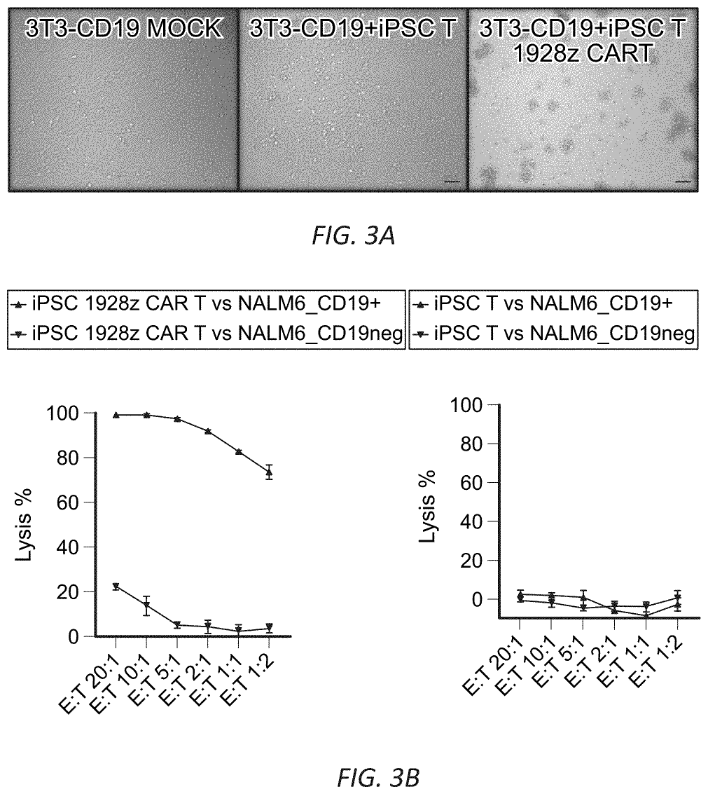

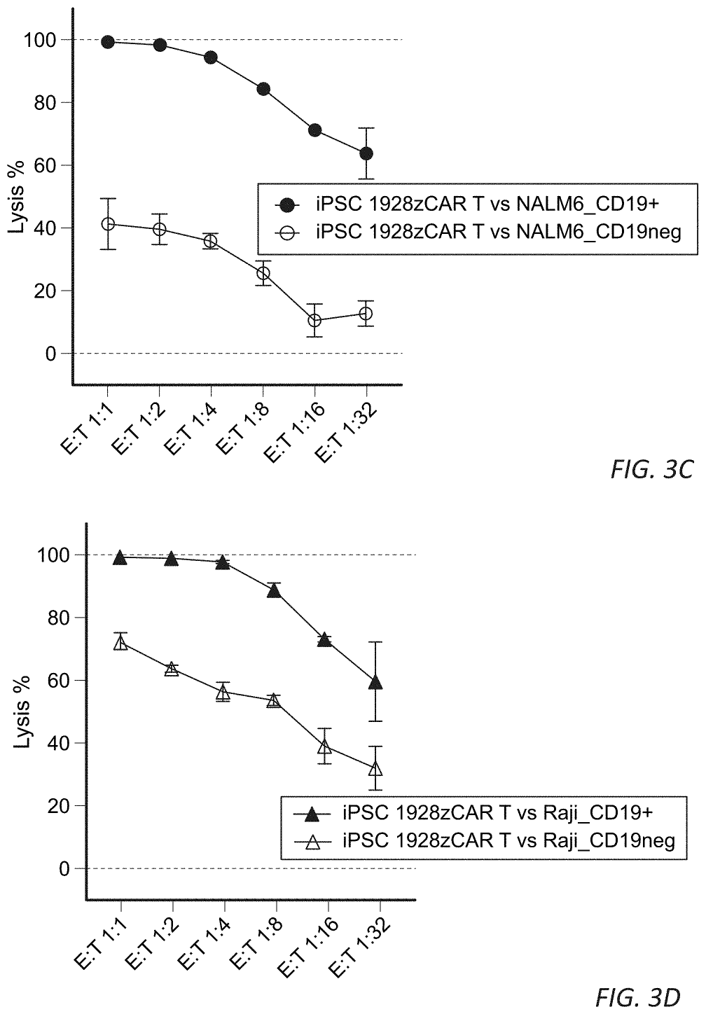

The T_{n/mem} iPSC 1928zCAR T cells expanded robustly within two weeks (˜100 fold), and showed potent antigen-specific cytotoxicity against CD19+ target cells such as CD19+ 3T3 cells, parental tumor cells NALM6, and Raji as compared to their CD19 knockout control cells (FIGS. 3A-3C). The in vitro cytotoxicity potency of iPSC-derived CD19CAR T cells was superior to conventional PBMC-derived CAR T cells generated from the same donor (FIG. 3D).

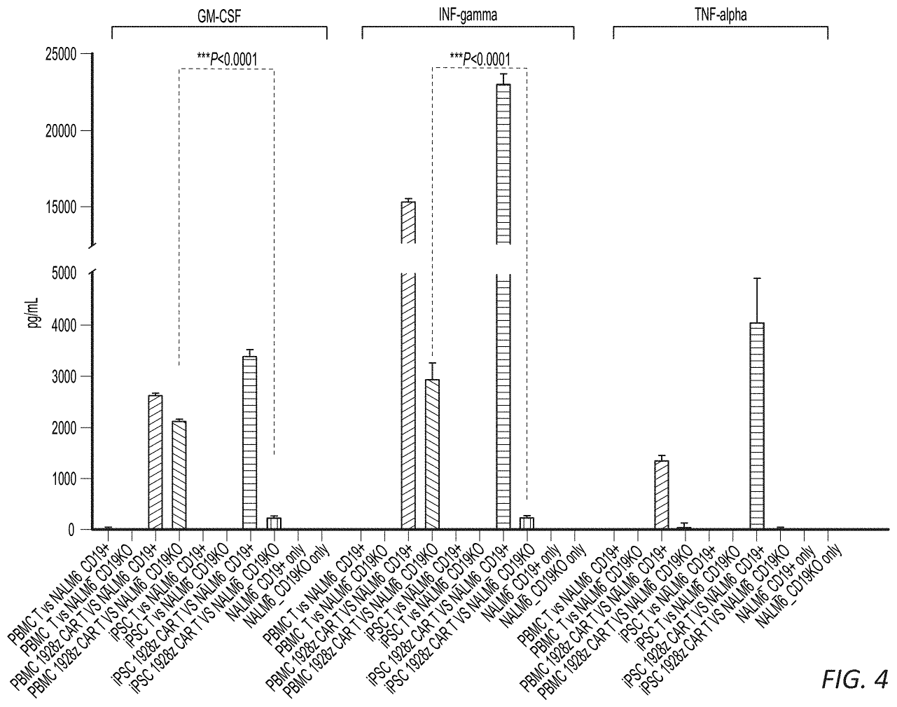

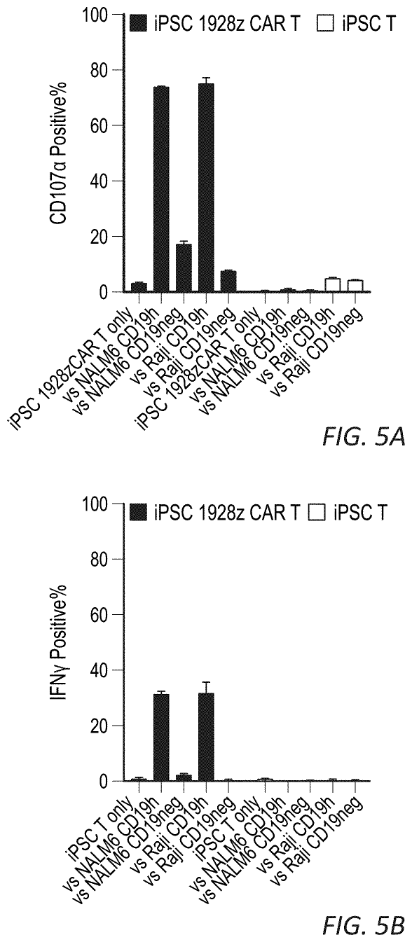

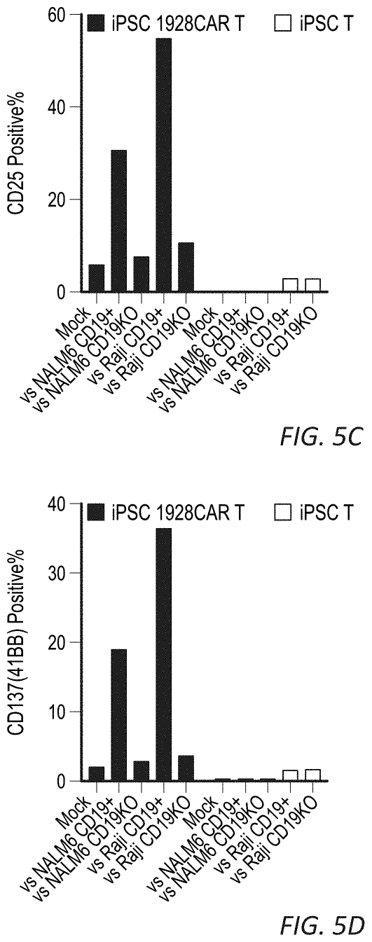

The iPSC-derived CD19CAR T cells also demonstrated efficient degranulation and activation phenotype (FIG. 4). The cytokine profile upon CD19+ positive cancer cell challenge was examined. The T_{n/mem} iPSC 1928zCAR T cells can potently secrete Th1 cytokines IFNγ and TNFα. The T_{n/mem} iPSC 1928zCAR T cells secrete lower level of GMC-SF, IFNγ and TNFα in static state without CD19 antigen challenge. It would be expected to have less cytokine release syndrome in vivo.

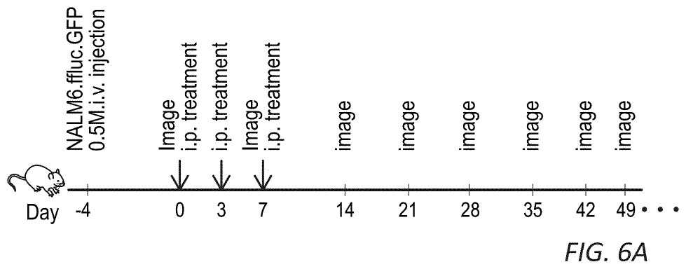

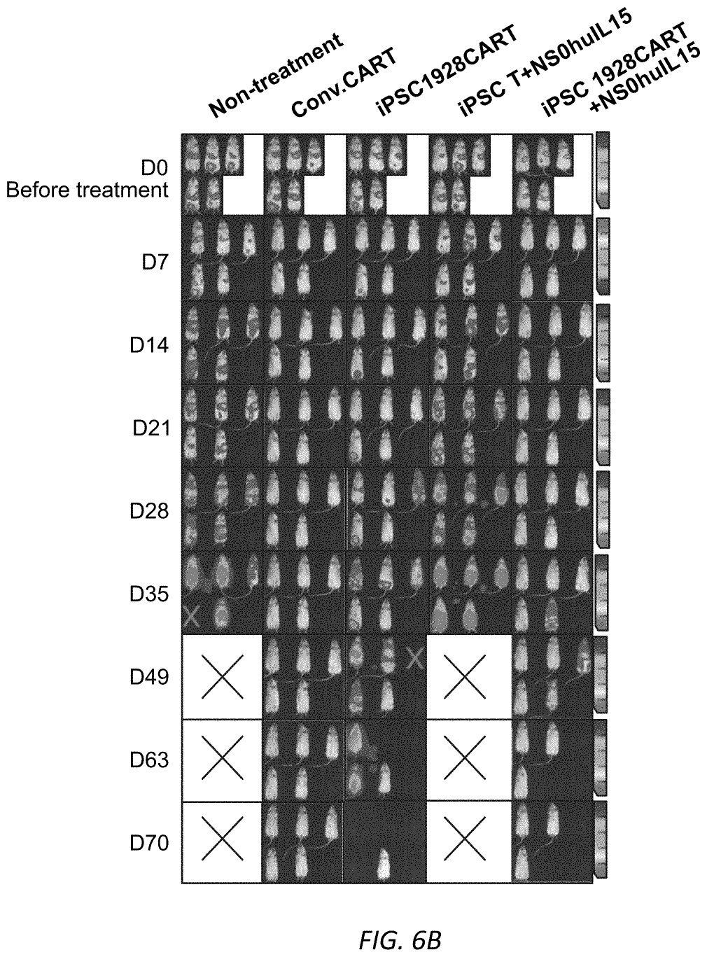

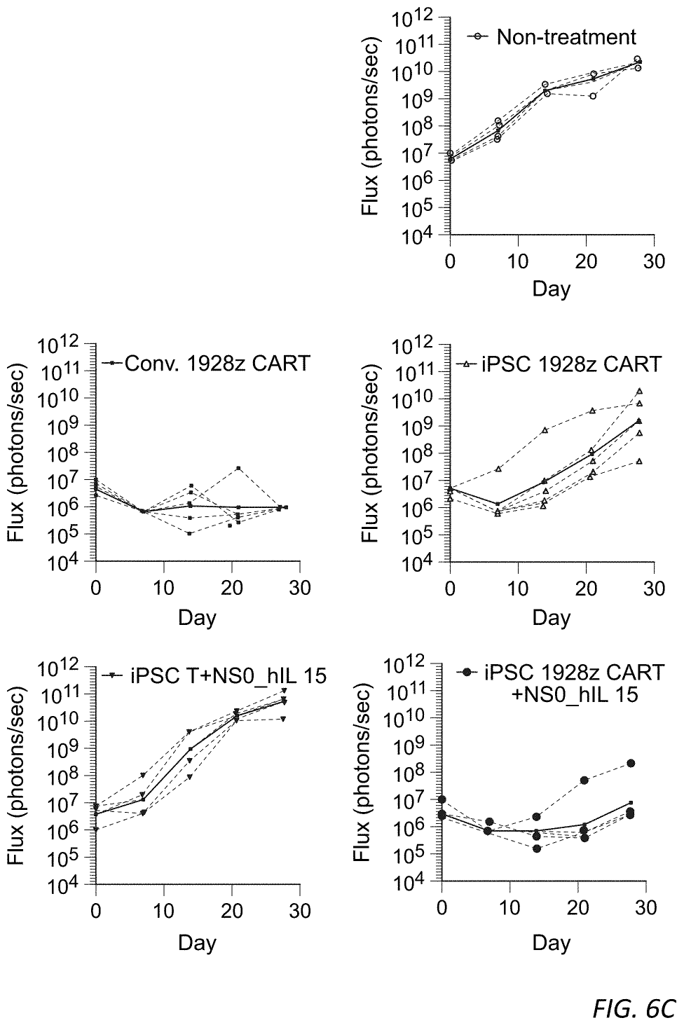

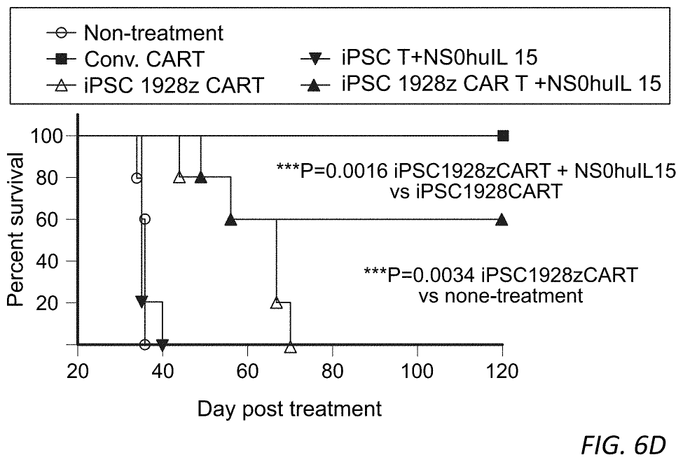

The anti-tumor activity of T_{n/mem} iPSC 1928zCAR T cells in vivo was examined in a NSG mouse model engrafted with NALM6 cells. T_{n/mem} iPSC 1928zCAR T cells significantly eliminated the engrafted tumor cells and improved the mice survival. The combination of T_{n/mem} iPSC 1928zCAR T cells and IL15 secretory nurse cells IL15_NS0 further improved the therapeutic effects (FIGS. 6A-6D).

### EXAMPLE 2

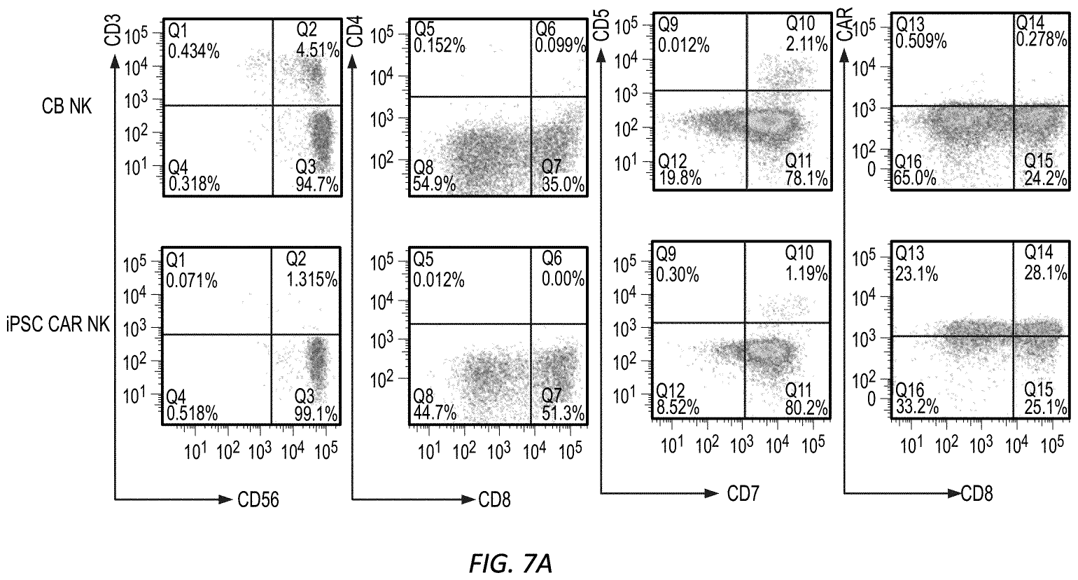

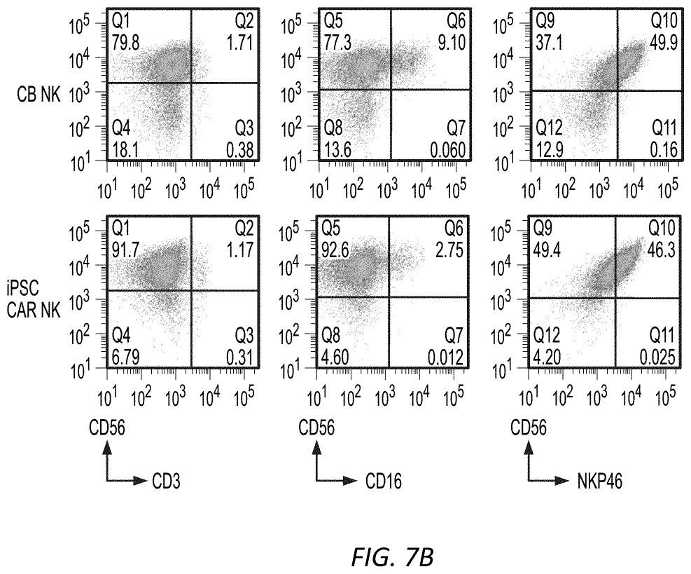

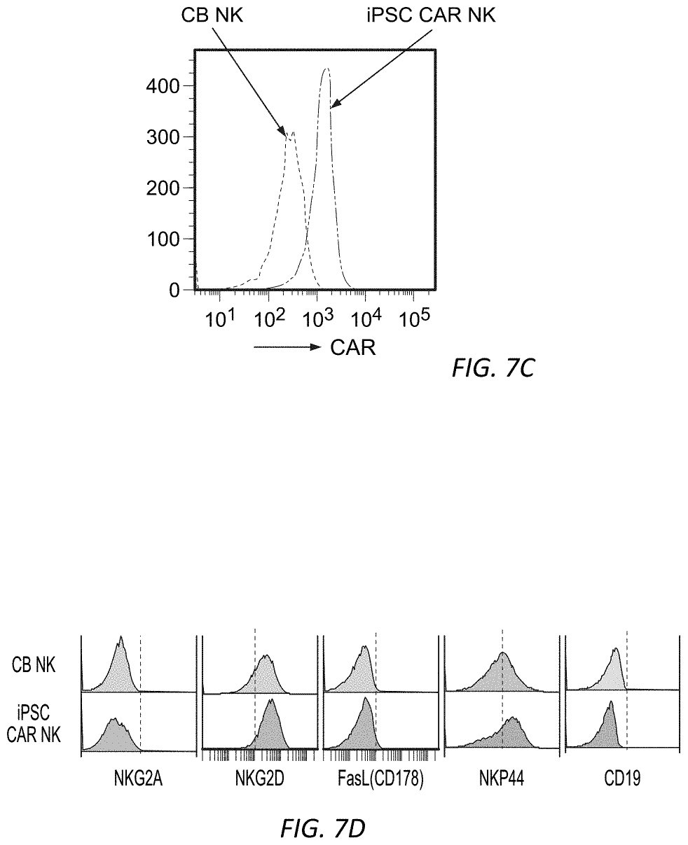

Tn/mem iPSC HSPC-derived CAR NK cells were generated as described above, for example, as in protocol 2B. The generated CAR NK cells demonstrated typical NK marker profile of CD3-CD56+ NKP46+ (FIGS. 7A-7D). They also expressed NKG2D, NKP44 and low level of CAR.

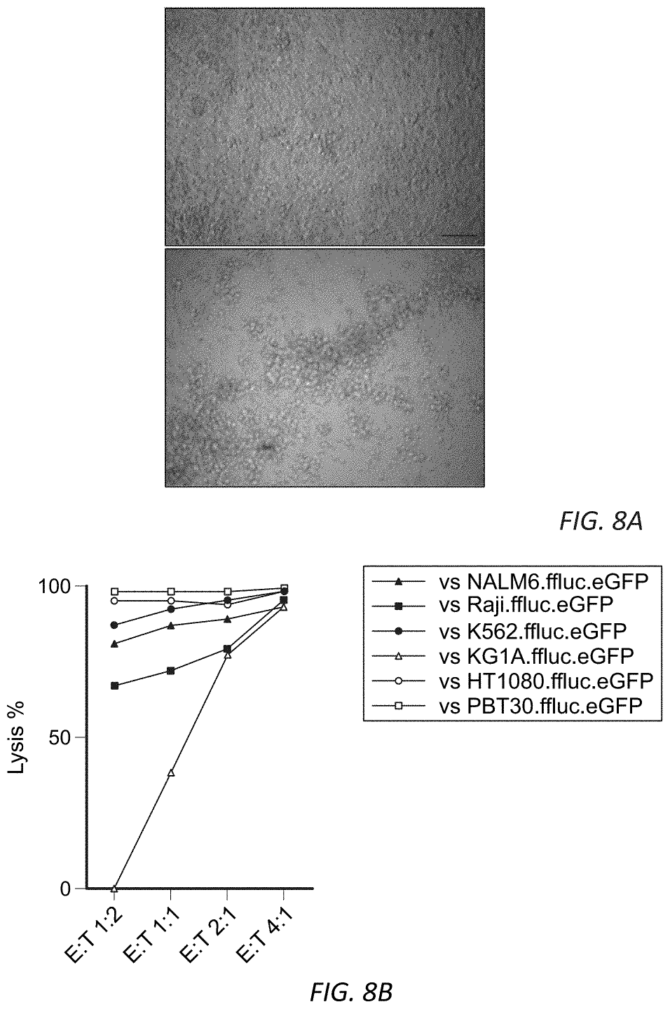

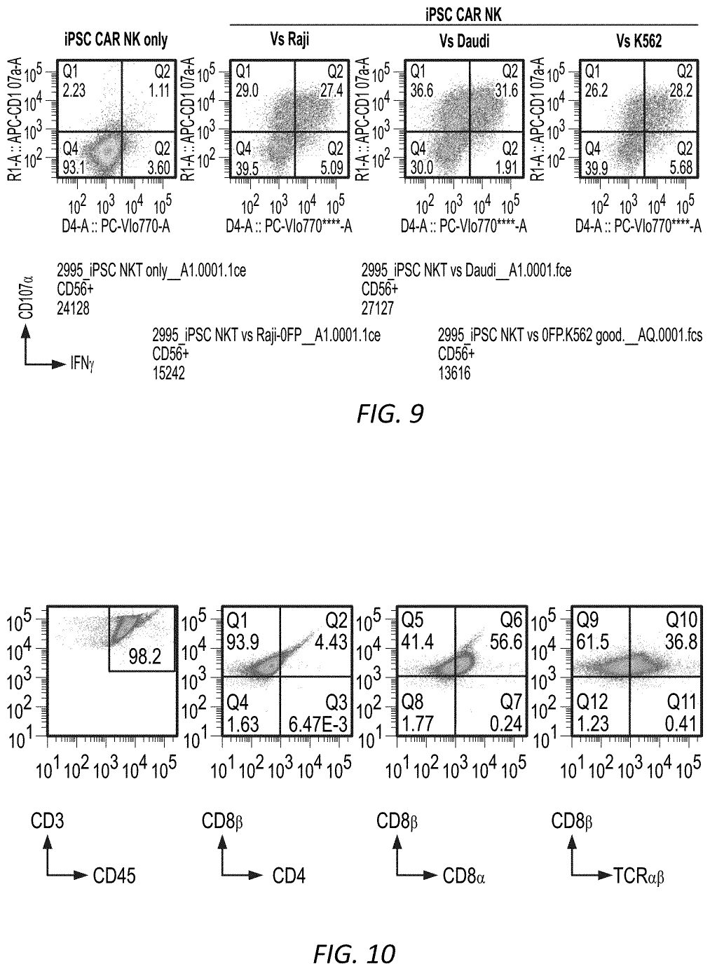

Tn/mem iPSC HSPC-derived 1928zCAR NK cells were functional and demonstrated potent cytotoxicity against a panel of tumor cell lines in antigen-dependent and antigen-independent manners (FIGS. 8A-8B). They also showed potent degranulation activity when co-cultured with tumor cells (FIG. 9).

### EXAMPLE 3

The iPSC 1928zCAR T cells were also generated by nanofiber matrix based culture as described above, for example, as in Protocol 3A. The generated 1928zCAR T cells also demonstrated conventional T cells phenotype with CD3+CD8αβ+ or CD3+CD4+ (FIG. 10).

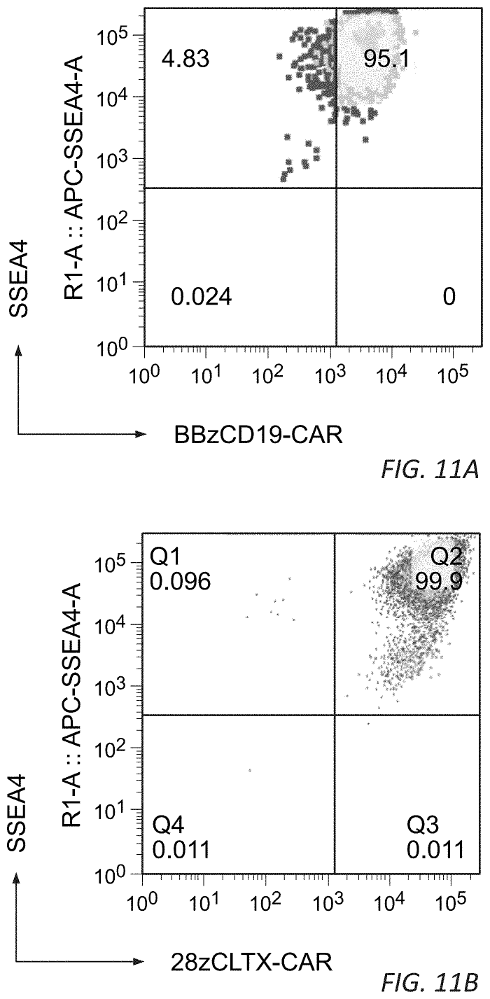

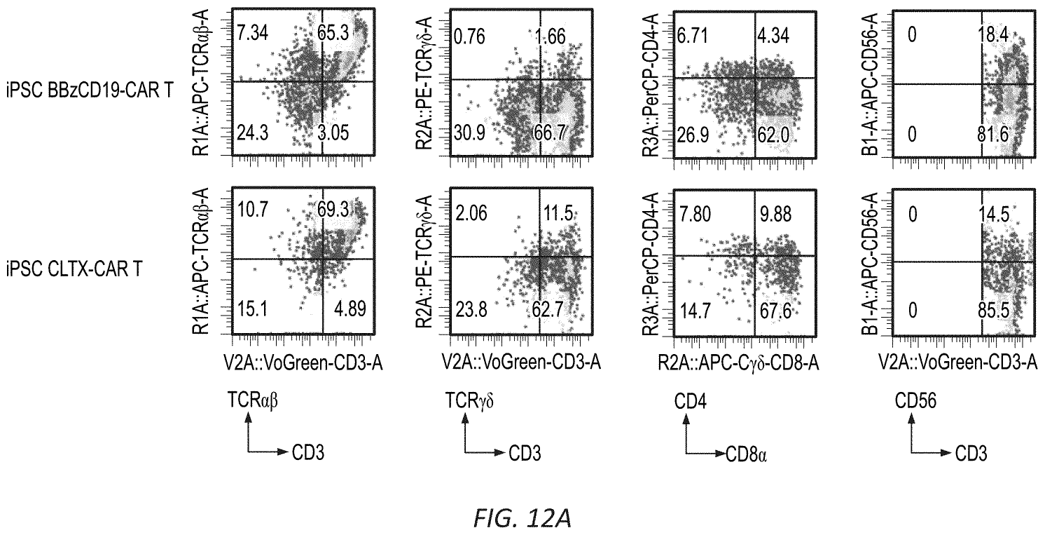

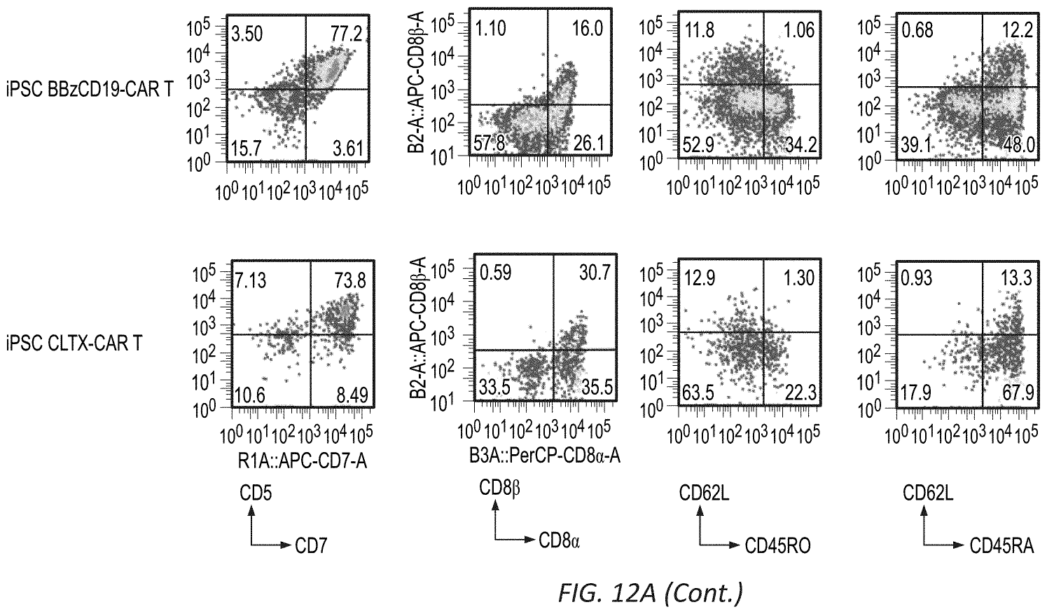

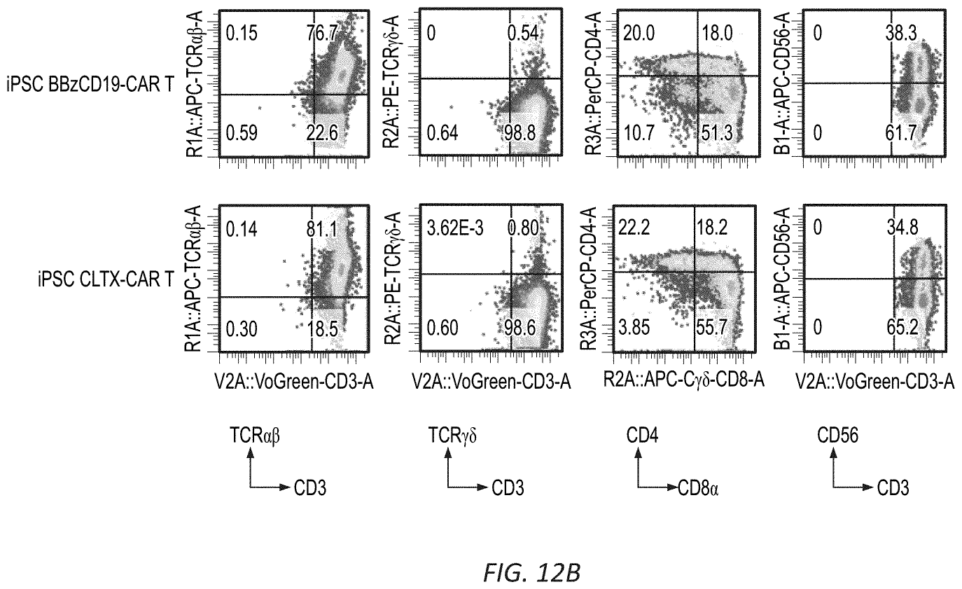

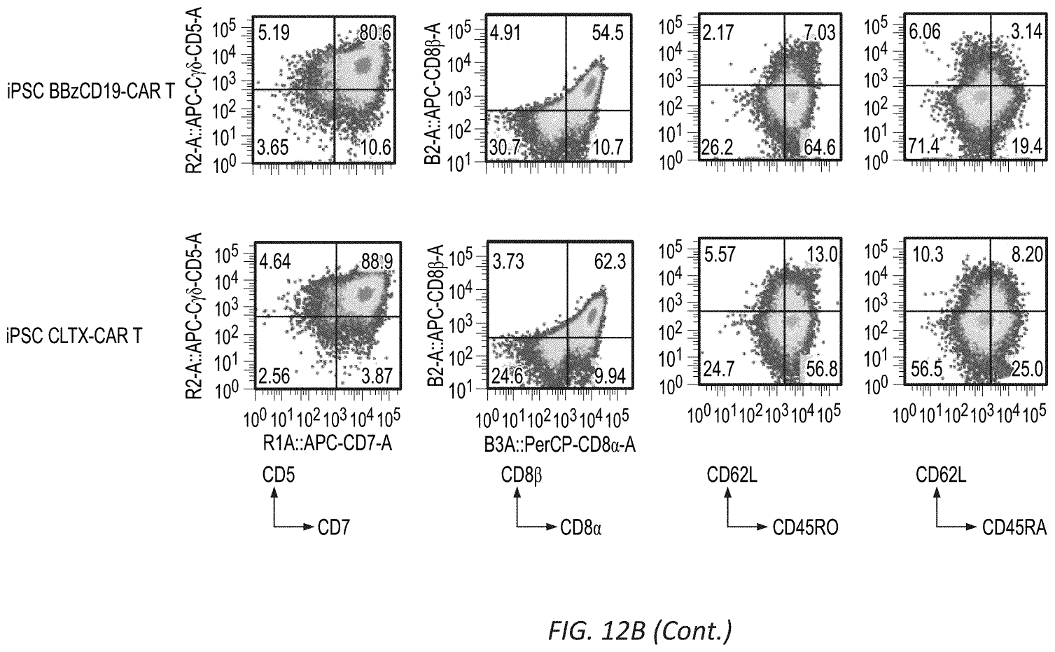

As seen in FIGS. 11A-11B, the colonized iPSC lines express BBzCD19-CAR and 28zCLTX-CAR and do not have high expression of stage-specific embryonic antigen-4 (SSEA-4). The cell surface expression of the iPSC derived BBzCD19-CAR T and 28zCLTX-CAR T cells is shown in FIGS. 12A-12B (12A: iPSC CAR T phenotype at week 7 without REM expansion; B: iPSC CAR T phenotype after REM expansion).

### EXAMPLE 4

This example also shows use of iPSC differentiation for generation of CAR T cells with canonical T cell phenotype and CAR T function. The publications referenced in this example are listed at the end of the example.

Summary

This example shows that unlimited generation of chimeric antigen receptor (CAR) T cells from induced pluripotent stem cells (iPSCs) can be used for the development of ‘off-the-shelf’ CAR T cell immunotherapy. Approaches that enable efficient directional differentiation of iPSCs into canonical αβ T cell lineages, along with maintenance of CAR expression and functionality, however, are challenging. Described below is q continuous 3D-organoid system facilitates the generation of T cells from CAR-engineered iPSCs and confer products with conventional CAR T cell characteristics. The iPSCs were reprogrammed from an enriched CD62L+ naïve and memory subsets (Tn/mem) followed by CAR transduction, single cell sorting, and colonization. Induction of T cell directional differentiation via 3D-organoid culture was evident in that the resulting CD19-CAR T cells (iPSC CD19-CAR T cells) were predominantly CD3/CD5/CD7/TCRαβ/CD8αβ-positive and TCRγδ-negative. While iPSC CD19CAR T cells exhibited lower CAR expression levels due to hyper-methylation of the EF1α promoter as compared to conventionally derived CAR T cells, they exhibited better antigen specificity in cytokine release and more robust TCR/CAR signaling. Expanded iPSC CD19-CAR T cells showed comparable antigen-specific activation, degranulation, cytotoxicity and cytokine secretion compared to conventional CD19-CAR T cells generated from donor matched PBMC, and they maintained homogenous expression of the TCR derived from the initial clone. iPSC CD19-CAR T cells also exhibited antitumor activity in vivo, prolonging survival of CD19+ human tumor xenografted mice. In summary, these methodologies generate highly functional conventional CAR T cells from iPSCs to support the development of ‘off-the-shelf’ manufacturing strategies.

Results

Generation of iPSC-Derived CAR T Cells with a Conventional T Cell Phenotype

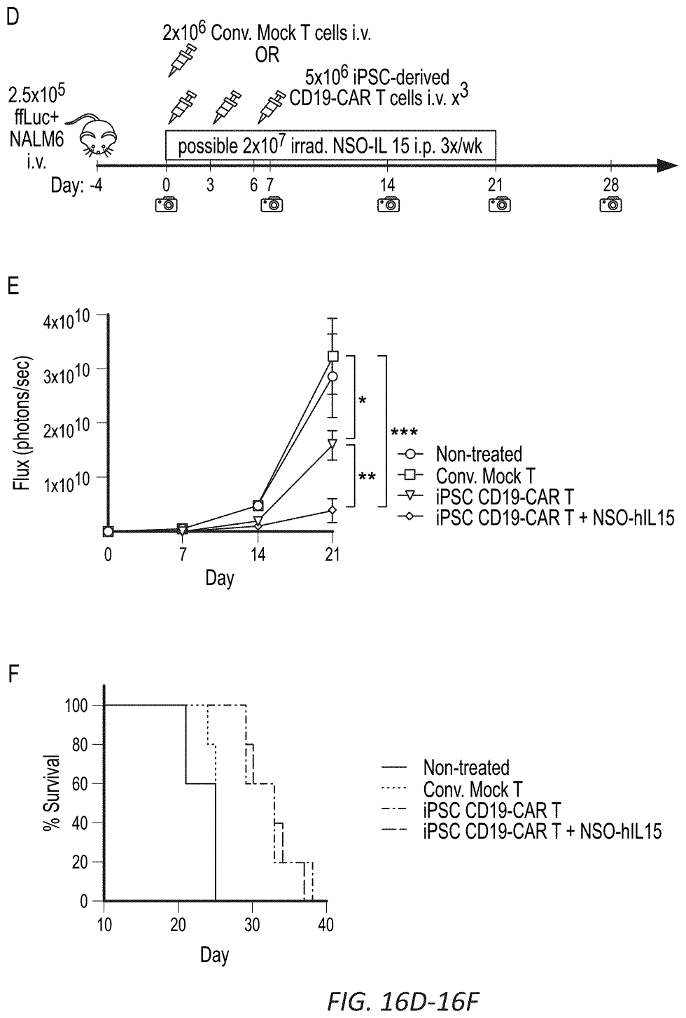

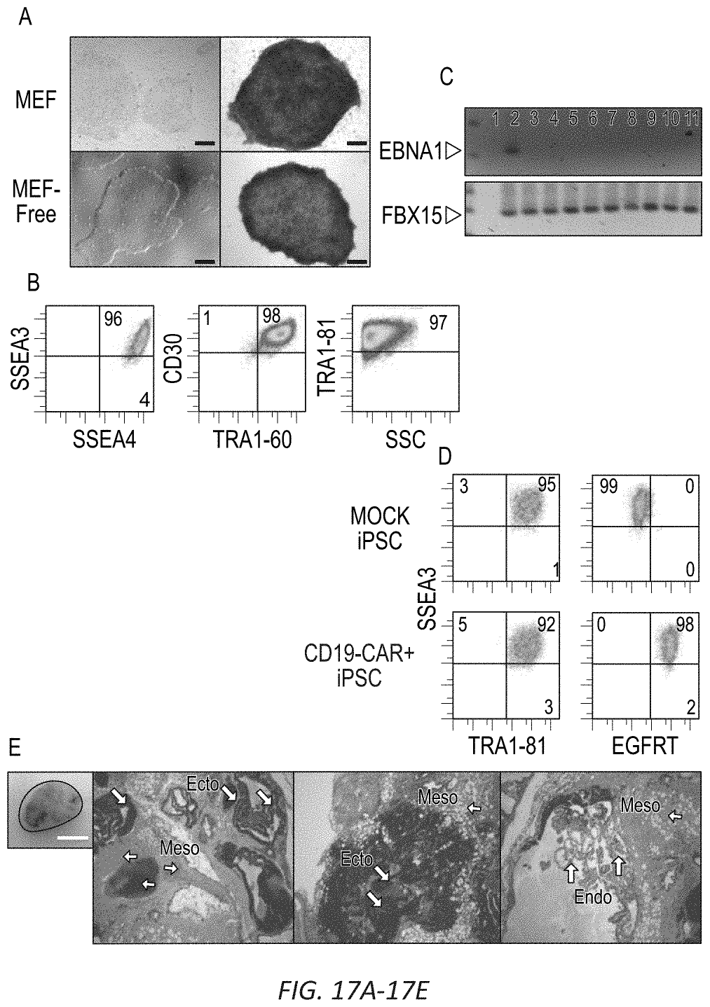

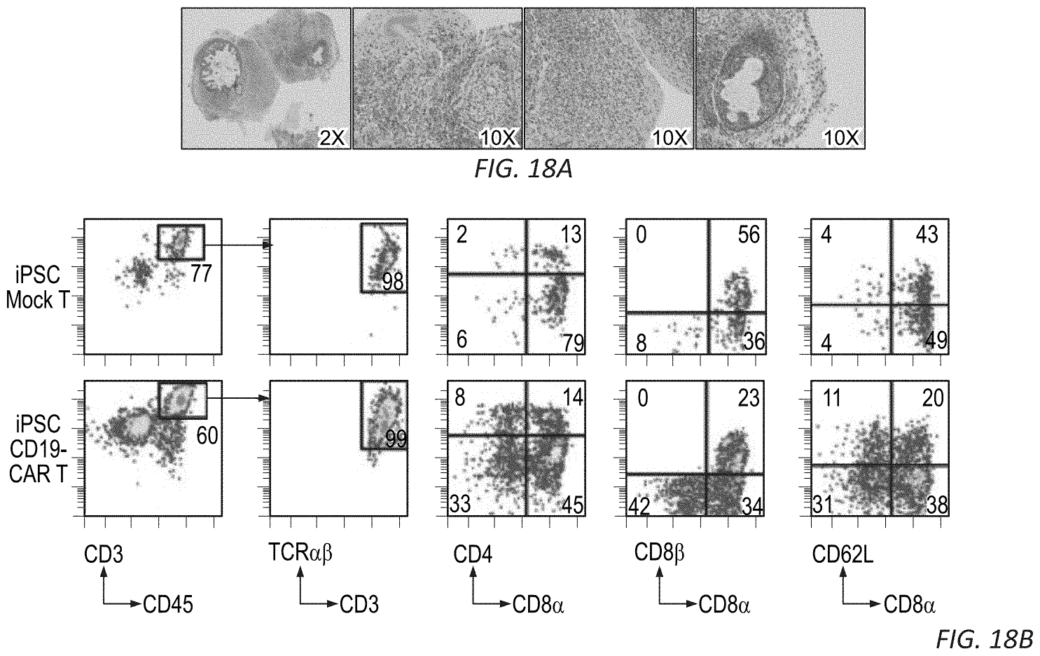

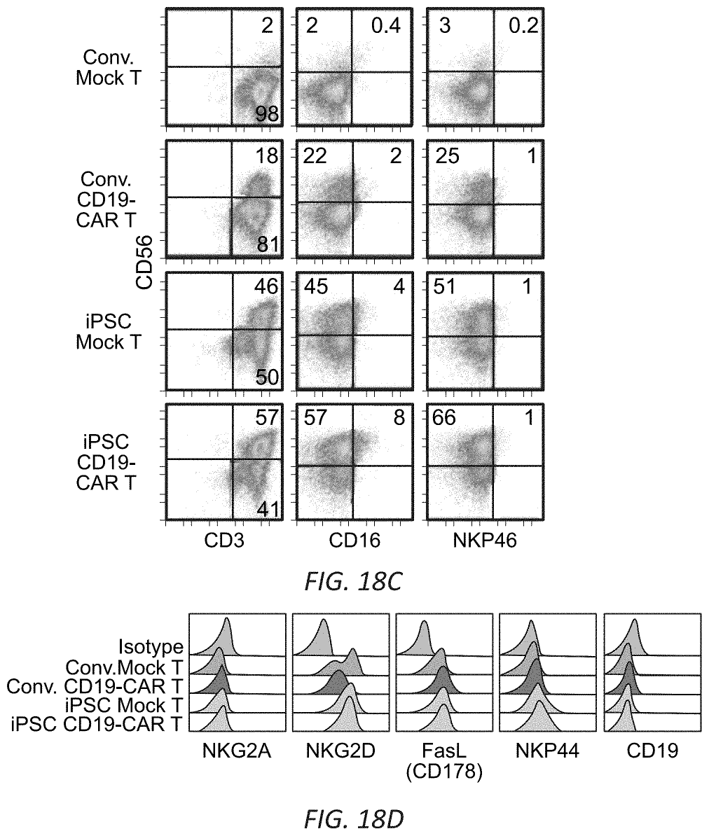

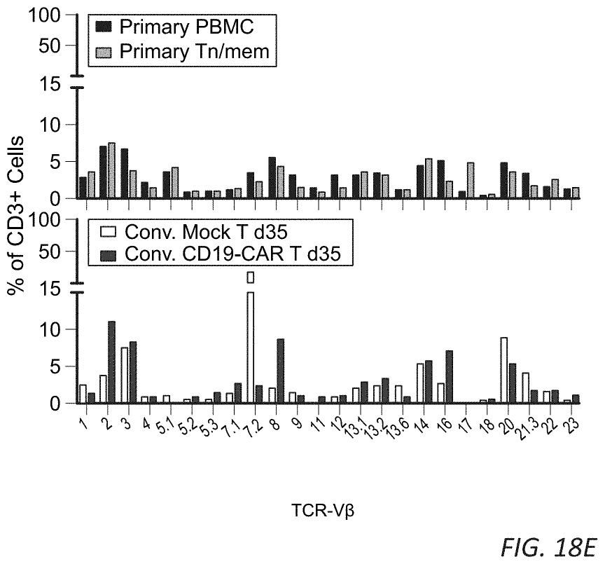

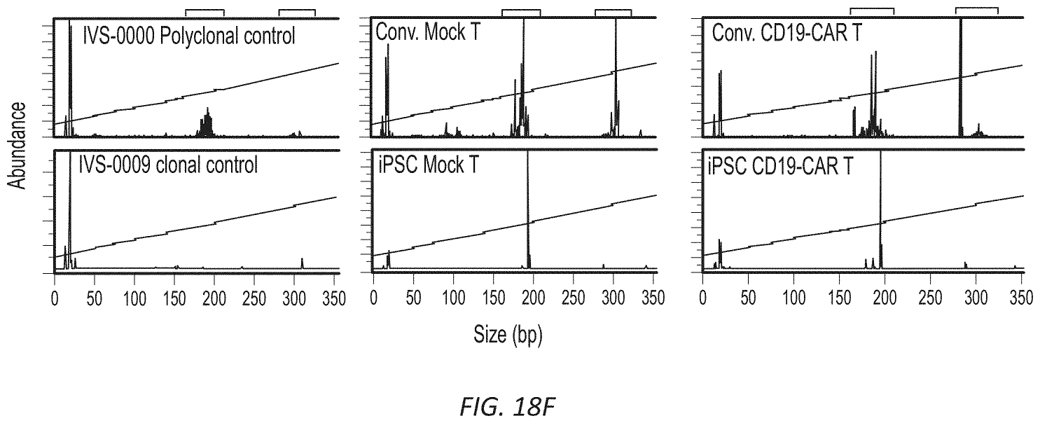

The iPSC clones were derived from primary CD62L+ naïve and memory T cells (Tn/mem), a T cell population that has been proposed to have superior persistence and improve clinical outcomes in CAR T cell therapy (McLellan and Ali Hosseini Rad, 2019; Morgan and Schambach, 2018; Popplewell et al., 2018; Samer K. Khaled, 2018; Zah et al., 2020). The Tn/mem cells enriched from the peripheral blood of healthy human donors were transduced and reprogrammed by episomal plasmids encoding KLF4, SoX2, OCT-4, C-MYC and LIN28, along with P53 shRNA (Okita et al., 2013), and multiple integration-free iPSC clones were screened and characterized (FIGS. 17A-17E). iPSC clone pluripotency was confirmed by alkaline phosphatase staining and examination of stem cell markers SSEA3, SSEA4, TRA1-60, TRA1-81 and CD30 (FIGS. 17A-17B), with EBNA PCR demonstrating that the iPSC clones were integration free (FIG. 17C) Qualified clones were transduced with clinical grade lentivirus encoding a CD19-targeting CAR (Popplewell et al., 2018; Samer K. Khaled, 2018) and CAR+ cells were single cell sorted by flow cytometry, colonized, expanded, and banked. Both mock-transduced and CAR-expressing clones maintained stem cell marker expression (FIG. 17D). The parental iPSC and CD19-CAR+ iPSC clones were further tested by teratoma formation assay to confirm their pluripotency potential to generate ectoderm, endoderm and mesoderm germ layers (FIG. 17E). To direct the differentiation of CD19-CAR expressing iPSC into CD19-CAR expressing T cells, the modified embryonic and induced pluripotent stem cells Artificial Thymic Organoid (PSC-ATO) system of Montel-Hagen et al (Montel-Hagen et al., 2019), was modified. First, the CD19-CAR+ iPSCs were cultured in feeder-free conditions for the first three days to induce mesodermal differentiation (FIG. 13A). CD56+CD326− iPSC mesodermal progenitor cells (iMP) were then enriched by magnetic selection of CD56+cells, and went through iPSC mesodermal organoid culture (iMO) with MS5-hDLL4 feeder cells to differentiate into hematopoietic progenitors (14 days), followed by T cell commitment and differentiation (additional 5-7 weeks) (FIG. 13A). In situ staining and imaging of mature organoid cultures demonstrated a heterogeneous tissue-like architecture with CD3⁺ T cells and GFP⁺ MS5-DLL4 feeder cells (FIG. 13B, FIG. 13C, and FIG. 18A). The cell yield from CD19-CAR+ iMP was comparable to that from mock-transduced iMP (FIG. 13D), and PSC-ATO differentiated iPSC T cells, both mock-transduced or CD19-CAR+, can be efficiently expanded to clinically relevant numbers using a modified rapid expansion method (REM) (Wang et al., 2011b), with approximately 75-fold expansion in 2-weeks (FIG. 13E). The resulting expanded iPSC CD19-CAR T cells were then harvested for phenotypic characterization and expansion. The PSC-ATO-differentiated and expanded iPSC CD19-CAR T cells demonstrated a CD3/CD5/CD7/TCRαβ/CD8αβ-positive, NKG2A/NKP46/CD16/CD19-negative phenotype. As a benchmark for conventional CAR T cell phenotype and function we utilized PBMC-derived CD19-CAR T cells generated from the same donor using standard CD3/CD28 bead stimulation procedures, and demonstrate that iPSC CAR T cells are phenotypically similar to the CD8⁺ subpopulation of conventional CD19-CAR T cells (FIG. 13F, FIG. 13G, FIG. 18B, FIG. 18C, and FIG. 18D). Furthermore, similar to conventional CD19-CAR T cells, the iPSC CD19-CAR T cells are composed of populations in different stages of differentiation, including naïve or stem-cell-like T cells and memory T cells based on CD62L, CD45RA and CD45RO profiles (FIG. 13F-13G). They also express similar levels of FasL, but higher levels of CD56, NKG2D and NKP44 compared to conventional CD19-CAR T cells (FIG. 18C-18D). Interestingly, iPSC CD19-CAR T cells appear to express less CAR/transgene than conventional CD19-CAR T cells (FIG. 1H). TCR repertoire analysis by flow cytometry (FIG. 13I and FIG. 18E) and PCR of gDNA (FIG. 18F) demonstrate that the iPSC mock-transduced and CD19-CAR+ T cells preserve their clonal TCR, while the conventional T cells are highly polyclonal.

Transcriptional Profile of iPSC-Derived CAR T Cells

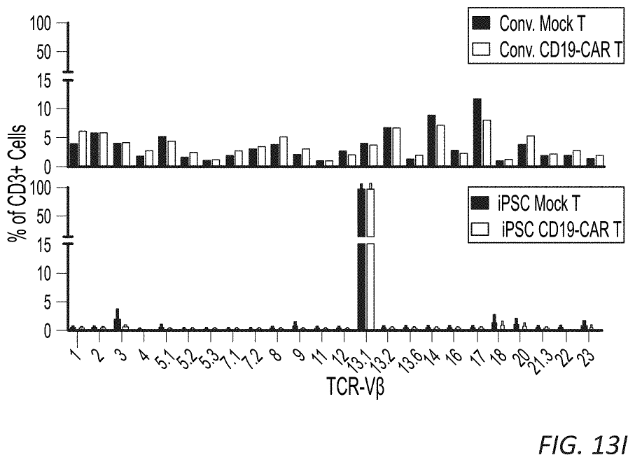

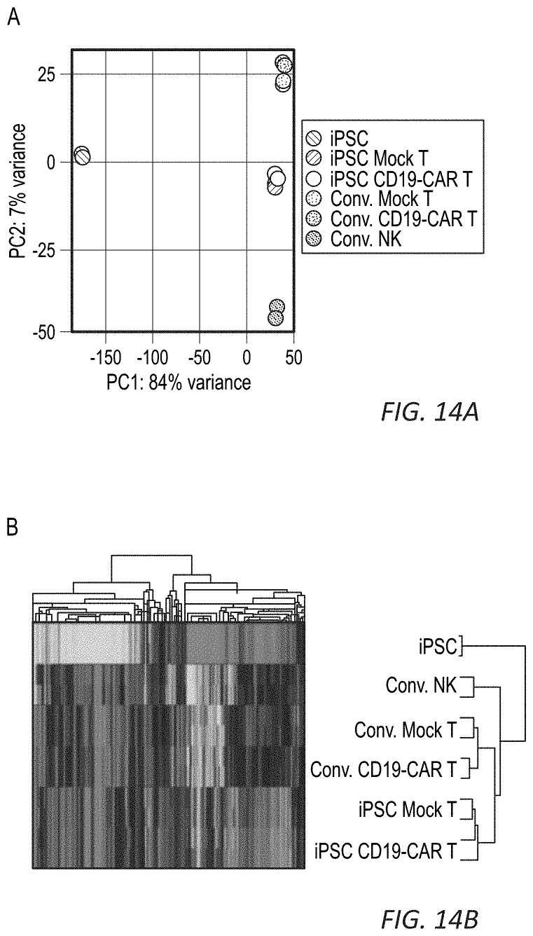

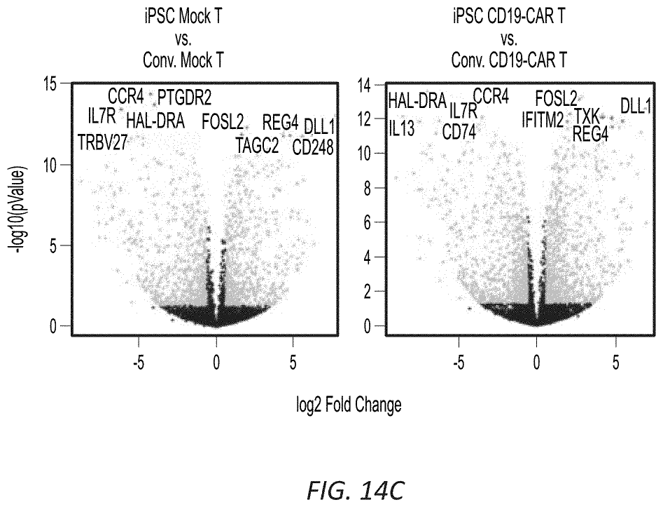

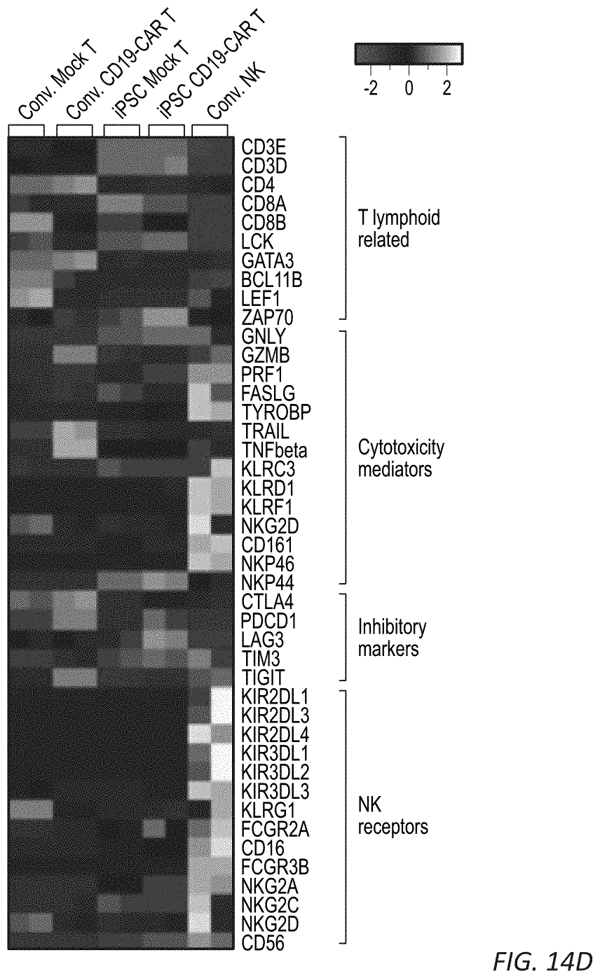

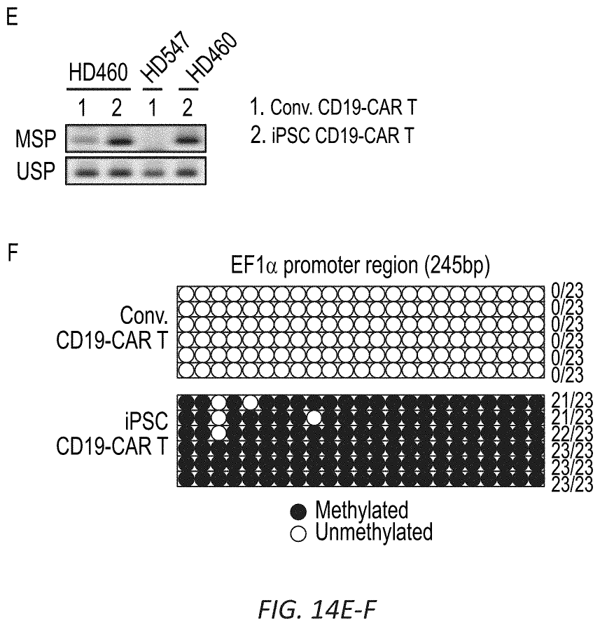

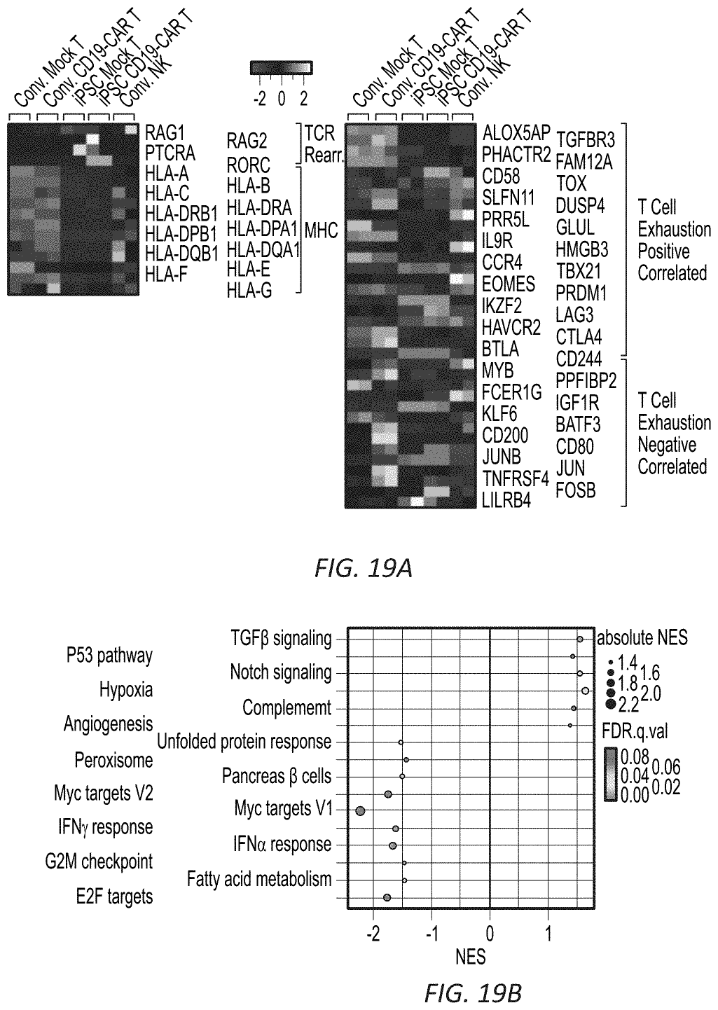

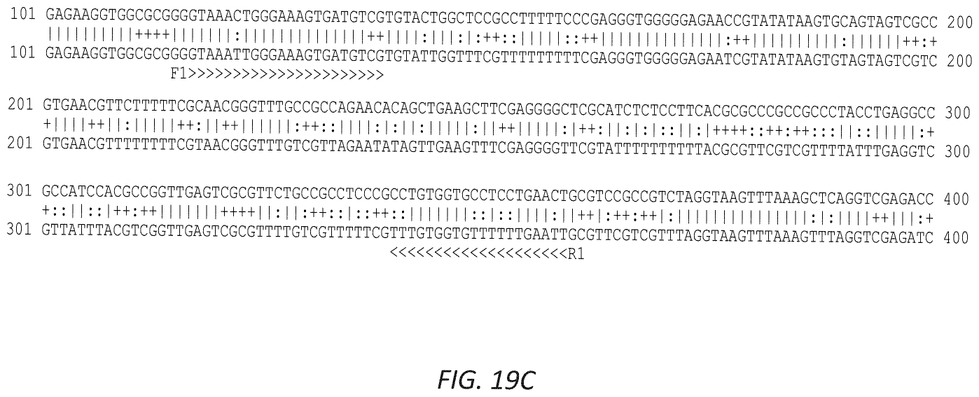

Bulk RNA deep sequencing analysis was used to explore differences between iPSC CD19-CAR T cells and conventional, PBMC-derived CD19-CAR T cells from the same donor. NK cells from the same donor were also used for comparison. Principle components analysis (PCA) showed that iPSC Mock T and iPSC CD19-CAR T cells displayed similar transcription profiles as conventional mock-transduced T cells, CD19-CAR T cells or NK cells derived from the same donor (PC score —7% variance), but were dramatically distinguished from iPSCs (PC score˜84% variance) (FIG. 14A). Hierarchical clustering of global transcriptional profiles showed that iPSC-derived T cells were more similar to conventionally derived T cells than to NK cells (FIG. 14B). Looking at the most significantly differentiated genes, it was observed that the iPSC CD19-CAR T cells expressed lower levels of IL-13, HLA-DR, IL7R, CCR4, and CD74, but higher levels of DLL1, FOSL2, TXK, REG4, and IFITM2 compared to the conventional CD19-CAR T cells (FIG. 14C). Evaluation of selected functional related gene sets revealed that iPSC CD19-CAR T cells expressed higher levels of T lymphocyte genes CD3E, CD3D, CD8, LCK and ZAP70, and lower levels of CD4, GATA3, BCL11B and LEF1 genes as compared to conventional CD19-CAR T cells (FIG. 14D). For cytotoxic mediator genes, iPSC CD19-CAR T cells express more GNLY and PRF1, but less GZMB compared to conventional CD19-CAR T cells. For T cell inhibitory genes, iPSC CD19-CAR T express less CTLA4, PD1, and TIGIT, but more LAG3 and TIM3 (FIG. 14D). iPSC CD19-CAR T cells do not express NK cell signature genes, which is similar to conventional CD19-CAR T cells (FIG. 14D). iPSC-derived T cells also demonstrated lower levels of MHC genes than conventional T cells and did not show biased gene signature towards exhaustion phenotype (FIG. 19A). Further, gene set enrichment analysis showed upregulated hypoxia and downregulated MYC target gene signatures in iPSC CD19-CAR T cells versus conventional CD19-CAR T cells (FIG. 19B), which may be related to the hypoxic microenvironment in 3D organoid culture and indicates unique metabolic signatures representing lower activation status in steady state comparing to conventional CAR T cells (Palazon et al., 2017; Pavlacky and Polak, 2020; Wang et al., 2011a).

As shown by flow cytometry that the iPSC CD19-CAR T cells expressed much lower levels of CAR transgene than conventional CAR T cells (FIG. 13H). However, the CAR expression levels in CAR transduced, colonized iPSCs was quite high and clearly distinguishable from mock-transduced iPSCs (FIG. 17D), indicating that subsequent CAR downregulation might be mediated by transcriptional or translational regulation during cell differentiation. Here, as in many lentivirus-based CAR T platforms (Porter et al., 2011; Programs, 2019), CAR transgene expression was driven by the EF1α promoter which contains many CpG islands (FIG. 19C). Thus, we investigated whether the CpG enriched EF1α promoter might be methylated during the cell differentiation of CD19-CAR expressing iPSC into CD19-CAR expressing T cells, and lead to transcriptional downregulation of the CAR. Examination of the methylation status by bisulfite specific PCR using bisulfite converted genomic DNA as template, showed that the EF1α promoter methylation status was significantly enhanced in iPSC CD19-CAR T cells when compared to conventional CD19-CAR T cells derived from the same donor (FIG. 14E). This hyper-methylation was confirmed upon further bisulfite sequencing analysis of a 245 bp region of the EF1α promoter containing 23 sites of CpG (FIG. 14F). These data suggest that EF1α promoter hyper-methylation occurs during T cell differentiation from iPSC, resulting in the downregulated CAR expression in iPSC CD19-CAR T cells. Taken together, iPSC CAR T cells have an RNA expression signature overall similarly to conventional CAR T cells while imply relatively less active status in steady state, which is accompanying with lower CAR expression level caused by transgene promoter hyper-methylation during differentiation.

Functional Analysis of iPSC-Derived CAR T Cells

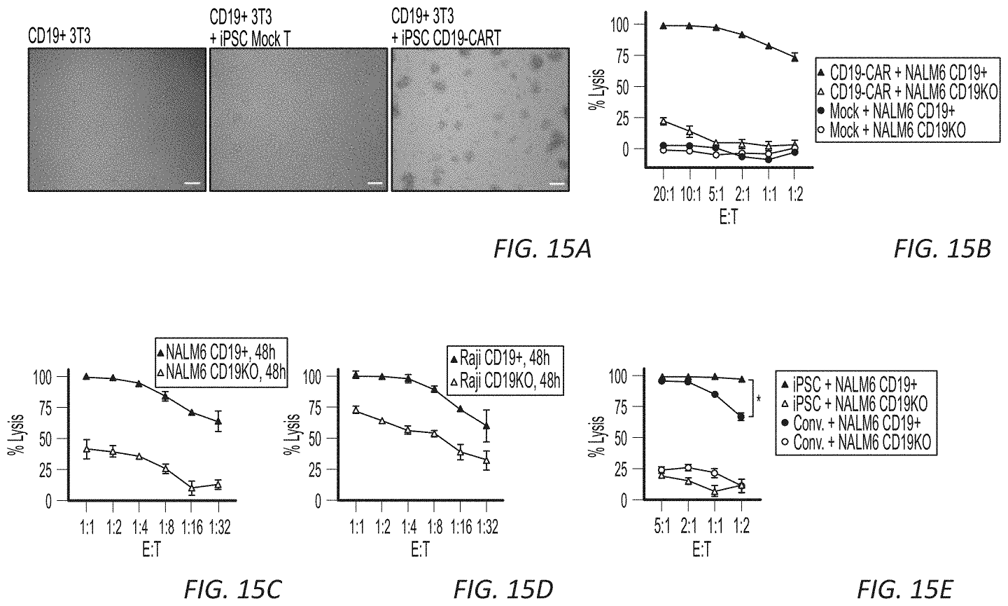

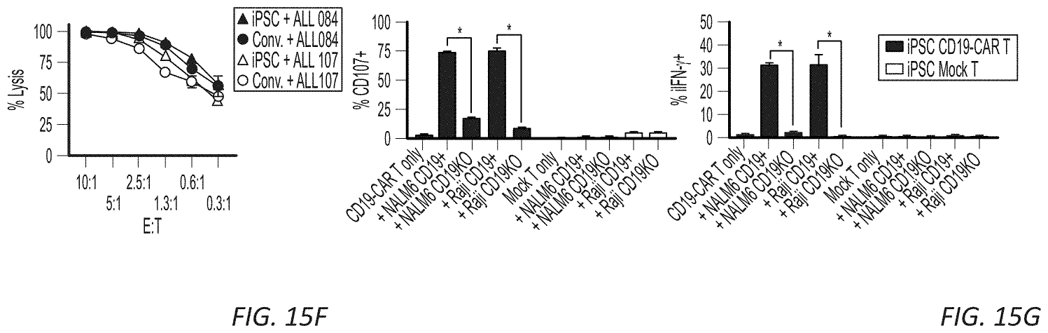

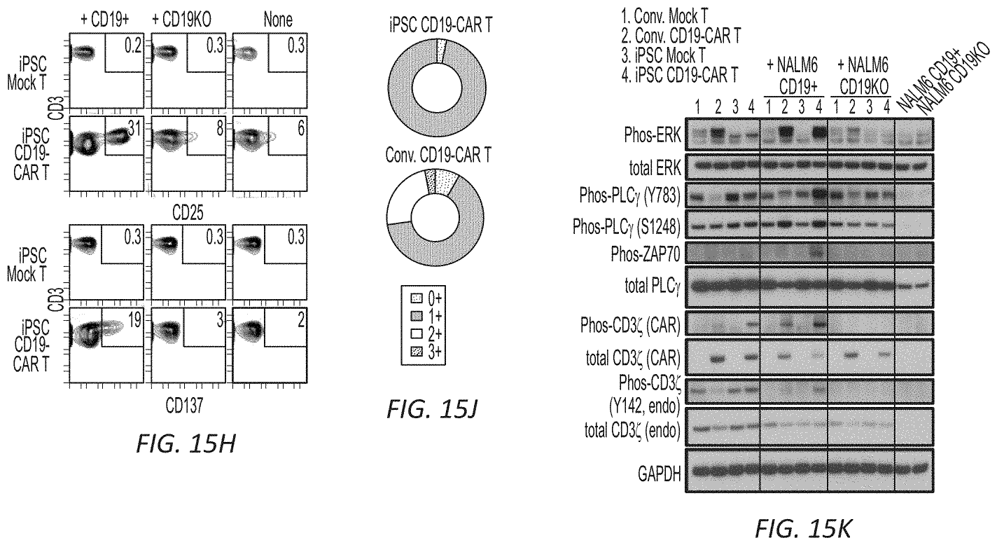

We next evaluated the effector function of REM expanded iPSC CD19-CAR T cells to lyse CD19 expressing targets in vitro. iPSC CAR T cells mediated potent CAR-directed cytolytic activity against CD19+ 3T3 cells (FIG. 15A), NALM6 cells (FIG. 15B-15C), and Raji cells (FIG. 15D), but not their CD19-negative counterparts. We use PBMC derived CD19-CAR T cells as comparison control, which was produced by clinically relevant procedure and did not go through REM expansion. Importantly the killing activity of iPSC CD19-CAR T cells was comparable or superior to conventional PBMC-derived CD19-CAR T cells from the same donor, as evidenced by iPSC CD19-CAR T cells exhibiting more potent lytic activity against CD19+ NALM6 cells at low E:T ratios (FIG. 15E), and showing comparable cytotoxicity against primary patient-derived CD19+ B-ALL cell (FIG. 15F). Upon CD19+ tumor cell stimulation, iPSC CD19-CAR T cells also demonstrated potent degranulation, expression of intracellular IFNγ, surface expression of activation markers CD25 and CD137/4-1BB, and Th1 cytokine release in an antigen-dependent manner (FIG. 15G-I). Interestingly, without antigenic stimulation, the levels of GM-CSF and IFN-γ are much lower in the supernatant from iPSC CD19-CAR T cells than that of conventional CD19-CAR T cells (FIG. 15I), which is consistent with lower basal ERK protein phosphorylation level (FIG. 15K) and suggests lower levels of CAR tonic signaling. Furthermore, upon serial challenge with CD19+ tumor cells, iPSC CD19-CAR T cells displayed decreased expression of PD-1, TIM-3 and LAG-3 as compared to conventional CD19-CAR T cells, indicating a less exhausted phenotype. (FIG. 15J).

Next, we explored CAR T cell signaling upon co-culture with either parental CD19⁺ or CD19 KO NALM6 cells. iPSC CD19-CAR T cells demonstrated ERK1/2 Thr202/Thr204, and PLCγ Ser1248 phosphorylation in an antigen specific manner that was comparable to that of conventional CD19-CAR T cells (FIG. 15K). However, the PLCγ Y783, ZAP70 and endogenous CD3ζ phosphorylation levels were higher in antigen stimulated iPSC CD19-CAR T cells than antigen stimulated conventional CD19-CAR T cells, which support the potent cytotoxicity activity. Interestingly, both the endogenous CD3ζ Y142 and CAR-associated CD3ζ phosphorylation in CD19-CAR T cells was suppressed by co-culture with CD19 negative NALM6 cells, indicating an immunosuppressive effect of cancer cells (FIG. 15K). Western blot analysis also confirmed that the iPSC CD19-CAR T cells expressed much lower levels of CAR transgene than conventional CAR T cells (FIG. 15K), which is consistent to flow cytometry data (FIG. 15H). It may also explain their lower activation level in the absence of antigen as measured by ERK phosphorylation (FIG. 15K) and cytokine secretion (FIG. 15I), since lower CAR expression has been show to favor lower tonic signaling (Eyquem et al., 2017).

The disclosed iPSC CD19-CAR T cells yield products with comparable or superior in vitro effector activity as compared to conventional CAR T cell expanded using clinically relevant methodologies.

Anti-Tumor Efficacy of iPSC-Derived CAR T Cells

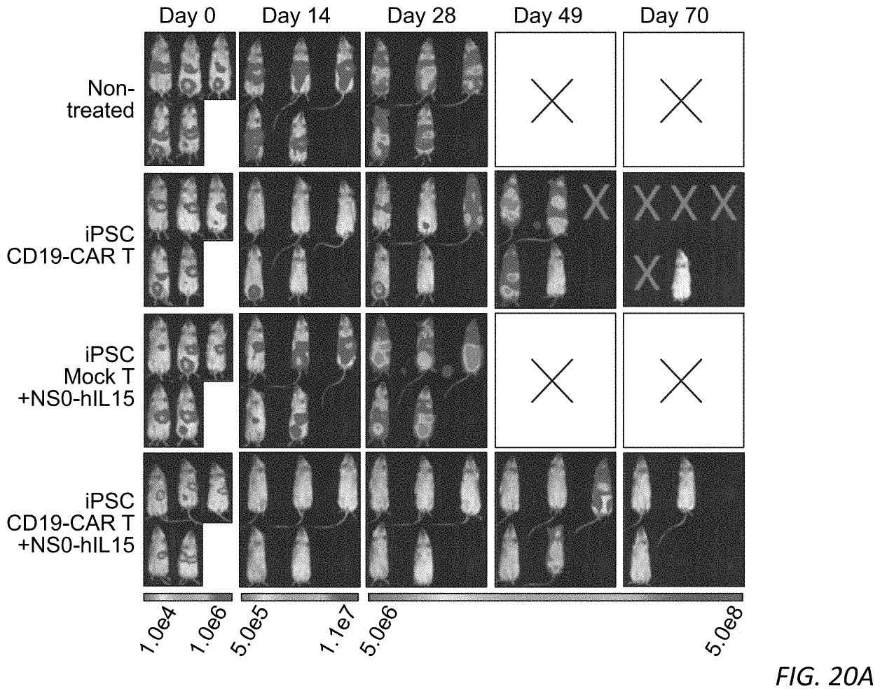

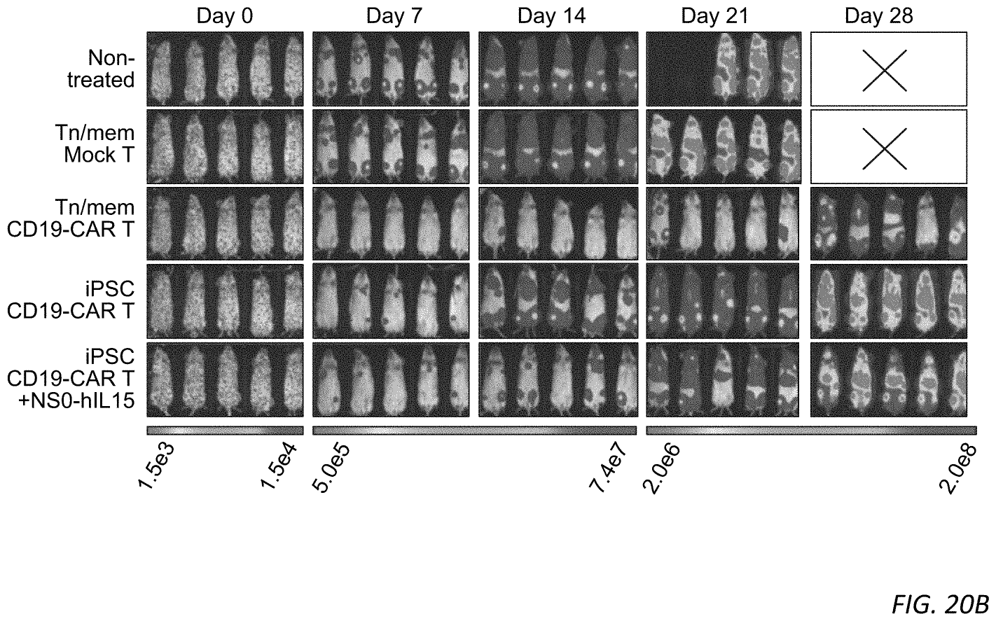

While the reduced CAR expression resulted in less activation in the absence of antigen (FIG. 14I and FIG. 15E), and might account for the less exhausted phenotype in the presence of antigen challenge (FIG. 15J), the iPSC CD19-CAR T cells still appeared to exhibit robust antigen-specific cytotoxic activity in vitro (FIG. 14A-F). However, to better evaluate the anti-tumor activity of these T cells, we next carried out in vivo therapeutic assays in mouse xenograft models using NALM6 cells expressing firefly luciferase to allow for bioluminescent imaging (images provided in FIGS. 20A-20B). In the intraperitoneal (i.p.) tumor model, i.p. administration of iPSC CD19-CAR T cells dramatically delayed tumor progression (FIG. 16B) and significantly prolonged the mouse survival (P=0.004) (FIG. 16C). Combination of iPSC CD19-CAR T cells with human IL15 secreting nurse cells (NS0-hIL15) further enhanced this therapeutic effect, leading to complete cure in 3 out 5 mice (FIG. 20A). The therapeutic benefit of iPSC CD19-CAR T cells was also demonstrated in a more aggressive intravenous (i.v.) mouse tumor model (FIG. 16E-16F), again showing significantly improved mouse survival (P=0.0035). In summary, the iPSC CD19-CAR T cells which were produced by PSC-ATO culture system from CAR expressing Tn/mem cells, demonstrated potent anti-tumor efficacy in vivo.

Discussion

Generation of T cells and CAR T cells using extrathymic culture systems, whether they are single-layer or 3D-organoid co-cultures, is challenge (Maeda et al., 2016; Montel-Hagen et al., 2019; Vizcardo et al., 2018; Vizcardo et al., 2013; Zhao et al., 2007). The first reported iPSC CAR T cells generated by a mono-layer co-culture system displayed an innate-like phenotype (i.e., CD8αα⁺), as well as less-efficient antigen-specific cytotoxicity and cytokine secretion compared to conventional CAR T cells (Themeli et al., 2013). We modified and optimized a 3D-organoid culture system that facilitated generation of mature and functional CD3+CD8αβ+ and CD3+CD4+ conventional T cells and TCR-transgenic T cells (Montel-Hagen et al., 2019), and we demonstrated for the first time the successful generation of iPSC CAR T cells with a conventional T cell phenotype and CAR T cell function. Specifically, by using Tn/mem-derived iPSCs that were gene modified to express the CAR, and an PSC-ATO culture system to drive differentiation, iPSC CAR T cells were generated expressing conventional CD5+CD7+TCRαβ+TCRγδ-CD8αβ+ T cell phenotypes, exhibited potent cytotoxic killing, and Th1 cytokine secretion activity that was comparable to conventional CAR T cells derived from the same donor. Such improvements validate the potential utility of iPSCs for generating therapeutic CAR T cell products.

It is also advantageous that our Tn/mem-derived iPSC CAR T cells displayed a more homogenous, monoclonal TCR repertoire, which was different from the polyclonal phenotype in ESC-derived T cells (Montel-Hagen et al., 2019; Nishimura et al., 2013). Even the use of terminal differentiated effector T cells to generate the iPSCs resulted in regenerated CD8αβ T cells that lost their antigen specificity by additional TCR rearrangement, with TCR stability only being induced upon TCR transduction of the iPSCs (Minagawa et al., 2018). This shows that starting with a less-differentiated Tn/mem population may have unique effects on TCR rearrangement during re-differentiation, which may or may not relate to the allelic exclusion effect of pre-existing TCR loci (Brady et al., 2010). Regardless, selection of Tn/mem-derived iPSC clones of a known and/or innocuous TCR to minimize potential graft-versus-host toxicities are relevant to the manufacture of an ‘off-the-shelf’ iPSC CAR T cell products. All disclosed iPSC CAR T cells and iPSC CAR NK cell described herein can be used as such.

The lower expression levels of MHC and dominance of CD8 on our iPSC CAR T cells may also relate to the unique effects of starting with Tn/mem-derived iPSC clones, or it may be related to the lack of thymic epithelial cells in the culture system (Vizcardo et al., 2018). While low MHC expression may be desirable for reducing T cell mediated rejection and facilitating iPSC CAR T cell persistence after adoptive transfer, it might be important to improve the balance between the CD4+ and CD8+ populations, since CD4+ CAR T cells have recently been shown to play important role in adoptive immune cell therapy (Wang et al., 2018). A more balanced CD4/CD8 lineage differentiation may be obtained by manipulating either the culture conditions during differentiation or the lineage selection pathways by gene editing (Singer et al., 2008).

We demonstrated for the first time that decreased CAR expression in the iPSC CAR T cells was related to the hyper-methylation of the EF1a promoter. While promoter methylation has been known to regulate gene expression (Hofmann et al., 2006), the differentiation-induced hyper-methylation of the CAR transgene promoter represents a new mechanism by which one might regulate CAR expression. Lowering CAR expression might be more desirable than previously appreciated, as it has been reported that optimal basal low expression of CAR can reduce tonic signaling and sustain CAR T cell functions (Eyquem et al., 2017). In our study, the basal pERK phosphorylation level and the basal GM-CSF/IFNγ secretion levels of iPSC CD19-CAR T cells without antigen stimulation was much lower than that seen in conventional CD19-CAR T cells. In contrast, upon antigen encounter, cell signaling by iPSC CD19-CAR T cells, as measured by pERK, PLCγ(Y782) and ZAP70 phosphorylation, as well as Th1 cytokine secretion was comparable to or higher than that of conventional CD19-CAR T cells, in spite of the lower CAR expression. Interestingly, while phosphorylation of both the endogenous and CAR-associated CD3ζ sequences was suppressed by NALM6 tumor cells (FIG. 15K), a phenomenon which has not been reported with anti-CD3 or antigen coated beads (Salter et al., 2018; Sun et al., 2020), the cytotoxic activity observed against CD19+αNALM6 tumors (FIG. 15E) suggest that iPSC CD19-CAR T cells overcame this suppression better than conventional CD19-CAR T cells. Together these data suggest iPSC CAR T cells may exhibit an antigen-specificity profile that is beneficial for both safety and efficacy.

Akbar, A. N., and Henson, S. M. (2011). Are senescence and exhaustion intertwined or unrelated processes that compromise immunity? Nature reviews Immunology 11, 289-295.

### REFERENCES FOR EXAMPLE 4

Alizadeh, D., Wong, R. A., Yang, X., Wang, D., Pecoraro, J. R., Kuo, C. F., Aguilar, B., Qi, Y., Ann, D. K., Starr, R., et al. (2019). IL15 Enhances CAR-T Cell Antitumor Activity by Reducing mTORC1 Activity and Preserving Their Stem Cell Memory Phenotype. Cancer Immunol Res 7, 759-772.

Brady, B. L., Steinel, N. C., and Bassing, C. H. (2010). Antigen receptor allelic exclusion: an update and reappraisal. Journal of immunology 185, 3801-3808.

Brown, C. E., Alizadeh, D., Starr, R., Weng, L., Wagner, J. R., Naranjo, A., Ostberg, J. R., Blanchard, M. S., Kilpatrick, J., Simpson, J., et al. (2016). Regression of Glioblastoma after Chimeric Antigen Receptor T-Cell Therapy. The New England journal of medicine 375, 2561-2569.

Deuse, T., Hu, X., Gravina, A., Wang, D., Tediashvili, G., De, C., Thayer, W. O., Wahl, A., Garcia, J. V., Reichenspurner, H., et al. (2019). Hypoimmunogenic derivatives of induced pluripotent stem cells evade immune rejection in fully immunocompetent allogeneic recipients. Nature biotechnology 37, 252-258.

Eyquem, J., Mansilla-Soto, J., Giavridis, T., van der Stegen, S. J., Hamieh, M., Cunanan, K. M., Odak, A., Gonen, M., and Sadelain, M. (2017). Targeting a CAR to the TRAC locus with CRISPR/Cas9 enhances tumour rejection. Nature 543, 113-117.

Hofmann, A., Kessler, B., Ewerling, S., Kabermann, A., Brem, G., Wolf, E., and Pfeifer, A. (2006). Epigenetic regulation of lentiviral transgene vectors in a large animal model. Mol Ther 13, 59-66.

Inoue, H., Nagata, N., Kurokawa, H., and Yamanaka, S. (2014). iPS cells: a game changer for future medicine. The EMBO journal 33, 409-417.

June, C. H., O'Connor, R. S., Kawalekar, O. U., Ghassemi, S., and Milone, M. C. (2018). CAR T cell immunotherapy for human cancer. Science 359, 1361-1365.

June, C. H., and Sadelain, M. (2018). Chimeric Antigen Receptor Therapy. The New England journal of medicine 379, 64-73.

Kennedy, M., Awong, G., Sturgeon, C. M., Ditadi, A., LaMotte-Mohs, R., Zuniga-Pflucker, J. C., and Keller, G. (2012). T lymphocyte potential marks the emergence of definitive hematopoietic progenitors in human pluripotent stem cell differentiation cultures. Cell reports 2, 1722-1735.

Kohl, U., Arsenieva, S., Holzinger, A., and Abken, H. (2018). CAR T Cells in Trials: Recent Achievements and Challenges that Remain in the Production of Modified T Cells for Clinical Applications. Hum Gene Ther 29, 559-568.

Levine, B. L., Miskin, J., Wonnacott, K., and Keir, C. (2017). Global Manufacturing of CAR T Cell Therapy. Molecular therapy Methods & clinical development 4, 92-101.

Lin, J. K., Muffly, L. S., Spinner, M. A., Barnes, J. I., Owens, D. K., and Goldhaber-Fiebert, J. D. (2019). Cost Effectiveness of Chimeric Antigen Receptor T-Cell Therapy in Multiply Relapsed or Refractory Adult Large B-Cell Lymphoma. J Clin Oncol 37, 2105-2119.

Long, A. H., Haso, W. M., Shern, J. F., Wanhainen, K. M., Murgai, M., Ingaramo, M., Smith, J. P., Walker, A. J., Kohler, M. E., Venkateshwara, V. R., et al. (2015). 4-1BB costimulation ameliorates T cell exhaustion induced by tonic signaling of chimeric antigen receptors. Nature medicine 21, 581-590.

Maeda, T., Nagano, S., Ichise, H., Kataoka, K., Yamada, D., Ogawa, S., Koseki, H., Kitawaki, T., Kadowaki, N., Takaori-Kondo, A., et al. (2016). Regeneration of CD8alphabeta T Cells from T-cell-Derived iPSC Imparts Potent Tumor Antigen-Specific Cytotoxicity. Cancer research 76, 6839-6850.

McLellan, A. D., and Ali Hosseini Rad, S. M. (2019). Chimeric antigen receptor T cell persistence and memory cell formation. Immunology and cell biology 97, 664-674.

Minagawa, A., Yoshikawa, T., Yasukawa, M., Hotta, A., Kunitomo, M., Iriguchi, S., Takiguchi, M., Kassai, Y., Imai, E., Yasui, Y., et al. (2018). Enhancing T Cell Receptor Stability in Rejuvenated iPSC-Derived T Cells Improves Their Use in Cancer Immunotherapy. Cell stem cell 23, 850-858.e854.

Montel-Hagen, A., Seet, C. S., Li, S., Chick, B., Zhu, Y., Chang, P., Tsai, S., Sun, V., Lopez, S., Chen, H. C., et al. (2019). Organoid-Induced Differentiation of Conventional T Cells from Human Pluripotent Stem Cells. Cell stem cell 24, 376-389.e378.

Morgan, M. A., and Schambach, A. (2018). Engineering CAR-T Cells for Improved Function Against Solid Tumors. Frontiers in immunology 9, 2493.

Murry, C. E., and Keller, G. (2008). Differentiation of embryonic stem cells to clinically relevant populations: lessons from embryonic development. Cell 132, 661-680.

Nishimura, T., Kaneko, S., Kawana-Tachikawa, A., Tajima, Y., Goto, H., Zhu, D., Nakayama-Hosoya, K., Iriguchi, S., Uemura, Y., Shimizu, T., et al. (2013). Generation of rejuvenated antigen-specific T cells by reprogramming to pluripotency and redifferentiation. Cell stem cell 12, 114-126.

Okita, K., Yamakawa, T., Matsumura, Y., Sato, Y., Amano, N., Watanabe, A., Goshima, N., and Yamanaka, S. (2013). An efficient nonviral method to generate integration-free human-induced pluripotent stem cells from cord blood and peripheral blood cells. Stem cells 31, 458-466.

Palazon, A., Tyrakis, P. A., Macias, D., Velica, P., Rundqvist, H., Fitzpatrick, S., Vojnovic, N., Phan, A. T., Loman, N., Hedenfalk, I., et al. (2017). An HIF-1alpha/VEGF-A Axis in Cytotoxic T Cells Regulates Tumor Progression. Cancer cell 32, 669-683 e665.

Pavlacky, J., and Polak, J. (2020). Technical Feasibility and Physiological Relevance of Hypoxic Cell Culture Models. Front Endocrinol (Lausanne) 11, 57.

Poirot, L., Philip, B., Schiffer-Mannioui, C., Le Clerre, D., Chion-Sotinel, I., Derniame, S., Potrel, P., Bas, C., Lemaire, L., Galetto, R., et al. (2015). Multiplex Genome-Edited T-cell Manufacturing Platform for “Off-the-Shelf” Adoptive T-cell Immunotherapies. Cancer research 75, 3853-3864.

Popplewell, L., X., W., S., B., J., W., Naranjo, A., A., A., J., P., L., L., W., C., E., B., et al. (2018). CD19-CAR Therapy Using Naive/Memory or Central Memory T Cells Integrated into the Autologous Stem Cell Transplant Regimen for Patients with B-NHL. Blood 132, 610.

Porter, D. L., Levine, B. L., Kalos, M., Bagg, A., and June, C. H. (2011). Chimeric antigen receptor-modified T cells in chronic lymphoid leukemia. The New England journal of medicine 365, 725-733.

Programs, I.-o. (2019). Programmed Cellular Immunotherapies Overview of Immuno-Oncology Programs Forward-Looking Statements.

Salter, A. I., Ivey, R. G., Kennedy, J. J., Voillet, V., Rajan, A., Alderman, E. J., Voytovich, U. J., Lin, C., Sommermeyer, D., Liu, L., et al. (2018). Phosphoproteomic analysis of chimeric antigen receptor signaling reveals kinetic and quantitative differences that affect cell function. Science signaling 11.

Samer K. Khaled, M., Suzette Blanchard, PhD, Xiuli Wang, PhD, Jamie Wagner, BS, Araceli Naranjo, BS, Jennifer Simpson, Sandra Thomas, PhD, Julie Ostberg, PhD, Christine Brown, PhD, Stephen J Forman (2018). Adult Patients with ALL Treated with CD62L+ T Naïve/Memory-Enriched T Cells Expressing a CD19-CAR Mediate Potent Antitumor Activity with a Low Toxicity Profile. Blood 132

Singer, A., Adoro, S., and Park, J. H. (2008). Lineage fate and intense debate: myths, models and mechanisms of CD4- versus CD8-lineage choice. Nature reviews Immunology 8, 788-801.

Sun, C., Shou, P., Du, H., Hirabayashi, K., Chen, Y., Herring, L. E., Ahn, S., Xu, Y., Suzuki, K., Li, G., et al. (2020). THEMIS-SHP1 Recruitment by 4-1BB Tunes LCK-Mediated Priming of Chimeric Antigen Receptor-Redirected T Cells. Cancer cell.

Themeli, M., Kloss, C. C., Ciriello, G., Fedorov, V. D., Perna, F., Gonen, M., and Sadelain, M. (2013). Generation of tumor-targeted human T lymphocytes from induced pluripotent stem cells for cancer therapy. Nature biotechnology 31, 928-933.

Themeli, M., Riviere, I., and Sadelain, M. (2015). New cell sources for T cell engineering and adoptive immunotherapy. Cell stem cell 16, 357-366.

Thommen, D. S., and Schumacher, T. N. (2018). T Cell Dysfunction in Cancer. Cancer cell 33, 547-562.

Timmermans, F., Velghe, I., Vanwalleghem, L., De Smedt, M., Van Coppernolle, S., Taghon, T., Moore, H. D., Leclercq, G., Langerak, A. W., Kerre, T., et al. (2009). Generation of T cells from human embryonic stem cell-derived hematopoietic zones. Journal of immunology 182, 6879-6888.

Vizcardo, R., Klemen, N. D., Islam, S. M. R., Gurusamy, D., Tamaoki, N., Yamada, D., Koseki, H., Kidder, B. L., Yu, Z., Jia, L., et al. (2018). Generation of Tumor Antigen-Specific iPSC-Derived Thymic Emigrants Using a 3D Thymic Culture System. Cell reports 22, 3175-3190.

Vizcardo, R., Masuda, K., Yamada, D., Ikawa, T., Shimizu, K., Fujii, S., Koseki, H., and Kawamoto, H. (2013). Regeneration of human tumor antigen-specific T cells from iPSCs derived from mature CD8(+) T cells. Cell stem cell 12, 31-36.

Wang, D., Aguilar, B., Starr, R., Alizadeh, D., Brito, A., Sarkissian, A., Ostberg, J. R., Forman, S. J., and Brown, C. E. (2018). Glioblastoma-targeted CD4+ CAR T cells mediate superior antitumor activity. JCI insight 3.

Wang, R., Dillon, C. P., Shi, L. Z., Milasta, S., Carter, R., Finkelstein, D., McCormick, L. L., Fitzgerald, P., Chi, H., Munger, J., et al. (2011a). The transcription factor Myc controls metabolic reprogramming upon T lymphocyte activation. Immunity 35, 871-882.

Wang, X., Berger, C., Wong, C. W., Forman, S. J., Riddell, S. R., and Jensen, M. C. (2011b). Engraftment of human central memory-derived effector CD8+T cells in immunodeficient mice. Blood 117, 1888-1898.

Yang, Y., Jacoby, E., and Fry, T. J. (2015). Challenges and opportunities of allogeneic donor-derived CAR T cells. Current opinion in hematology 22, 509-515.

Zah, E., Nam, E., Bhuvan, V., Tran, U., Ji, B. Y., Gosliner, S. B., Wang, X., Brown, C. E., and Chen, Y. Y. (2020). Systematically optimized BCMA/CS1 bispecific CAR-T cells robustly control heterogeneous multiple myeloma. Nat Commun 11, 2283.

Zhao, Y., Parkhurst, M. R., Zheng, Z., Cohen, C. J., Riley, J. P., Gattinoni, L., Restifo, N. P., Rosenberg, S. A., and Morgan, R. A. (2007). Extrathymic generation of tumor-specific T cells from genetically engineered human hematopoietic stem cells via Notch signaling. Cancer research 67, 2425-2429.

### REFERENCES FOR METHODS IN EXAMPLE 4

1. Samer K. Khaled, M., Suzette Blanchard, PhD, Xiuli Wang, PhD, Jamie Wagner, BS, Araceli Naranjo, BS, Jennifer Simpson, Sandra Thomas, PhD, Julie Ostberg, PhD, Christine Brown, PhD, Stephen J Forman, Adult Patients with ALL Treated with CD62L+T Naïve/Memory-Enriched T Cells Expressing a CD19-CAR Mediate Potent Antitumor Activity with a Low Toxicity Profile. Blood, 2018. 132 (1).

2. Popplewell, L., et al., CD19-CAR Therapy Using Naive/Memory or Central Memory T Cells Integrated into the Autologous Stem Cell Transplant Regimen for Patients with B-NHL. Blood, 2018. 132: p. 610.

3. Nicholson, I. C., et al., Construction and characterisation of a functional CD19 specific single chain Fv fragment for immunotherapy of B lineage leukaemia and lymphoma. Mol Immunol, 1997. 34(16-17): p. 1157-65.

4. Donnelly, M. L. L., et al., Analysis of the aphthovirus 2A/2B polyprotein ‘cleavage’ mechanism indicates not a proteolytic reaction, but a novel translational effect: a putative ribosomal ‘skip’. J Gen Virol, 2001. 82(Pt 5): p. 1013-1025.

5. Jonnalagadda, M., et al., Chimeric antigen receptors with mutated IgG4 Fc spacer avoid fc receptor binding and improve T cell persistence and antitumor efficacy. Mol Ther, 2015. 23(4): p. 757-68.

6. Urak, R., et al., Ex vivo Akt inhibition promotes the generation of potent CD19CAR T cells for adoptive immunotherapy. J Immunother Cancer, 2017. 5: p. 26.

7. Wang, X., et al., A transgene-encoded cell surface polypeptide for selection, in vivo tracking, and ablation of engineered cells. Blood, 2011. 118(5): p. 1255-63.

8. Okita, K., et al., An efficient nonviral method to generate integration-free human-induced pluripotent stem cells from cord blood and peripheral blood cells. Stem Cells, 2013. 31(3): p. 458-66.

9. Wang, X., et al., Phenotypic and functional attributes of lentivirus-modified CD19-specific human CD8+ central memory T cells manufactured at clinical scale. J Immunother, 2012. 35(9): p. 689-701.

10. Black, J. and J. M. Vos, Establishment of an oriP/EBNA1-based episomal vector transcribing human genomic beta-globin in cultured murine fibroblasts. Gene Ther, 2002. 9(21): p. 1447-54.

11. Chin, C. J., et al., Genetic Tagging During Human Mesoderm Differentiation Reveals Tripotent Lateral Plate Mesodermal Progenitors. Stem Cells, 2016. 34(5): p. 1239-50.

12. Evseenko, D., et al., Mapping the first stages of mesoderm commitment during differentiation of human embryonic stem cells. Proc Natl Acad Sci USA, 2010. 107(31): p. 13742-7.

13. Montel-Hagen, A., et al., Organoid-Induced Differentiation of Conventional T Cells from Human Pluripotent Stem Cells. Cell Stem Cell, 2019. 24(3): p. 376-389 e8.

14. Wang, X., et al., Engraftment of human central memory-derived effector CD8+ T cells in immunodeficient mice. Blood, 2011. 117(6): p. 1888-98.

15. Wang, X., et al., CMVpp65 Vaccine Enhances the Antitumor Efficacy of Adoptively Transferred CD19-Redirected CMV-Specific T Cells. Clin Cancer Res, 2015. 21(13): p. 2993-3002.

16. van Dongen, J. J., et al., Design and standardization of PCR primers and protocols for detection of clonal immunoglobulin and T-cell receptor gene recombinations in suspect lymphoproliferation: report of the BIOMED-2 Concerted Action BMH4-CT98-3936. Leukemia, 2003. 17(12): p. 2257-317.

17. Langerak, A. W., et al., EuroClonality/BIOMED-2 guidelines for interpretation and reporting of Ig/TCR clonality testing in suspected lymphoproliferation. Leukemia, 2012. 26(10): p. 2159-71.

18. Brown, C. E., et al., Biophotonic cytotoxicity assay for high-throughput screening of cytolytic killing. J Immunol Methods, 2005. 297(1-2): p. 39-52.

19. Wang, D., et al., In Vitro Tumor Cell Rechallenge For Predictive Evaluation of Chimeric Antigen Receptor T Cell Antitumor Function. J Vis Exp, 2019(144).

20. Robinson, M. D., D. J. McCarthy, and G. K. Smyth, edgeR: a Bioconductor package for differential expression analysis of digital gene expression data. Bioinformatics, 2010. 26(1): p. 139-40.

21. Subramanian, A., et al., Gene set enrichment analysis: a knowledge-based approach for interpreting genome-wide expression profiles. Proc Natl Acad Sci USA, 2005. 102(43): p. 15545-50.

22. Mootha, V. K., et al., PGC-1alpha-responsive genes involved in oxidative phosphorylation are coordinately downregulated in human diabetes. Nat Genet, 2003. 34(3): p. 267-73.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for preparing a composition of T cells or NK cells expressing a chimeric antigen receptor (CAR), the method comprising:
(a) isolating a population of peripheral blood mononuclear cells (PBMCs), naïve T (Tₐ) cells, memory T (Tₘₑₘ) cells, naïve and memory T cells (T_{n/mem}), or a combination thereof;
(b) generating induced pluripotent stem cells (iPSCs) from the PBMCs, Tₙcells, Tₘₑₘ cells, or T_{n/mem} cells, or combination thereof;
(c) contacting the iPSCs with a vector encoding the CAR, thereby creating CAR iPSCs; and
(d) differentiating the CAR iPSCs into CAR T cells or CAR NK cells.

2. The method of claim 1, wherein the PBMCs, Tₙ cells, Tₘₑₘ cells, or T_{n/mem} cells, or combination thereof are human or are isolated from human blood.

3. The method of claim 1, wherein the PBMC cells are CD14⁻, CD25⁻, and CD26L⁺.

4. The method of claim 1, wherein the iPSCs are generated by contacting the PBMCs, Tₙ cells, Tₙ cells, or T_{n/mem} cells, or combination thereof with one or more of OCT3/4, OCT3, OCT4, SOX2, KLF4, L-MYC, C-MYC, LIN28, or short hairpin RNA targeting TP53 (shRNA-TP53).

5. The method of claim 1, wherein the iPSCs are genetically modified.

6. The method of claim 5, wherein the genetic modification comprises knock out of one or more genes, wherein the one or more genes comprise one or more of TRAC, TRBC, B2M, CIITA, or combinations thereof.

7. The method of claim 5, wherein genetic modification methods comprise gene editing, homologous recombination, nonhomologous recombination, RNA-mediated genetic modification, DNA-mediated genetic modification, zinc finger nucleases, meganucleases, TALEN, or CRISPR/CAS9.

8. The method of claim 1, wherein the step of differentiating the CAR iPSCs into CAR T cells or CAR NK cells comprises differentiating the CAR-expressing iPSCs into embryonic mesodermal progenitor (EMP) cells and differentiating the EMP into CAR T cells.

9. The method of claim 8, wherein the EMP cells are CD56⁺ and CD326⁻.

10. The method of claim 1, wherein the step of differentiating the CAR iPSCs into CAR T cells or CAR NK cells comprises differentiating the CAR-expressing iPSCs into embryonic mesodermal progenitor (EMP) cells and differentiating the EMP into CAR NK cells.

11. The method of claim 10, wherein EMP cells are CD56⁺ and CD326⁻.

12. The method of claim 1, wherein the step of differentiating the CAR iPSCs into CAR T cells or CAR NK cells comprises differentiating the CAR iPSCs into CD34⁺ hematopoietic stem and progenitor cells (HSPCs) and differentiating the HSPCs into CAR T cells.

13. The method of claim 1, wherein the step of differentiating the CAR iPSCs into CAR T cells or CAR NK cells comprises differentiating the CAR iPSCs into CD34+ HSPCs and differentiating the HSPCs into CAR NK cells.

14. The method of claim 1, wherein the step of differentiating the CAR iPSCs into CAR T cells comprises using a nanofiber matrix-based culture system.

15. The method of claim 1, wherein the step of differentiating the CAR iPSCs into CAR NK cells comprises using a nanofiber matrix-based culture system.

16. The method of claim 1, wherein the CAR is specific for a tumor and/or toxin.

17. The method of claim 1, wherein the CAR targets any one or more of carbonic anhydrase IX (CAIX), carcinoembryonic antigen (CEA), CDS, CD6, CD7, CD10, CD19, CD20, CD22, CD30, CD33, CD34, CD38, CD41, CD44, CD49f, CD56, CD74, CD123, CD133, CD138, CS1, chlorotoxin receptor, an antigen of a cytomegalovirus (CMV) infected cell (e.g., a cell surface antigen), epithelial glycoprotein (EGP 2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptors, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), light chain kinase insert domain receptor (KDR), Lewis A (CA19.9), Lewis Y (LeY), LI cell adhesion molecule (LICAM), melanoma antigen family A, 1 (MAGE-AI), Mucin 16 (Muc-16), Mucin 1 (Muc-1), Mesothelin (MSLN), NKG2D ligands, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor R2 (VEGF-R2), Wilms tumor protein (WT-1), or combinations thereof.

18. The method of claim 1, wherein the CAR is bispecific.

19. The method of claim 1, wherein the chimeric antigen receptor comprises: at least one targeting domain, a spacer, a transmembrane domain, a co-stimulatory domain, and a CD3 signaling domain.

20. A composition comprising the iPSC-derived CAR T cells or CAR NK cells of claim 1.

21. The composition of claim 20, wherein the CAR T cells comprise one or more of helper T cells, cytotoxic T cells, memory T cells, naïve T cells, regulatory T cells, natural killer T cells, or combinations thereof.

22. The composition of claim 20, wherein the CAR T cells comprise CD3⁺, CDS⁺, CD7⁺, and TCRαβ⁺.

23. A method of increasing survival of a subject having cancer comprising administering the composition of claim 20 to the patient.

24. A method of treating a cancer in a patient comprising administering the composition of claim 20 to the patient.

25. A method of reducing or ameliorating a symptom associated with a cancer in a patient comprising administering the composition of claim 20 to the patient.

26. The method of claim 23, wherein the composition is administered locally or systemically.

27. The method of claim 23, wherein the composition is administered by single or repeat dosing.
